SEQUENCE LISTING (29 sequences)

SEQ ID NO: 1 - DNA, 9056 bp, Homo sapiens
  source 1..9056: mol_type = genomic DNA
ttaattaactgcagacgccagctgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttctagaggcgcgccaagcttggtacccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccacccctaggacccaagtcgcagccgttggatcaggtaagtatcaaggttacaagacaggtttaaggagaccaatagaaactgggcttgtcgagacagagaagactcttgcgtttctgataggcacctattggtcttactgacatccactttgcctttctctccacaggtgatgatatcgcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatcttatcatgtccatcgataaggatctaggaacccctagtgatgggcgcgccatcggatcccgggcccgtcgacgcgccggcgtctagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacagctgcattaatgaatcctgcaggcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctctatgagcggatacatatttgaatgtatttagaaaaataaacaaataggggttccgcgtcatgagattatcaaaaaggatcttcacctagatccttttcacgtagaaagccagtccgcagaaacggtgctgaccccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttcacacaaaaaaccaacacacagatgtaatgaaaataaagatattttattgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgtccattcgccattcaggctgcgcaactgttgggaagggctgcag

SEQ ID NO: 2 - DNA, 8818 bp, Homo sapiens
  source 1..8818: mol_type = genomic DNA
ttaattaactgcagacgccagctgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttctagaggcgcgccaagcttggtaccctagggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtgcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatcttatcatgtccatcgataaggatctaggaacccctagtgatgggcgcgccatcggatcccgggcccgtcgacgcgccggcgtctagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacagctgcattaatgaatcctgcaggcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctctatgagcggatacatatttgaatgtatttagaaaaataaacaaataggggttccgcgtcatgagattatcaaaaaggatcttcacctagatccttttcacgtagaaagccagtccgcagaaacggtgctgaccccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttcacacaaaaaaccaacacacagatgtaatgaaaataaagatattttattgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgtccattcgccattcaggctgcgcaactgttgggaagggctgcag

SEQ ID NO: 3 - DNA, 9177 bp, Homo sapiens
  source 1..9177: mol_type = genomic DNA
ttaattaactgcagacgccagctgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttctagaggcgcgccaagcttggtaccgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgtcgacaagaggtaagggtttaagggatggttggttggtggggtattaatgtttaattacctgttttacaggcctgaaatcacttggttttaggttgggaagcttgatatccctgcagggcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatcttatcatgtccatcgataaggatctaggaacccctagtgatgggcgcgccatcggatcccgggcccgtcgacgcgccggcgtctagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacagctgcattaatgaatcctgcaggcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctctatgagcggatacatatttgaatgtatttagaaaaataaacaaataggggttccgcgtcatgagattatcaaaaaggatcttcacctagatccttttcacgtagaaagccagtccgcagaaacggtgctgaccccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttcacacaaaaaaccaacacacagatgtaatgaaaataaagatattttattgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgtccattcgccattcaggctgcgcaactgttgggaagggctgcag

SEQ ID NO: 4 - DNA, 8934 bp, Homo sapiens
  source 1..8934: mol_type = genomic DNA
ttaattaactgcagacgccagctgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttctagaggcgcgccaagcttggtaccaaaatcaacgggactttccaaaatgtcgtaataaccccgccccgttgacgcaaatgggcggtaggcgtgtacggtgggaggtctatataagcagagctcgtttagtgaaccgtcagaattctcgagtgatcgaaagagcctgctaaagcaaaaaagaagtcaccgtcgacaagaggtaagggtttaagggatggttggttggtggggtattaatgtttaattacctgttttacaggcctgaaatcacttggttttaggttgggaagcttgatatccctgcagggcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatcttatcatgtccatcgataaggatctaggaacccctagtgatgggcgcgccatcggatcccgggcccgtcgacgcgccggcgtctagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacagctgcattaatgaatcctgcaggcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctctatgagcggatacatatttgaatgtatttagaaaaataaacaaataggggttccgcgtcatgagattatcaaaaaggatcttcacctagatccttttcacgtagaaagccagtccgcagaaacggtgctgaccccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttcacacaaaaaaccaacacacagatgtaatgaaaataaagatattttattgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgtccattcgccattcaggctgcgcaactgttgggaagggctgcag

SEQ ID NO: 5 - DNA, 8837 bp, Homo sapiens
  source 1..8837: mol_type = genomic DNA
ttaattaactgcagacgccagctgttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttctagaggcgcgccaagcttggtaccttcgcatattaaggtgacgcgtgtggcctcgaacaccgagcgaccctgcagcgacccgcttaaaagagtcgacaagaggtaagggtttaagggatggttggttggtggggtattaatgtttaattacctgttttacaggcctgaaatcacttggttttaggttgggaagcttgatatccctgcagggcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatcttatcatgtccatcgataaggatctaggaacccctagtgatgggcgcgccatcggatcccgggcccgtcgacgcgccggcgtctagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacagctgcattaatgaatcctgcaggcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctctatgagcggatacatatttgaatgtatttagaaaaataaacaaataggggttccgcgtcatgagattatcaaaaaggatcttcacctagatccttttcacgtagaaagccagtccgcagaaacggtgctgaccccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttcacacaaaaaaccaacacacagatgtaatgaaaataaagatattttattgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgtccattcgccattcaggctgcgcaactgttgggaagggctgcag

SEQ ID NO: 6 - protein, 1411 aa, Homo sapiens
MASISEPVTFREFCPLYYLLNAIPTKIQKGFRSIVVYLTALDTNGDYIAVGSSIGMLYLYCRHLNQMRKYNFEGKTESITVVKLLSCFDDLVAAGTASGRVAVFQLVSSLPGRNKQLRRFDVTGIHKNSITALAWSPNGMKLFSGDDKGKIVYSSLDLDQGLCNSQLVLEEPSSIVQLDYSQKVLLVSTLQRSLLFYTEEKSVRQIGTQPRKSTGKFGACFIPGLCKQSDLTLYASRPGLRLWKADVHGTVQATFILKDAFAGGVKPFELHPRLESPNSGSCSLPERHLGLVSCFFQEGWVLSWNEYSIYLLDTVNQATVAGLEGSGDIVSVSCTENEIFFLKGDRNIIRISSRPEGLTSTVRDGLEMSGCSERVHVQQAEKLPGATVSETRLRGSSMASSVASEPRSRSSSLNSTDSGSGLLPPGLQATPELGKGSQPLSQRFNAISSEDFDQELVVKPIKVKRKKKKKKTEGGSRSTCHSSLESTPCSEFPGDSPQSLNTDLLSMTSSVLGSSVDQLSAESPDQESSFNGEVNGVPQENTDPETFNVLEVSGSMPDSLAEEDDIRTEMPHCHHAHGRELLNGAREDVGGSDVTGLGDEPCPADDGPNSTQLPFQEQDSSPGAHDGEDIQPIGPQSTFCEVPLLNSLTVPSSLSWAPSAEQWLPGTRADEGSPVEPSQEQDILTSMEASGHLSTNLWHAVTDDDTGQKEIPISERVLGSVGGQLTPVSALAASTHKPWLEQPPRDQTLTSSDEEDIYAHGLPSSSSETSVTELGPSCSQQDLSRLGAEDAGLLKPDQFAESWMGYSGPGYGILSLVVSEKYIWCLDYKGGLFCSALPGAGLRWQKFEDAVQQVAVSPSGALLWKIEQKSNRAFACGKVTIKGKRHWYEALPQAVFVALSDDTAWIIRTSGDLYLQTGLSVDRPCARAVKVDCPYPLSQITARNNVVWALTEQRALLYREGVSSFCPEGEQWKCDIVSERQALEPVCITLGDQQTLWALDIHGNLWFRTGIISKKPQGDDDHWWQVSITDYVVFDQCSLFQTIIHATHSVATAAQAPVEKVADKLRMAFWSQQLQCQPSLLGVNNSGVWISSGKNEFHVAKGSLIGTYWNHVVPRGTASATKWAFVLASAAPTKEGSFLWLCQSSKDLCSVSAQSAQSRPSTVQLPPEAEMRAYAACQDALWALDSLGQVFIRTLSKSCPTGMHWTRLDLSQLGAVKLTSLACGNQHIWACDSRGGVYFRVGTQPLNPSLMLPAWIMIEPPVQPAGVSLVSVHSSPNDQMLWVLDSRWNVHVRTGITEEMPVGTAWEHVPGLQACQLALSTRTVWARCPNGDLARRYGVTDKNPAGDYWKKIPGSVSCFTVTASDELWAVGPPGYLLQRLTKTFSHSHGTQKSSQAAMPHPEDLEDEWEVI

SEQ ID NO: 7 - DNA, 4778 bp, Homo sapiens
  source 1..4778: mol_type = genomic DNA
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccacccctaggacccaagtcgcagccgttggatcaggtaagtatcaaggttacaagacaggtttaaggagaccaatagaaactgggcttgtcgagacagagaagactcttgcgtttctgataggcacctattggtcttactgacatccactttgcctttctctccacaggtgatgatatcgcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatctta

SEQ ID NO: 8 - DNA, 225 bp, Homo sapiens
  source 1..225: mol_type = genomic DNA
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 9 - DNA, 4537 bp, Homo sapiens
  source 1..4537: mol_type = genomic DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtgcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatctta

SEQ ID NO: 10 - DNA, 164 bp, Homo sapiens
  source 1..164: mol_type = genomic DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 11 - DNA, 4899 bp, Homo sapiens
  source 1..4899: mol_type = genomic DNA
gctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgtcgacaagaggtaagggtttaagggatggttggttggtggggtattaatgtttaattacctgttttacaggcctgaaatcacttggttttaggttgggaagcttgatatccctgcagggcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatctta

SEQ ID NO: 12 - DNA, 407 bp, Homo sapiens
  source 1..407: mol_type = genomic DNA
gctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggc

SEQ ID NO: 13 - DNA, 4657 bp, Homo sapiens
  source 1..4657: mol_type = genomic DNA
caaaatcaacgggactttccaaaatgtcgtaataaccccgccccgttgacgcaaatgggcggtaggcgtgtacggtgggaggtctatataagcagagctcgtttagtgaaccgtcagaattctcgagtgatcgaaagagcctgctaaagcaaaaaagaagtcaccgtcgacaagaggtaagggtttaagggatggttggttggtggggtattaatgtttaattacctgttttacaggcctgaaatcacttggttttaggttgggaagcttgatatccctgcagggcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatctta

SEQ ID NO: 14 - DNA, 159 bp, Homo sapiens
  source 1..159: mol_type = genomic DNA
caaaatcaacgggactttccaaaatgtcgtaataaccccgccccgttgacgcaaatgggcggtaggcgtgtacggtgggaggtctatataagcagagctcgtttagtgaaccgtcagaattctcgagtgatcgaaagagcctgctaaagcaaaaaagaa

SEQ ID NO: 15 - DNA, 4559 bp, Homo sapiens
  source 1..4559: mol_type = genomic DNA
ttcgcatattaaggtgacgcgtgtggcctcgaacaccgagcgaccctgcagcgacccgcttaaaagagtcgacaagaggtaagggtttaagggatggttggttggtggggtattaatgtttaattacctgttttacaggcctgaaatcacttggttttaggttgggaagcttgatatccctgcagggcggccgccgcatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgaccttaacttgtttattgcagcttataatggttacaaataaagcaatagcatcacaaatttcacaaataaagcatttttttcactgcattctagttgtggtttgtccaaactcatcaatgtatctta

SEQ ID NO: 16 - DNA, 67 bp, Homo sapiens
  source 1..67: mol_type = genomic DNA
ttcgcatattaaggtgacgcgtgtggcctcgaacaccgagcgaccctgcagcgacccgcttaaaaga

SEQ ID NO: 17 - DNA, 4236 bp, Homo sapiens
  source 1..4236: mol_type = genomic DNA
atggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttga

SEQ ID NO: 18 - DNA, 8807 bp, Homo sapiens
  source 1..8807: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttctagaggcgcgccaagcttggtaccccatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactccgcggccgcactagtgccgccatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgataatagagatctgctagctaataaaagatccttattttcattggatctgtgtgttggttttttgtgtgggatcccgggcccgtcgacgcgccggcgtctagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacagctgcattaatgaatcctgcaggcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctctatgagcggatacatatttgaatgtatttagaaaaataaacaaataggggttccgcgtcatgagattatcaaaaaggatcttcacctagatccttttcacgtagaaagccagtccgcagaaacggtgctgaccccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttcacacaaaaaaccaacacacagatgtaatgaaaataaagatattttattgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgtccattcgccattcaggctgcgcaactgttgggaagggctgcagttaattaactgcagacgccagctg

SEQ ID NO: 19 - DNA, 4577 bp, Homo sapiens
  source 1..4577: mol_type = genomic DNA
atggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactccgcggccgcactagtgccgccatggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatttgataatagagatctgctagctaataaaagatccttattttcattggatctgtgtgttggttttttgtgtg

SEQ ID NO: 20 - DNA, 251 bp, Homo sapiens
  source 1..251: mol_type = genomic DNA
atggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 21 - DNA, 133 bp, Homo sapiens
  source 1..133: mol_type = genomic DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 22 - DNA, 122 bp, Homo sapiens
  source 1..122: mol_type = genomic DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 23 - DNA, 92 bp, Homo sapiens
  source 1..92: mol_type = genomic DNA
agaggtaagggtttaagggatggttggttggtggggtattaatgtttaattacctgttttacaggcctgaaatcacttggttttaggttggg

SEQ ID NO: 24 - DNA, 21 bp, Homo sapiens
  source 1..21: mol_type = genomic DNA
agcttgatatccctgcagggc

SEQ ID NO: 25 - DNA, 93 bp, Homo sapiens
  source 1..93: mol_type = genomic DNA
aagaggtaagggtttaagggatggttggttggtggggtattaatgtttaattacctgttttacaggcctgaaatcacttggttttaggttggg

SEQ ID NO: 26 - DNA, 50 bp, Homo sapiens
  source 1..50: mol_type = genomic DNA
taataaaagatccttattttcattggatctgtgtgttggttttttgtgtg

SEQ ID NO: 27 - DNA, 4233 bp, Homo sapiens
  source 1..4233: mol_type = genomic DNA
atggcgagcatctcagagccggtcacgtttagagagttctgtccactttattaccttcttaatgcaattccaactaaaatccagaaaggatttcgatctatagtagtttatttgaccgcattggatacgaacggcgactacatcgcggttggctcaagcatcggaatgctctatttgtactgtcggcacctcaatcagatgaggaaatataacttcgaaggcaagaccgaatccattacagttgtgaagctcctctcctgtttcgacgacctggtggccgccggcaccgcctccggccgagttgcagtgttccaacttgtatcaagcctccccggcaggaacaagcaactgagaagatttgacgttacgggtattcataagaattccataacggctctggcatggagcccgaacgggatgaaactgttcagcggtgacgataaagggaagatcgtgtactcttctctggatctggaccaagggctttgcaactcccaacttgttctcgaggaaccatcttctatcgtccagcttgactacagtcagaaggtcttgcttgtgtcaacccttcagcgaagccttcttttttatacggaagagaagtccgttcggcagattgggacgcaaccccgcaaaagcaccggtaagtttggcgcatgttttatcccaggactgtgtaaacaatctgacttgacactttatgcctcacgcccaggtctgagactctggaaagccgacgtacacggcactgtacaagctacatttatcttgaaggacgctttcgccggcggcgttaagcccttcgaactgcatcccagattggagtcccccaatagcggttcctgttcccttccagagaggcatctgggtcttgtttcctgtttctttcaggaaggctgggtgctcagctggaacgaatactcaatatacctgcttgatactgtgaaccaagctactgttgcggggctggagggaagcggtgatattgtcagcgtgtcatgcactgaaaatgagattttttttctcaaaggggatcgaaatataattcggatctcatcccggcccgaaggacttactagcactgtaagagacggactggagatgagcggatgttccgaacgcgtgcacgtacaacaggctgagaagctgcccggagctactgtgagtgaaacacggttgcggggctcatccatggcttcttcagttgcaagcgaaccgcgatcacggtcatcatcattgaactcaactgatagcggcagtggactgcttcccccgggattgcaggctacccccgaactcggtaaaggttctcaacctctctctcagcgcttcaacgcgatctcctcagaagatttcgaccaggagttggttgttaagcccatcaaagtcaagcgcaaaaagaaaaaaaaaaagaccgaagggggctcccggagtacgtgccactcctctcttgagagtactccgtgtagtgagtttccaggcgacagtccgcagtctctgaacacagaccttttgagtatgacaagctcagtgcttgggagcagtgtagaccaactttcagctgagtcaccggatcaggagtcttctttcaatggcgaggttaatggtgtaccacaggagaatacagatcccgaaacattcaacgtcctcgaagtctctggtagtatgccagattccctcgctgaagaagatgacatacggacggaaatgccccattgccaccacgcgcatggtcgagagcttcttaacggagcaagggaagacgtagggggctccgacgtaacggggttgggtgatgaaccgtgcccggctgacgacggtcccaatagtacacagttgccgttccaagaacaggacagttcccccggcgcgcatgatggagaggacatccagccgataggtccacaaagtactttctgcgaggttccacttctcaactcactcactgtcccgagctctctttcatgggccccgtcagcagaacaatggttgccaggaacgcgcgctgatgaaggctccccggttgaaccatcccaagagcaggacatactgacatccatggaagctagtgggcacctttctactaatctgtggcacgcggtcacggatgacgatacgggtcagaaggagataccaatctccgagcgcgtactcggctccgtaggcggacaacttactcccgtatctgcgctcgcagccagtacccacaagccatggctcgagcagccacctcgagatcaaactctgacgtcatcagatgaagaggacatttacgcccacgggctgccctcttcttcctcagagacatccgttacagagttgggtccgtcttgctcacaacaagatttgtcaagacttggtgccgaagatgctggtctcctgaaacctgatcagtttgcagagtcctggatgggctactctggcccgggctatgggatactctccctggtcgtctccgaaaagtatatttggtgtcttgattataaggggggtctcttctgttctgcattgccgggagcaggcttgagatggcaaaaattcgaggatgcagtacaacaagtagccgtatcacctagtggagcattgctgtggaagattgagcaaaaatcaaatcgggcgtttgcttgtggaaaggtaacgattaaaggtaagcgacactggtacgaagcacttccccaggccgtatttgttgcgctgtccgatgacaccgcctggatcattcgaacatctggggatttgtatcttcaaacggggctcagcgtagatagaccttgtgctcgggcggtaaaagtggactgcccataccccttgtcacagattactgcacgaaacaacgtcgtttgggctctgacggagcaacgagctctgctctatagggaaggtgtaagctctttctgcccagaaggggaacagtggaagtgtgatatcgtatcagagcgccaagctctcgaacccgtatgtatcacactgggtgatcagcaaacgctttgggctcttgacatacatggaaatctttggtttcgcacaggcatcatttcaaaaaaaccacagggagacgacgaccactggtggcaagtttctatcacggattatgtcgtattcgatcagtgttcacttttccagaccataatccatgccacgcattccgtggcaaccgcggctcaagctcccgtagaaaaggtggcagataaactccgcatggcattttggtcacaacagcttcagtgccaaccttctttgcttggtgtgaataactctggagtttggatttccagtggtaaaaacgaattccacgttgctaagggcagccttattggtacttattggaatcatgttgtccctagaggaacagcctctgcgaccaagtgggccttcgtacttgcatcagcggcgccaactaaggagggttcattcctctggctttgccagtctagtaaagacctctgtagtgtgtccgcacaaagcgcccaaagccgaccaagcaccgtgcagctgccgccggaggccgagatgagggcgtacgccgcatgtcaagacgctttgtgggcgcttgattctctgggtcaggtatttattaggactctttcaaagagctgccccacgggcatgcactggacgcggctggatctctctcagcttggggccgtaaagctcacttctcttgcgtgtggcaaccagcatatctgggcatgcgattccagagggggagtatactttcgagtgggcacacaacctcttaatccgtcactgatgcttccagcgtggataatgatcgagccgccggtccaacccgcgggtgtttcacttgttagtgttcattcatctccgaatgaccaaatgctctgggtcctcgactctagatggaatgttcacgtacggacgggtattacagaggagatgcctgtaggtacggcttgggagcatgtccctggcctgcaggcatgtcaactggctctgtccactaggacggtctgggccaggtgtccaaatggggaccttgccagacggtacggagtgacagacaagaatcctgcaggtgactattggaagaaaatacccggtagtgtaagctgtttcaccgtcacggcctcagatgaactgtgggctgtgggaccacctggttacctcctccagcgcttgactaagacattctcccattcccacggcacccagaaaagttcacaagctgctatgccacatccagaagacctggaagatgagtgggaagttatt

SEQ ID NO: 28 - protein, 286 aa, Homo sapiens
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 29 - protein, 264 aa, Homo sapiens
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

(19) United States
(12) Patent Application Publication
(10) Pub. No.: US 2024/0024512 A1
(43) Pub. Date: Jan. 25, 2024

(54) METHODS AND COMPOSITIONS FOR TREATING TECPR2-ASSOCIATED DISEASE AND DISORDERS WITH A VIRAL VECTOR

(71) Applicant: University of Florida Research Foundation, Incorporated, Gainesville, FL (US)

(72) Inventors: Barry John Byrne, Gainesville, FL (US); Manuela Corti, Gainesville, FL (US); Shandra Trantham, Gainesville, FL (US)

(73) Assignee: University of Florida Research Foundation, Incorporated, Gainesville, FL (US)

(21) Appl. No.: 18/349,129

(22) Filed: Jul. 8, 2023

Related U.S. Application Data
(60) Provisional application No. 63/359,731, filed on Jul. 8, 2022.

(51) Int. Cl. A61K 48/00; C12N 15/86; C07K 14/47

(52) U.S. Cl. CPC A61K 48/0058; C12N 15/86; C07K 14/47; C12N 2750/14143; C12N 2750/14171

(57) ABSTRACT

The present disclosure relates to compositions and methods for the treatment of TECPR2-associated diseases or disorders. Several embodiments provided for herein relate to virally-mediated transfer of a gene to target cells to induce expression of an encoded polypeptide, protein or other product in order to ameliorate one or more symptoms of a TECPR2-associated disease or disorder in a subject. In several embodiments, the disclosed methods and compositions relate to recombinant adeno-associated virus particles encoding human TECPR2 in order to treat TECPR2-associated diseases or disorders, including spastic paraplegic type 49 (SPG49) and/or hereditary sensory and autonomic neuropathy 9 (HSAN9).

### RELATED APPLICATIONS

The application claims the benefit under 35 U.S.C. 119(e) of U.S. Provisional Application No. 63/359,731 filed Jul. 8, 2022, which is incorporated by reference herein in its entirety.

### REFERENCE TO AN ELECTRONIC SEQUENCE LISTING

The contents of the electronic sequence listing (U120270097US01-SEQ-PRW.xml; Size: 121,770 bytes; and Date of Creation: Jul. 7, 2023) is herein incorporated by reference in its entirety.

### BACKGROUND

Tectonin beta-propeller repeat containing 2 (TECPR2) is a gene involved in cellular autophagy. In the autophagy pathway, TECPR2 regulates targeting of autophagosomes to lysosomes. Mutations in TECPR2 disrupt key processes in the autophagy pathway. Certain inherited disorders, such as spastic paraplegia type 49 (SPG49) and hereditary sensory and autonomic type IX (HSAN9), lead to neuropathies and involve abnormal autophagy mechanisms in nervous system tissue.

### SUMMARY

Inherited disorders giving rise to neuropathies confer an array of abnormalities in affected individuals that range from mild symptoms to potential lethality. Inherited disorders may comprise neuropathies which are caused by progressive degeneration and death of peripheral sensory neurons. In many cases, options for treatment of neuropathies are limited.

Spastic paraplegia type 49 (SPG49) is a severe neurodegenerative disorder that is part of a large group of genetic disorders known as hereditary spastic paraplegias. Characteristics of SPG49 include, but are not limited to, abnormal muscle spasticity, limb paralysis, intellectual disability, short stature, microcephaly, brachycephaly, shortened and thickened neck structure, craniofacial abnormalities, and seizures. Certain autonomic nervous system functions are impaired in SPG49 patients, including heart rate, digestion, and breathing, which can lead to complications that require intense medical intervention and, in extreme cases, lead to early mortality. In some instances, abnormalities associated with SPG49 are similar to those seen in hereditary sensory and autonomic neuropathy (HSAN), which is a group of conditions that affect sensory and autonomic neurons. SPG49 is thought to be related to a specific HSAN called HSAN9.

Further linking SPG49 and HSAN9 is mutation in the tectonin-beta propeller repeat containing 2 (TECPR2) gene. TECPR2 is a multi-domain protein comprised of three tryptophan-aspartic acid (WD) repeats at the N-terminus. TECPR2 further comprises a microtubule associated protein 1 light chain 3 beta (LC3)-interacting region at its C-terminus. An unstructured region spans the middle of the TECPR2 protein thereby separating the N- and C-terminal domains.

TECPR2 is highly expressed in nervous system tissues, in which this protein plays a role in the autophagy pathway by regulating the formation of autophagosomes. Impaired autophagy results from mutation of TECPR2, resulting in altered function of axons and dendrites. Fibroblast cell line models and mouse models of TECPR2 mutation exhibit accumulation of autophagosomes indicating that TECPR2 facilitates targeting of autophagosomes to lysosomes.

This disclosure is based, at least in part, on the generation of viral vectors adapted for delivery of a TECPR2-encoding nucleic acid to mammalian tissues and robust expression of TECPR2 in these tissues. Disclosed herein are gene delivery approaches for treatment of human subjects with one or more diseases or disorders associated with a TECPR2 mutation.

Accordingly, some aspects of the present disclosure provide a nucleic acid comprising a TECPR2 sequence and flanked on each side by inverted terminal repeat sequences. In some embodiments, the nucleic acid comprises a TECPR2 sequence operably linked to one or more regulatory elements. In some embodiments, the TECPR2 sequence is a human TECPR2 sequence. In some embodiments, the TECPR2 sequence is codon-optimized.

In some embodiments, the present disclosure provides recombinant adeno-associated virus (rAAV) vectors for delivering transgenes into the nervous system tissues of a subject. Such rAAV vectors may include, from 5′ to 3′, in order, a first adeno-associated virus (AAV) inverted terminal repeat (ITR) sequence, a promoter operably linked to one or more transgenes, and a second AAV inverted terminal repeat (ITR) sequence. In some embodiments, the rAAV vector includes, in addition to a promoter, a regulatory element which modifies expression, e.g., in a manner that provides physiologically relevant expression levels and/or restricts expression to a particular cell type or tissue. In some embodiments, the regulatory element comprises one or more of an enhancer, a 5′ untranslated region (UTR), and a 3′ UTR.

In some embodiments, the rAAV vector also includes at least one polyadenylation (polyA) signal (e.g., positioned 3′ of the one or more transgenes, such as one that is operably linked to a transgene). In some embodiments, two transgenes are operably linked to the same single promoter. In some embodiments, each transgene is operably linked to a separate promoter. In some embodiments in which multiple transgenes are provided, the rAAV vector includes at least one polyadenylation signal operably linked to a transgene (e.g., positioned 3′ of two transgenes expressed from a single promoter or 3′ of one or both transgenes expressed from different promoters). Aspects of the disclosure provide recombinant adeno-associated virus (rAAV) nucleic acid vectors for delivering two or more transgenes into the nervous system tissues of a subject, wherein said vector comprises, from 5′ to 3′, a first adeno-associated virus (AAV) inverted terminal repeat (ITR) sequence, two or more transgenes and a promoter operably linked to the two or more transgenes, a polyadenylation signal, and a second AAV inverted terminal repeat (ITR) sequence.

In some embodiments, the transgene is a TECPR2 gene. In some embodiments, the transgene is a TECPR2 gene that includes non-coding sequences, such as introns. In some embodiments, the transgene is a TECPR2 cDNA, such as a human TECPR2 (hTECPR2) cDNA. In some embodiments, the transgene comprises a series of exons of human TECPR2 gene arranged in series (or in sequence). In some embodiments, the transgene is encoded by a polynucleotide having at least 85%, at least 90%, at least 95%, at least 96%, at least 97%, at least 98%, or at least 99% sequence identity to a nucleotide sequence in any one of SEQ ID NOs: 1-5 or 7-27 (e.g., SEQ ID NOs: 17 or 27, or a transgene encoding a protein comprising the amino acid sequence of SEQ ID NO: 6 that is encoded by a corresponding nucleic acid that may differ from any of SEQ ID NOs: 1-5 or 7-27). In some embodiments, the transgene is encoded by a polynucleotide comprising at least a portion, or the entire sequence, of any of the sequences set forth in SEQ ID NOs: 1-5 or 7-27 (e.g., a transgene comprising the sequence set forth in SEQ ID NOs: 17 or 27). In some embodiments, the transgene contains a nucleic acid sequence that differs from any of the sequences set forth as SEQ ID NOs: 1-5 or 7-27 by 1, 2, 3, 4, 5, 6, 7, 8, 9, 10, 10-15, 15-20, 20-25, 25-35, 35-45, 55-65, 65-75, 75-100, 100-150, 150-200, or more than 200 nucleotides (e.g., a transgene comprising the sequence set forth in SEQ ID NO: 17 or 27 but not comprising other sequences provided in other nucleic acids described herein, such as sequences in SEQ ID NOs: 1-5 or 18 that do not encode SEQ ID NO: 17 or 27). In some embodiments, one or more of the transgenes of the present disclosure are naturally-occurring sequences that are not normally found in recombinant viruses (e.g., recombinant AAVs). In some embodiments, one or more transgenes are engineered to be species-specific. In some embodiments, one or more transgenes are codon-optimized for expression in a species of interest, e.g., a mammalian species, such as a human. For example, in several embodiments, the transgene (e.g., a TECPR2 transgene) is codon-optimized. In some embodiments, the transgene (e.g., one comprising a TECPR2 sequence) is, upon expression in a cell (e.g., a cell in a subject comprising a TECPR2 mutation), useful in treating a TECPR2-associated disease or disorder.

Further provided herein are rAAV particles containing any of the rAAV vectors disclosed herein, encapsidated in an AAV capsid comprising at least one AAV capsid protein. Other aspects of the present disclosure include compositions containing any of the nucleic acids or the rAAV particles described herein. In several embodiments, such compositions may be administered to a subject for gene therapy for a TECPR2-associated disease or disorder, for example one or more TECPR2-associated diseases or disorders including spastic paraplegia type 49 (SPG49) and hereditary sensory and autonomic neuropathy type IX (HSAN9). In some embodiments, the TECPR2-associated disease or disorder causes intellectual disability, spastic ataxic gait, and autonomic dysfunction in the subject.

The compositions of the present disclosure may be administered to the subject via one or more of several different routes. In some embodiments, the composition is administered via intravenous (IV) injection into the subject. In some embodiments, the administration of the composition results in expression of the transgene (or, if multiple transgenes are used, expression of two or more transgenes) in the subject's nervous system tissue. In some embodiments, the composition is administered via injection into the cerebrospinal fluid of a subject. In various embodiments, the step of administering the composition results in improvements in one or more symptoms associated with SPG49 or HSAN9 in a subject for more than 12 months, more than 14 months, more than 16 months, more than 17 months, more than 20 months, more than 22 months, or more than 24 months.

In some embodiments, described herein are compositions comprising AAV vectors, virions, viral particles, and pharmaceutical formulations thereof, useful in methods for delivering genetic material encoding one or more beneficial or therapeutic product(s) to mammalian cells and tissues. Any of the rAAV vectors, rAAV particles, or compositions comprising the rAAV particles of the present disclosure, may be used for gene therapy for treatment of one or more TECPR2-associated diseases or disorders, including but not limited to SPG49 and HSAN9. Any of the rAAV vectors, rAAV particles, or compositions comprising the rAAV particles of the present disclosure may be administered to a subject in need thereof, such as a human subject suffering from a TECPR2-associated disease or disorder, such as SPG49 or HSAN9.

Additionally, provided herein are compositions, as well as therapeutic and/or diagnostic kits that include one or more of the disclosed AAV compositions, formulated with one or more additional ingredients, or prepared with one or more instructions for their use.

In some embodiments, described herein is a nucleic acid comprising a TECPR2 sequence. In some embodiments, a nucleic acid comprising a TECPR2 sequence further comprises one or more regulatory elements. In some embodiments, the one or more regulatory elements is a promoter. In some embodiments, a nucleic acid comprises a human TECPR2 sequence and an enhancer element, such as a CMV enhancer, operably linked to a promoter, wherein the TECPR2 sequence and its operably linked sequences are flanked on each side by an inverted terminal repeat sequence (ITR). In some embodiments, the human TECPR2 sequence is codon-optimized for expression in human cells. In some embodiments, the promoter comprises a neuron-specific promoter. In some embodiments, the promoter is derived from the TECPR2 gene.

In some embodiments, a method of treating SPG49 and/HSAN9 is described, the method comprising administering a therapeutically effective amount of rAAV particle comprising a nucleic acid comprising a human TECPR2 sequence operably linked to a promoter and a polyA signal and optionally an enhancer element, wherein the TECPR2 sequence and its operably linked sequences are flanked on each side by an inverted terminal repeat sequence, and wherein said administration results in expression of a therapeutically effective amount of human TECPR2, thereby treating a TECPR2-associated disease or disorder. In some embodiments, the rAAV is administered via intravenous injection. In some embodiments, the rAAV is administered via injection into the cerebrospinal fluid.

In some embodiments, a method of treating a TECPR2-associated disease or disorder is described, the method comprising administering a therapeutically effective amount of rAAV particle comprising a nucleic acid comprising a human TECPR2 sequence operably linked to a promoter and optionally an enhancer element and/or a polyA signal, wherein the TECPR2 sequence and its operably linked sequences are flanked on each side by an inverted terminal repeat sequence, and wherein said administration results in expression of a therapeutically effective amount of human TECPR2, thereby treating a TECPR2-associated disease or disorder. In some embodiments of the disclosed methods, a therapeutically effective amount of rAAV comprising a nucleic acid described herein is administered to a subject (e.g., a human) to treat intellectual disability, spastic ataxic gait, and autonomic dysfunction in the subject.

In some embodiments, a method of treating a TECPR2-associated disease or disorder is described, the method comprising administering a therapeutically effective amounts of an rAAV comprising a nucleic acid comprising a human TECPR2 sequence operably linked to a promoter and optionally an enhancer element, wherein the nucleic acid further comprises an operably linked polyA signal and is flanked on each side by an inverted terminal repeat sequence, and wherein said administration results in expression of a therapeutically effective amount of human TECPR2, thereby treating a TECPR2-associated disease or disorder.

In some embodiments, the rAAV particle, e.g., comprising a TECPR2 coding sequence is administered via intravenous injection. In some embodiments, between about 1×10¹³ and about 1×10¹⁴ rAAV vector genomes (vg) are administered. In some embodiments, at least 20%, at least 30%, at least 40%, or at least 50% of nervous system tissue cells are transduced when the rAAV vector genomes are administered. In some embodiments, at least 20%, at least 30%, at least 40%, or at least 50% of nervous system tissue cells are transduced when between about 1×10¹³ and about 1×10¹⁴ rAAV vector genomes are administered.

Also described herein is a method of increasing expression of human TECPR2 in a target cell, comprising contacting a target cell (such as a cell of the nervous system) with a plurality of rAAV particles comprising a nucleic acid comprising a functional human TECPR2 sequence and optionally an enhancer element operably linked to a promoter, wherein the nucleic acid further comprises a polyA signal operably linked to the TECPR2 sequence and is flanked on each side by an inverted terminal repeat sequence, and wherein said contacting results in the target cell increasing expression of functional human TECPR2 as compared to prior to the contacting, thereby increasing the expression of functional human TECPR2.

Also described herein is a method of increasing expression of human TECPR2 in a target cell (such as a cell of the nervous system), comprising contacting a target cell with a plurality of rAAV particles comprising a nucleic acid comprising a functional human TECPR2 sequence operably linked to a promoter and optionally an enhancer element, wherein the nucleic acid further comprises a polyA signal operably linked to the TECPR2 sequence and is flanked on each side by an inverted terminal repeat sequence, and wherein said contacting results in the target cell increasing expression of functional human TECPR2 as compared to prior to the contacting, thereby increasing the expression of functional human TECPR2.

Also described herein is a method of increasing expression of human TECPR2 in a target cell (e.g., a cell of the nervous system), comprising contacting a target cell (e.g., a cell of the nervous system) with a plurality of rAAV particles, wherein the rAAV particles comprise a nucleic acid comprising a functional human TECPR2 sequence operably linked to a promoter and optionally an enhancer element, wherein the TECPR2 sequences and its operably linked sequences is flanked on each side by an inverted terminal repeat sequence, and wherein said contacting results in the target cell (e.g., a cell of the nervous system) increasing expression of functional human TECPR2 as compared to prior to the contacting, thereby increasing the expression of functional human TECPR2.

In some embodiments, the contacting is in vivo. In some embodiments, the method is used for the treatment of a TECPR2-associated disease or disorder such as SPG49 and/or HSAN9. In some embodiments, the method is used for the treatment of intellectual disability, spastic ataxic gait, and/or autonomic dysfunction associated with a TECPR2-associated disease or disorder. In some embodiments, the nucleic acids, the rAAV particles, the compositions, or the methods of manufacture described herein can be used for the treatment of a TECPR2-associated disease or disorder, such as SPG49 and/or HSAN9. In some embodiments, the nucleic acids, the rAAV particles, the compositions, or the methods of manufacture described herein can be used for the treatment of intellectual disability, spastic ataxic gait, and/or autonomic dysfunction associated with a TECPR2-associated disease or disorder.

Further provided herein are uses of any of the disclosed nucleic acids, rAAV particles, or compositions for the treatment of a TECPR2-associated disease or disorder, or in the manufacture of a medicament for the treatment of a TECPR2-associated disease or disorder. Accordingly, in some embodiments, nucleic acids provided herein may comprise an expression construct having a TECPR2 sequence (e.g., one comprising or consisting of a coding sequence for a TECPR2 protein) operably linked to one or more promoters, polyA signals, and/or other regulatory elements.

### BRIEF DESCRIPTION OF THE DRAWINGS

FIGS. 1A-1B show a non-limiting example of a nucleic acid embodiment disclosed herein. FIG. 1A shows a sequence map for Construct 1 (corresponding to SEQ ID NO: 1).

FIG. 1B shows the sequence annotations of Construct 1, including sequences encoding the AmpR sequence (SEQ ID NO: 28) and NeoR/KanR sequence (SEQ ID NO: 29)

FIGS. 2A-2B show a second non-limiting example of a nucleic acid embodiment disclosed herein. FIG. 2A shows a sequence map for Construct 2 (corresponding to SEQ ID NO: 2). FIG. 2B shows the sequence annotations of Construct 2, including sequences encoding the AmpR sequence (SEQ ID NO: 28) and NeoR/KanR sequence (SEQ ID NO: 29).

FIGS. 3A-3B show a third non-limiting example of a nucleic acid embodiment disclosed herein. FIG. 3A shows a sequence map for Construct 3 (corresponding to SEQ ID NO: 3). FIG. 3B shows the sequence annotations of Construct 3, including sequences encoding the AmpR sequence (SEQ ID NO: 28) and NeoR/KanR sequence (SEQ ID NO: 29).

FIGS. 4A-4B show a fourth non-limiting example of a nucleic acid embodiment disclosed herein. FIG. 4A shows a sequence map for Construct 4 (corresponding to SEQ ID NO: 4). FIG. 4B shows the sequence annotations of Construct 4, including sequences encoding the AmpR sequence (SEQ ID NO: 28) and NeoR/KanR sequence (SEQ ID NO: 29).

FIGS. 5A-5B show a fifth non-limiting example of a nucleic acid embodiment disclosed herein. FIG. 5A shows a sequence map for Construct 5 (corresponding to SEQ ID NO: 5). FIG. 5B shows the sequence annotations of Construct 5, including sequences encoding the AmpR sequence (SEQ ID NO: 28) and NeoR/KanR sequence (SEQ ID NO: 29).

FIG. 6 shows the degree of hTECPR2 expression (-fold) change following HEK293T transfection with each gene therapy construct described in FIGS. 1-5 relative to non-transfected control, as determined by quantitative PCR.

FIGS. 7A-7B show a sixth non-limiting example of a nucleic acid embodiment described herein. FIG. 7A shows a sequence map for Construct 6 (corresponding to SEQ ID NO: 18). FIG. 7B shows the sequence annotations of Construct 6, including sequences encoding the AmpR sequence (SEQ ID NO: 28) and NeoR/KanR sequence (SEQ ID NO: 29).

### DETAILED DESCRIPTION

Reference is made to particular features and/or non-limiting embodiments of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, alone or in combination with and/or in the context of other particular aspects and embodiments of the invention, and in the invention generally.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

A “TECPR2-associated disease or disorder” refers to any disease, disorder, symptom, or medical condition that arises as a result of a dysfunctional TECPR2 gene and/or TECPR2 protein. Examples of dysfunctional TECPR2 genes are TECPR2 genes that comprise one or more substitutions, insertions, or deletions relative to a wild type TECPR2 gene (or mRNA encoded by a wild type TECPR2 gene), and may be referred to as a “mutant” TECPR2 or a TECPR2 variant. The number of such mutations in a TECPR2 variant may vary. In some embodiments, a TECPR2-associated disease or disorder may arise due to deletions and/or inclusions of entire exons and/or introns in the TECPR2 mRNA sequence such that an improper splice variant is produced. In some embodiments, a TECPR2-associated disease or disorder may arise due to mutation of a TECPR2 regulatory sequence (e.g., in the natural TECPR2 promoter, transcription start site, transcription stop site, translation start site, stop codon, etc.). In some embodiments, a TECPR2-associated disease or disorder arises from altered transcription levels of the TECPR2 gene and/or altered translation levels of TECPR2 protein. Here, “altered” refers to any biological pathway (e.g., transcription and translation) that is being catalyzed at a rate that is either increased or decreased at a given time relative to a wildtype cell. In some embodiments, dysfunction of the TECPR2 protein through circumstances, such as altered post-translational modification, altered binding activity, and/or altered activity of a regulator upstream of TECPR2 thereby affecting its function may also give rise to a TECPR2-associated disease or disorder. Examples of TECPR2-associated diseases or disorders include, but are not limited to, spastic paraplegia type 49 (SPG49) and hereditary sensory and autonomic neuropathy type IX (HSAN49).

A “subject” refers to mammal that is the object of treatment using a method or composition as provided for herein. “Mammal” includes, without limitation, mice, rats, rabbits, guinea pigs, dogs, cats, sheep, goats, cows, horses, primates, such as monkeys, chimpanzees, and apes, and humans. In some embodiments, the subject is human.

The terms “treating,” “treatment,” “therapeutic,” or “therapy” do not necessarily mean total cure or abolition of the disease or condition. Any alleviation of any undesired signs or symptoms of a disease or condition, to any extent (e.g., to any statistically significant and/or clinically significant extent) can be considered treatment and/or therapy. To “treat” a disease as the term is used herein, means to reduce the frequency or severity of at least one sign or symptom of a disease or disorder experienced by a subject.

The term “effective amount,” as used herein, refers to an amount that is capable of treating or ameliorating a disease or condition or otherwise capable of producing an intended therapeutic effect, such as reducing the frequency or severity of at least one sign or symptom of a disease or disorder experienced by a subject.

A “nucleic acid” sequence refers to a deoxyribonucleic acid (DNA), ribonucleic acid (RNA) sequence, or a combination thereof (e.g., one that comprises both DNA and RNA nucleotides). This term also encompasses naturally-occurring and non-naturally occurring nucleobases (bases). For example, this term encompasses sequences that include any of the known base analogues of DNA and RNA, such as but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “polynucleotide,” refers to a polymeric form of nucleotides of any length, including DNA, RNA, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

For the purpose of describing the relative position of nucleotide sequences in a particular nucleic acid molecule throughout the instant application, such as when a particular nucleotide sequence is described as being situated “upstream,” “downstream,” “3′,” or “5′” relative to another sequence, it is to be understood that it is the position of the sequences in the “sense” or “coding” strand of a DNA molecule that is being referred to as is conventional in the art.

The term “isolated” when referring to a nucleotide sequence, means that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not materially affect the basic characteristics of the composition.

As used herein, the term “variant” refers to a molecule having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild-type counterpart. Variants of a protein molecule may contain modifications to the sequence (e.g., having 1, 2, 3, 4, 5, 6, 7, 8, 9, 10, 10-15, or 15-20 amino acid substitutions) relative to the wild-type sequence. These modifications include chemical modifications as well as truncations.

The term “identity” refers to a nucleotide-to-nucleotide or amino acid-to-amino acid correspondence of two polynucleotides or polypeptide sequences, respectively. Two or more sequences (polynucleotide or amino acid) can be compared by determining their “percent identity.” The “percent (%) identity” of two sequences, whether nucleic acid or amino acid sequences, is the number of exact matches between two aligned sequences divided by the length of the shorter sequences and multiplied by 100. This term may refer to the extent to which two sequences (nucleotide or amino acid) have the same residue at the same positions in an alignment. For example, “an amino acid sequence is X % identical to SEQ ID NO: 6” refers to % identity of the amino acid sequence to SEQ ID NO: 6 and is elaborated as X % of residues in the amino acid sequence are identical to the residues of sequence disclosed in SEQ ID NO: 6. Generally, computer programs are employed for such calculations. In some instances, such computer programs may introduce gaps into sequences for the purposes of alignment and determining identity between sequences.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “recombinant,” as applied to a polynucleotide (e.g., one that may serve as a viral genome) means that the polynucleotide is the product of one or more of cloning, restriction or ligation steps, and other procedures (e.g., PCR, such as mutagenic PCR) that result in a nucleic acid (e.g., also referred to as a nucleic acid construct or an expression construct) that is distinct from a polynucleotide found in nature. Accordingly, a recombinant virus is a viral particle comprising a recombinant polynucleotide (e.g., a recombinant AAV (rAAV) genome comprising a nucleic acid that is heterologous to the inverted terminal repeats which flank the heterologous nucleic acid).

The term “gene,” refers to a polynucleotide containing at least one open reading frame that is capable of encoding a particular gene product (e.g., an RNA, such as an mRNA). Any of the polynucleotide sequences described herein may be used to identify larger fragments or full-length coding sequences of the genes with which they are associated. Methods of isolating larger fragment sequences are known to those of skill in the art.

The term “transgene,” as used herein, refers to a nucleic acid sequence to be positioned within a viral vector and encoding a polypeptide, protein, or other product (e.g., an RNA, such as RNA that does not encode a protein) of interest. In some embodiments, one rAAV vector may comprise a sequence encoding one or more transgenes (which can optionally be the same gene, or different genes). For example, one rAAV vector may comprise the sequence for 1, 2, 3, 4, 5, 6, 7, 8, 9, or 10 transgenes. The transgenes of the present disclosure relate to the improvement of one or more diseases or disorders associated with TECPR2 including, but not limited to, SPG49 and/or HSAN9.

The terms “gene transfer” or “gene delivery” refer to methods or systems for inserting DNA, such as a transgene, into cells, such as those of a subject afflicted with a TECPR2-associated disease or disorder. In several embodiments, gene transfer yields transient expression of non-integrated transferred DNA, extrachromosomal replication, and expression of transferred replicons (e.g., episomes). In additional embodiments, gene transfer results in integration of transferred genetic material into the genomic DNA of target cells or host cells. Examples of host cells of the disclosure include HEK293 and C2C12 myoblast cells. Further examples of host cells include those comprising a mutation that gives rise to a TECPR2-associated disease or disorder (e.g., a TECPR2 mutation or a mutation that gives rise to SP49 and/or HSAN9). In some embodiments, host cells may be in a subject having, suspected of having, or at risk of developing a TECPR2-associated condition. Alternatively, such host cells may be obtained from the subject (e.g., contacted ex vivo with a nucleic acid described herein, optionally wherein the cells are then administered to the subject after contacting). Additional exemplary host cells of the disclosure include human cells derived from an induced pluripotent stem cell or a neuronal cell line.

The terms “regulatory element” or “regulatory sequence”, or variations thereof, refer to a nucleotide sequence that participates in functional regulation of a nucleic acid, including replication, duplication, transcription, splicing, translation, or degradation of the nucleic acid. Regulatory elements can be enhancing or inhibitory in nature, depending on the embodiment. Non-limiting examples of regulatory elements include transcriptional regulatory sequences, such as promoter sequences, polyadenylation (polyA) signals, transcription termination sequences, upstream regulatory domains, origins of replication, internal ribosome entry sites (“IRES”), enhancers, and the like. In some embodiments, any of the disclosed rAAV vectors comprise an IRES. In some embodiments, any of the disclosed rAAV vectors comprise a splice donor/splice acceptor (SD/SA) sequence, such as an endogenous SD/SA sequence. These elements collectively provide for the replication, transcription, and translation of a coding sequence in a recipient cell, though not all of these sequences need always be present. It shall be appreciated that the structural components of a rAAV vector as provided for herein may be listed in individual paragraphs solely for clarity and may be used together in combination. For example, any regulatory element or other component can be used in combination with any nucleic acid comprising a TECPR2 sequence (e.g., transgene (or transgenes), an expression cassette, a vector, etc.) provided for herein.

A “promoter” is a polynucleotide that interacts with an RNA polymerase and initiates transcription of a coding region (e.g., a transgene) usually located downstream (in the 3′ direction) from the promoter.

The term “operably linked” refers to an arrangement of elements wherein the components are configured to perform a function. For example, regulatory elements (e.g., a promoter) operably linked to nucleic acid (e.g., a DNA molecule) may result in the expression (e.g., transcription) of a sequence (e.g., a coding sequence) encoded therein. In a further example, a nucleic acid may be operably linked to a regulatory element comprising a sequence that, upon expression, results in degradation of an RNA encoded by the nucleic acid. Depending on the embodiment, a regulatory sequence need not be contiguous with the sequence. Thus, for example, one or more sequences that are typically do not get translated (e.g., an intron), yet are capable of being transcribed, can be present between a promoter sequence and a sequence, with those two sequences still being considered “operably linked”.

The term “vector” means any molecular vehicle, such as a plasmid, phage, transposon, cosmid, chromosome, virus, viral particle, virion, etc. which can transfer nucleic acids (e.g., a transgene) to or between cells of interest.

An “expression vector” is a vector comprising a region of nucleic acid (e.g., a transgene) which encodes a gene product (e.g., an RNA, such as one encoding a polypeptide or protein) of interest. As disclosed herein, vectors are used for achieving expression, e.g., stable expression, of a protein in an intended target cell (e.g., a cell of the nervous system). An expression vector may also comprise control elements operably linked to the transgene to facilitate expression of the protein encoded therein in the target cell (e.g., a cell of the nervous system).

An “expression cassette” refers to a combination of one or more regulatory elements and a nucleic acid sequence or sequences to which they are operably linked for expression.

The term “AAV” is an abbreviation for adeno-associated virus and may be used to refer to the virus itself or derivatives thereof. The term covers all subtypes and both naturally occurring and recombinant forms, unless otherwise indicated. The abbreviation “rAAV” refers to recombinant adeno-associated virus, also referred to as a recombinant AAV vector (or “rAAV vector”), which refers to AAV comprising a polynucleotide sequence not of AAV origin (e.g., a transgene). The term “AAV” includes AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), serotype rh10 AAV, serotype rh74 AAV, or a pseudotyped rAAV (e.g., AAV2/9, referring an AAV vector with the genome of AAV2 (e.g., the ITRs of AAV2) and the capsid of AAV9). In several embodiments, the preferred serotype for delivery to human patients affected by a TECPR2-associated disease or disorder is one of AAV-9, serotype rh74, serotype rh10, or AAV-8. However, other AAV serotypes may be used as the term “AAV” is not limited to any particular serotype or variant.

The term “AAV virus” or “AAV viral particle” or “rAAV vector particle” refers to a viral particle comprise at least one AAV capsid protein and an encapsidated nucleic acid (e.g., a recombinant genome comprising a nucleic acid that is heterologous to the inverted terminal repeats which flank it (e.g., not normally found in nature between inverted terminal repeats)).

The term “heterologous” refers to genotypically distinct origins. For example, a heterologous polynucleotide is one derived from a different species or gene as compared to a reference species or gene (for example a human gene inserted into a viral plasmid is a heterologous gene). A promoter removed from its native coding sequence and operably linked to a sequence with which it is not naturally found linked is a heterologous promoter.

As used herein, the term “kit” may be used to describe variations of the portable, self-contained enclosure that includes at least one set of components to conduct one or more of the diagnostic or therapeutic methods of the present disclosure.

The term “carrier” refers to a diluent, adjuvant, excipient, or vehicle with which the rAAV particle or preparation, and/or rAAV vectors is administered. Such pharmaceutical carriers can be sterile liquids, such as water and oils, including those of petroleum oil, such as mineral oil, vegetable oil, such as peanut oil, soybean oil, and sesame oil, animal oil, or oil of synthetic origin. Saline solutions and aqueous dextrose and glycerol solutions may also be employed as liquid carriers.

Terms and phrases used in this application, and variations thereof, including in the appended claims, unless otherwise expressly stated, should be construed as open ended as opposed to limiting. As examples of the foregoing, the term “including” should be read to mean ‘including, without limitation,’ ‘including but not limited to,’ or the like; the term “comprising” as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term “includes” should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

The ranges disclosed herein also encompass any and all overlap, sub-ranges, and combinations thereof. Language, such as “up to,” “at least,” “greater than,” “less than,” “between,” and the like includes the number recited. Numbers preceded by a term, such as “about” or “approximately” include the recited numbers. For example, “about 90%” includes “90%.” In some embodiments, at least 95% homologous or identical includes 96%, 97%, 98%, 99%, and 100% homologous or identical to the reference sequence. In addition, when a sequence is disclosed as “comprising” a nucleotide or amino acid sequence, such a reference shall also include, unless otherwise indicated, that the sequence “consists of” or “consists essentially of” the recited sequence. Likewise, when a composition is disclosed as “comprising” a feature, such a reference shall also include, unless otherwise indicated, that the composition “consists of” or “consists essentially of” the recited feature.

TECPR2-Associated Diseases or Disorders

In some embodiments, disclosed herein are gene delivery approaches for treatment of human subjects with one or more diseases or disorders associated with TECPR2 mutation.

Spastic paraplegia type 49 (SPG49) is a severe neurodegenerative disorder that is part of a large group of genetic disorders known as hereditary spastic paraplegias. Characteristics of SPG49 include, but are not limited to, abnormal muscle spasticity, limb paralysis, intellectual disability, short stature, microcephaly, brachycephaly, shortened and thickened neck structure, craniofacial abnormalities, and seizures. Additional autonomic nervous system functions are impaired in SPG49 including heart rate, digestion, and breathing which can lead to complications that require intense medical intervention and, in extreme cases, lead to early mortality. In some instances, abnormalities associated with SPG49 are similar to those seen in hereditary sensory and autonomic neuropathy (HSAN) which is a group of conditions that affect sensory and autonomic neurons. As such, SPG49 is thought to be related to a specific HSAN called HSAN9.

Further linking SPG49 and HSAN9 is mutations in the tectonin-beta propeller repeat containing 2 (TECPR2) gene. TECPR2 is a multi-domain protein comprised of three WD repeats at the N-terminus. TECPR2 further comprises an LC3-interacting region at its C-terminus. An unstructured region spans the middle of the TECPR2 protein thereby separating the N- and C-terminal domains. Both SPG49 and HSAN9 exhibit an autosomal recessive inheritance pattern. Mutation of TECPR2 is also associated with a hereditary form of spastic paraparesis which is characterized by progressive spasticity and paralysis of the legs. Additionally, TECPR2 mutation is linked to birdshot chorioretinopathy which is characterized by inflammation of the choroid and retina.

TECPR2 is known in the art by several aliases including KIAA0329, WD repeat-containing protein KIAA0329/KIAA0297, KIAA0297, HSAN9, and SPG49. The TECPR2 gene is found at chromosomal location 14q32.31. TECPR2 comprises 20 exons. Alternative splicing of the TECPR2 gene gives rise to different protein isoforms including TECPR2 isoform 1 and TECPR2 isoform 2. A nucleic acid comprising a TECPR2 sequence may comprise mRNAs (or the corresponding DNA sequence) of a TECPR2 sequence known in the art, such as NM_001172631.3 or NM_014844.5, NCBI mRNA sequences including, but not limited to, AB002295.1 AB002327.1 AK127721.1, AK296395.1, AK299690.1, BC030791.1, BC136647.1, BC142667.1, BC142715.1, BP365224.1, or BQ23338.1, or Ensembl IDs including ENST00000557786, ENST00000558678, ENST00000559124, ENST00000560060, ENST00000561099, or ENST00000561228. An amino acid sequence (e.g., one encoded by a nucleic acid comprising a TECPR2 sequence) comprising TECPR2 may include, but is not limited to, NCBI protein sequences NP_001166102.1 (which comprises sequences corresponding to the NM_001172631.1 mRNA sequence) or NP_055659.2 (which comprises sequences corresponding to the NM_014844.5 mRNA sequence).

In some embodiments, a TECPR2-associated disease or disorder occurs as a result of one or more substitutions, deletions, and/or insertions in the nucleotide sequence of a naturally-occurring version of a human TECPR2 sequence corresponding to a nucleic acid sequence found at chromosomal location 14q32.31 (see, e.g., NCBI RefSeq NC_000014.9). In some embodiments, a TECPR2-associated disease or disorder occurs due to mutation of a regulatory sequence that naturally effects TECPR2 expression (e.g., the TECPR2 promoter). In some embodiments, a TECPR2-associated disease or disorder occurs as a result of one or more substitutions, deletions, or insertions in the amino acid sequence corresponding to the protein product encoded by the TECPR2 gene sequence found at chromosomal location 14q32.31 (see, e.g., NCBI RefSeq NC_000014.9), such as the protein product provided in the amino acid sequence set forth as follows:

(SEQ ID NO: 6)
MASISEPVTFREFCPLYYLLNAIPTKIQKGFRSIVVYLTALDTNGDYIAV
GSSIGMLYLYCRHLNQMRKYNFEGKTESITVVKLLSCFDDLVAAGTASGR
VAVFQLVSSLPGRNKQLRRFDVTGIHKNSITALAWSPNGMKLFSGDDKGK
IVYSSLDLDQGLCNSQLVLEEPSSIVQLDYSQKVLLVSTLQRSLLFYTEE
KSVRQIGTQPRKSTGKFGACFIPGLCKQSDLTLYASRPGLRLWKADVHGT
VQATFILKDAFAGGVKPFELHPRLESPNSGSCSLPERHLGLVSCFFQEGW
VLSWNEYSIYLLDTVNQATVAGLEGSGDIVSVSCTENEIFFLKGDRNIIR
ISSRPEGLTSTVRDGLEMSGCSERVHVQQAEKLPGATVSETRLRGSSMAS
SVASEPRSRSSSLNSTDSGSGLLPPGLQATPELGKGSQPLSQRFNAISSE
DFDQELVVKPIKVKRKKKKKKTEGGSRSTCHSSLESTPCSEFPGDSPQSL
NTDLLSMTSSVLGSSVDQLSAESPDQESSFNGEVNGVPQENTDPETFNVL
EVSGSMPDSLAEEDDIRTEMPHCHHAHGRELLNGAREDVGGSDVTGLGDE
PCPADDGPNSTQLPFQEQDSSPGAHDGEDIQPIGPQSTFCEVPLLNSLTV
PSSLSWAPSAEQWLPGTRADEGSPVEPSQEQDILTSMEASGHLSTNLWHA
VTDDDTGQKEIPISERVLGSVGGQLTPVSALAASTHKPWLEQPPRDQTLT
SSDEEDIYAHGLPSSSSETSVTELGPSCSQQDLSRLGAEDAGLLKPDQFA
ESWMGYSGPGYGILSLVVSEKYIWCLDYKGGLFCSALPGAGLRWQKFEDA
VQQVAVSPSGALLWKIEQKSNRAFACGKVTIKGKRHWYEALPQAVFVALS
DDTAWIIRTSGDLYLQTGLSVDRPCARAVKVDCPYPLSQITARNNVVWAL
TEQRALLYREGVSSFCPEGEQWKCDIVSERQALEPVCITLGDQQTLWALD
IHGNLWFRTGIISKKPQGDDDHWWQVSITDYVVFDQCSLFQTIIHATHSV
ATAAQAPVEKVADKLRMAFWSQQLQCQPSLLGVNNSGVWISSGKNEFHVA
KGSLIGTYWNHVVPRGTASATKWAFVLASAAPTKEGSFLWLCQSSKDLCS
VSAQSAQSRPSTVQLPPEAEMRAYAACQDALWALDSLGQVFIRTLSKSCP
TGMHWTRLDLSQLGAVKLTSLACGNQHIWACDSRGGVYFRVGTQPLNPSL
MLPAWIMIEPPVQPAGVSLVSVHSSPNDQMLWVLDSRWNVHVRTGITEEM
PVGTAWEHVPGLQACQLALSTRTVWARCPNGDLARRYGVTDKNPAGDYWK
KIPGSVSCFTVTASDELWAVGPPGYLLQRLTKTFSHSHGTQKSSQAAMPH
PEDLEDEWEVI

In some embodiments, expression of a protein comprising the amino acid sequence of SEQ ID NO: 6 to a cell, or cell population, (e.g., such as those in a subject with SPG49 or HSAN9) is therapeutic for a TECPR2-associated disease or disorder. In some embodiments, expression of a TECPR2 protein (e.g., one comprising the sequence of SEQ ID NO: 6) is achieved by delivering a nucleic acid to a cell, or cell population, that encodes a TECPR2 protein, such as any one of SEQ ID NOs: 1-5, 7, 9, 11, 13, 15, 7, 18, 19, or 27. In some embodiments, the nucleic acid comprises a naturally-occurring TECPR2 sequence (e.g., a coding sequence). In some embodiments, the nucleic acid may comprise a codon-optimized TECPR2 sequence, such as the nucleic acid set forth as follows (or one having the same amino acid coding sequence but having a different stop codon):

(SEQ ID NO: 17)
ATGGCGAGCATCTCAGAGCCGGTCACGTTTAGAGAGTTCTGTCCACTTTA
TTACCTTCTTAATGCAATTCCAACTAAAATCCAGAAAGGATTTCGATCTA
TAGTAGTTTATTTGACCGCATTGGATACGAACGGCGACTACATCGCGGTT
GGCTCAAGCATCGGAATGCTCTATTTGTACTGTCGGCACCTCAATCAGAT
GAGGAAATATAACTTCGAAGGCAAGACCGAATCCATTACAGTTGTGAAGC
TCCTCTCCTGTTTCGACGACCTGGTGGCCGCCGGCACCGCCTCCGGCCGA
GTTGCAGTGTTCCAACTTGTATCAAGCCTCCCCGGCAGGAACAAGCAACT
GAGAAGATTTGACGTTACGGGTATTCATAAGAATTCCATAACGGCTCTGG
CATGGAGCCCGAACGGGATGAAACTGTTCAGCGGTGACGATAAAGGGAAG
ATCGTGTACTCTTCTCTGGATCTGGACCAAGGGCTTTGCAACTCCCAACT
TGTTCTCGAGGAACCATCTTCTATCGTCCAGCTTGACTACAGTCAGAAGG
TCTTGCTTGTGTCAACCCTTCAGCGAAGCCTTCTTTTTTATACGGAAGAG
AAGTCCGTTCGGCAGATTGGGACGCAACCCCGCAAAAGCACCGGTAAGTT
TGGCGCATGTTTTATCCCAGGACTGTGTAAACAATCTGACTTGACACTTT
ATGCCTCACGCCCAGGTCTGAGACTCTGGAAAGCCGACGTACACGGCACT
GTACAAGCTACATTTATCTTGAAGGACGCTTTCGCCGGCGGCGTTAAGCC
CTTCGAACTGCATCCCAGATTGGAGTCCCCCAATAGCGGTTCCTGTTCCC
TTCCAGAGAGGCATCTGGGTCTTGTTTCCTGTTTCTTTCAGGAAGGCTGG
GTGCTCAGCTGGAACGAATACTCAATATACCTGCTTGATACTGTGAACCA
AGCTACTGTTGCGGGGCTGGAGGGAAGCGGTGATATTGTCAGCGTGTCAT
GCACTGAAAATGAGATTTTTTTTCTCAAAGGGGATCGAAATATAATTCGG
ATCTCATCCCGGCCCGAAGGACTTACTAGCACTGTAAGAGACGGACTGGA
GATGAGCGGATGTTCCGAACGCGTGCACGTACAACAGGCTGAGAAGCTGC
CCGGAGCTACTGTGAGTGAAACACGGTTGCGGGGCTCATCCATGGCTTCT
TCAGTTGCAAGCGAACCGCGATCACGGTCATCATCATTGAACTCAACTGA
TAGCGGCAGTGGACTGCTTCCCCCGGGATTGCAGGCTACCCCCGAACTCG
GTAAAGGTTCTCAACCTCTCTCTCAGCGCTTCAACGCGATCTCCTCAGAA
GATTTCGACCAGGAGTTGGTTGTTAAGCCCATCAAAGTCAAGCGCAAAAA
GAAAAAAAAAAAGACCGAAGGGGGCTCCCGGAGTACGTGCCACTCCTCTC
TTGAGAGTACTCCGTGTAGTGAGTTTCCAGGCGACAGTCCGCAGTCTCTG
AACACAGACCTTTTGAGTATGACAAGCTCAGTGCTTGGGAGCAGTGTAGA
CCAACTTTCAGCTGAGTCACCGGATCAGGAGTCTTCTTTCAATGGCGAGG
TTAATGGTGTACCACAGGAGAATACAGATCCCGAAACATTCAACGTCCTC
GAAGTCTCTGGTAGTATGCCAGATTCCCTCGCTGAAGAAGATGACATACG
GACGGAAATGCCCCATTGCCACCACGCGCATGGTCGAGAGCTTCTTAACG
GAGCAAGGGAAGACGTAGGGGGCTCCGACGTAACGGGGTTGGGTGATGAA
CCGTGCCCGGCTGACGACGGTCCCAATAGTACACAgTTGCCGTTCCAAGA
ACAGGACAGTTCCCCCGGCGCGCATGATGGAGAGGACATCCAGCCGATAG
GTCCACAAAGTACTTTCTGCGAGGTTCCACTTCTCAACTCACTCACTGTC
CCGAGCTCTCTTTCATGGGCCCCGTCAGCAGAACAATGGTTGCCAGGAAC
GCGCGCTGATGAAGGCTCCCCGGTTGAACCATCCCAAGAGCAGGACATAC
TGACATCCATGGAAGCTAGTGGGCACCTTTCTACTAATCTGTGGCACGCG
GTCACGGATGACGATACGGGTCAGAAGGAGATACCAATCTCCGAGCGCGT
ACTCGGCTCCGTAGGCGGACAACTTACTCCCGTATCTGCGCTCGCAGCCA
GTACCCACAAGCCATGGCTCGAGCAGCCACCTCGAGATCAAACTCTGACG
TCATCAGATGAAGAGGACATTTACGCCCACGGGCTGCCCTCTTCTTCCTC
AGAGACATCCGTTACAGAGTTGGGTCCGTCTTGCTCACAACAAGATTTGT
CAAGACTTGGTGCCGAAGATGCTGGTCTCCTGAAACCTGATCAGTTTGCA
GAGTCCTGGATGGGCTACTCTGGCCCGGGCTATGGGATACTCTCCCTGGT
CGTCTCCGAAAAGTATATTTGGTGTCTTGATTATAAGGGGGGTCTCTTCT
GTTCTGCATTGCCGGGAGCAGGCTTGAGATGGCAAAAATTCGAGGATGCA
GTACAACAAGTAGCCGTATCACCTAGTGGAGCATTGCTGTGGAAGATTGA
GCAAAAATCAAATCGGGCGTTTGCTTGTGGAAAGGTAACGATTAAAGGTA
AGCGACACTGGTACGAAGCACTTCCCCAGGCCGTATTTGTTGCGCTGTCC
GATGACACCGCCTGGATCATTCGAACATCTGGGGATTTGTATCTTCAAAC
GGGGCTCAGCGTAGATAGACCTTGTGCTCGGGCGGTAAAAGTGGACTGCC
CATACCCCTTGTCACAGATTACTGCACGAAACAACGTCGTTTGGGCTCTG
ACGGAGCAACGAGCTCTGCTCTATAGGGAAGGTGTAAGCTCTTTCTGCCC
AGAAGGGGAACAGTGGAAGTGTGATATCGTATCAGAGCGCCAAGCTCTCG
AACCCGTATGTATCACACTGGGTGATCAGCAAACGCTTTGGGCTCTTGAC
ATACATGGAAATCTTTGGTTTCGCACAGGCATCATTTCAAAAAAACCACA
GGGAGACGACGACCACTGGTGGCAAGTTTCTATCACGGATTATGTCGTAT
TCGATCAGTGTTCACTTTTCCAGACCATAATCCATGCCACGCATTCCGTG
GCAACCGCGGCTCAAGCTCCCGTAGAAAAGGTGGCAGATAAACTCCGCAT
GGCATTTTGGTCACAACAGCTTCAGTGCCAACCTTCTTTGCTTGGTGTGA
ATAACTCTGGAGTTTGGATTTCCAGTGGTAAAAACGAATTCCACGTTGCT
AAGGGCAGCCTTATTGGTACTTATTGGAATCATGTTGTCCCTAGAGGAAC
AGCCTCTGCGACCAAGTGGGCCTTCGTACTTGCATCAGCGGCGCCAACTA
AGGAGGGTTCATTCCTCTGGCTTTGCCAGTCTAGTAAAGACCTCTGTAGT
GTGTCCGCACAAAGCGCCCAAAGCCGACCAAGCACCGTGCAGCTGCCGCC
GGAGGCCGAGATGAGGGCGTACGCCGCATGTCAAGACGCTTTGTGGGCGC
TTGATTCTCTGGGTCAGGTATTTATTAGGACTCTTTCAAAGAGCTGCCCC
ACGGGCATGCACTGGACGCGGCTGGATCTCTCTCAGCTTGGGGCCGTAAA
GCTCACTTCTCTTGCGTGTGGCAACCAGCATATCTGGGCATGCGATTCCA
GAGGGGGAGTATACTTTCGAGTGGGCACACAACCTCTTAATCCGTCACTG
ATGCTTCCAGCGTGGATAATGATCGAGCCGCCGGTCCAACCCGCGGGTGT
TTCACTTGTTAGTGTTCATTCATCTCCGAATGACCAAATGCTCTGGGTCC
TCGACTCTAGATGGAATGTTCACGTACGGACGGGTATTACAGAGGAGATG
CCTGTAGGTACGGCTTGGGAGCATGTCCCTGGCCTGCAGGCATGTCAACT
GGCTCTGTCCACTAGGACGGTCTGGGCCAGGTGTCCAAATGGGGACCTTG
CCAGACGGTACGGAGTGACAGACAAGAATCCTGCAGGTGACTATTGGAAG
AAAATACCCGGTAGTGTAAGCTGTTTCACCGTCACGGCCTCAGATGAACT
GTGGGCTGTGGGACCACCTGGTTACCTCCTCCAGCGCTTGACTAAGACAT
TCTCCCATTCCCACGGCACCCAGAAAAGTTCACAAGCTGCTATGCCACAT
CCAGAAGACCTGGAAGATGAGTGGGAAGTTATTTGA

In other embodiments, a TECPR2 sequence comprises the sequence set forth in SEQ ID NO: 17 but differs with respect to its stop codon (e.g., the 3′-most TGA of SEQ ID NO: 17 is replaced with a TAA codon or a TAG codon). However, in some embodiments, the nucleic acid may comprise a codon-optimized TECPR2 sequence as set forth in SEQ ID NO: 27.

TECPR2 protein is highly expressed in nervous system tissues where it plays a role in the autophagy pathway by regulating the formation of autophagosomes. Impaired autophagy results from mutation of TECPR2 resulting in altered function of axons and dendrites. Fibroblast cell line models and mouse models of TECPR2 mutation exhibit accumulation of autophagosomes indicating that TECPR2 facilitates targeting of autophagosomes to lysosomes. Additionally, TECPR2 also exhibits high expression in the testes.

TECPR2 binding interactions are well known in the art and include, but are not necessarily limited to, chemokine (C-C motif) receptor 2 (CCR2), DEAD box polypeptide 58 (DDX58), DnaJ (Hsp40) homolog, subfamily B, member 5 (DNAJB5), GABA(A) receptor-associated protein (GABARAP), heterogenous nuclear ribonucleoprotein L (HNRNPL), heat shock 70 kDa protein 8 (HSPA8), NudC domain containing 3 (NUDCD3), protein phosphatase, Mg²⁺/Mn²⁺ dependent 1A (PPM1A), RAS oncogene family member RAB5A, vacuolar protein sorting 16 homolog (VPS16), vacuolar protein sorting 18 homolog (VPS18), vacuolar protein sorting 33 homolog A (VPS33A), vacuolar protein sorting 41 homolog (VPS41), and YTH domain containing 1 (YTHDC1).

In some embodiments, a TECPR2-associated disease or disorder may occur as a result of a mutation that alters the function of a protein involved in a pathway related to TECPR2 (e.g., the autophagy pathway), a pathway that alters TECPR2 function (e.g., a degradation pathway that increases TECPR2 degradation), or a protein that interacts with TECPR2, such the ones described herein.

### Nucleic Acids

In some embodiments, the present disclosure provides nucleic acids for expression of a gene or a gene variant. In some embodiments, nucleic acids comprise a coding sequence of a gene or a gene variant. In some embodiments, nucleic acids comprise a sequence which, when mutated, is implicated in a TECPR2-associated disease or disorder (e.g., a TECPR2 sequence, such as a human TECPR2 sequence or coding sequence thereof). In some embodiments, nucleic acids described herein are useful in treating a TECPR2-associated disease or disorder (e.g., SPG49 and HSAN9).

In some embodiments, nucleic acids comprise a TECPR2 sequence (e.g., a human TECPR2 coding sequence encoding a human TECPR2 protein) or a codon-optimized TECPR2 sequence (e.g., a codon-optimized human TECPR2 sequence encoding a human TECPR2 protein). In some embodiments, nucleic acids comprise a codon-optimized TECPR2 sequence, such as the sequences set forth in SEQ ID NO: 17 or 27. In some embodiments, nucleic acids comprise a TECPR2 sequence that is not the nucleic acid sequence set forth in SEQ ID NO: 17 or 27, yet it encodes a TECPR2 protein comprising the amino acid sequence of SEQ ID NO: 6. In some embodiments, a nucleic acid comprises a transgene (e.g., a transgene encoding a TECPR2 sequence or a codon-optimized variant thereof). In some embodiments, a nucleic acid comprises an expression cassette (e.g., one comprising a TECPR2 sequence operably linked to one or more regulatory elements). In some embodiments, a nucleic acid is a vector (e.g., a recombinant adeno-associated viral vector). In some embodiments, a nucleic acid comprises one or more of the sequences set forth in SEQ ID NOs: 1-5 or 7-27. In some embodiments, a nucleic acid further comprises an additional sequence described herein (e.g., one encoding an RNA that does not correspond to a TECPR2 sequence, such as an interfering RNA, mRNA encoding a Cas nuclease, and/or a guide RNA, etc.).

In some embodiments, nucleic acids of the present disclosure comprise approximately 1-10,000 nucleotides. In some embodiments, nucleic acids of the present disclosure comprise approximately 1-10, 10-20, 20-30, 30-40, 40-50, 50-60, 60-70, 70-80, 80-90, 90-100, 100-200, 200-300, 300-400, 400-500, 500-600, 600-700, 700-800, 800-900, 900-1,000, 1,000-1,100, 1,100-1,200, 1,200-1,300, 1,300-1,400, 1,400-1,500, 1,500-1,600, 1,600-1,700, 1,700-1,800, 1,800-1,900, 1,900-2,000, 2,000-2,100, 2,100-2,200, 2,200-2,300, 2,300-2,400, 2,400-2,500, 2,500-2,600, 2,600-2,700, 2,700-2,800, 2,800-2,900, 2,900-3,000, 3,000-3,100, 3,100-3,200, 3,200-3,300, 3,300-3,400, 3,400-3,500, 3,500-3,600, 3,600-3,700, 3,700-3,800, 3,900-4,000, 4,000-4,100, 4,100-4,200, 4,200-4,300, 4,300-4,400, 4,400-4,500, 4,500-4,600, 4,600-4,700, 4,700-4,800, 4,800-4,900, 4,900-5,000, 5,000-5,100, 5,100-5,200, 5,200-5,300, 5,300-5,400, 5,400-5,500, 5,500-5,600, 5,600-5,700, 5,700-5,800, 5,800-5,900, 5,900-6,000, 6,000-6,100, 6,100-6,300, 6,200-6,300, 6,300-6,400, 6,400-6,500, 6,500-6,600, 6,600-6,700, 6,700-6,800, 6,800-6,900, 6,900-7,000, 7,000-7,100, 7,100-7,200, 7,200-7,300, 7,300-7,400, 7,400-7,500, 7,500-7,600, 7,600-7,700, 7,700-7,800, 7,800-7,900, 7,900-8,000, 8,000-8,100, 8,100-8,200, 8,200-8,300, 8,300-8,400, 8,400-8,500, 8,500-8,600, 8,600-8,700, 8,700-8,800, 8,800-8,900, 8,900-9,000, 9,000-9,100, 9,100-9,200, 9,200-9,300, 9,300-9,400, 9,400-9,500, 9,500-9,600, 9,600-9,700, 9,700-9,800, 9,800-9,900, or 9,900-10,000 nucleotides. However, such embodiments should not be considered limiting as, in other embodiments, nucleic acids of the present disclosure may comprise more than 10,000 nucleotides (e.g., approximately 15,000, 20,000, 25,000, 30,000 or more nucleotides).

In some embodiments, a nucleic acid sequence comprises DNA. In some embodiments, a nucleic acid sequence comprises RNA. In some embodiments, a nucleic acid sequence comprises a combination of DNA and RNA. In some embodiments, a nucleic acid comprises double-stranded DNA. In some embodiments, a nucleic acid comprises single-stranded DNA. In some embodiments, a nucleic acid comprises double-stranded RNA. In some embodiments, a nucleic acid comprises single-stranded RNA. In some embodiments, a nucleic acid comprises a double-stranded nucleic acid comprising a combination of DNA and RNA nucleotides. In some embodiments, a nucleic acid comprises a double-stranded nucleic acid (e.g., DNA, RNA, or a combination of DNA/RNA) comprising gaps (e.g., stretches of sequence about, 1, 5, 10, 25, 50, 100 or more nucleotides in length) of single-stranded sequence at any position within the molecule. In some embodiments, a nucleic acid is linear. In some embodiments, a nucleic acid is circular. In some embodiments, a nucleic acid comprises at least one single-stranded nick.

In some embodiments, a nucleic acid comprises naturally-occurring and/or a non-naturally occurring nucleobases (bases). In some embodiments, a nucleic acid comprises known base analogues of DNA and RNA, such as, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

Accordingly, it should be recognized that a nucleic acid comprising a TECPR2 sequence described herein (e.g., a codon-optimized human TECPR2 coding sequence, such as one comprising the sequence set forth in SEQ ID NO: 17 or 27), which comprises a substitution, addition, and/or deletion of at least one nucleotide, is further provided by the present disclosure. In some embodiments, such a nucleic acid may comprise a plurality (e.g., 2, 3, 4, 5, 6, 7, 8, 9, 10, 10-20, 20-30, 30-40, 40-50, 50-100, 100-250, 250-500, 500-1000, etc.) of nucleotide positions which differ relative to any one of SEQ ID NOs: 1-5 or 7-27 are further provided by the present disclosure. In other embodiments, such a nucleic acid may comprise at least 75% or more (e.g., 80-85%, 85-90%, 90-95%, 95%, 96%, 97%, 98%, or 99%) sequence identity to any one of SEQ ID NOs: 1-5 or 7-27. In some embodiments, such a nucleic acid may comprise a transgene, expression cassette, and/or vector. In some embodiments, such a nucleic acid may be used to treat a TECPR2-associated disease or disorder.

In some embodiments, a nucleic acid comprises a substitution and/or is absent of one or more sequences encoded by SEQ ID NOs: 1-5, 7-16, or 18-26, yet still comprises a TECPR2 sequence of SEQ ID NO: 17 or 27. In some embodiments, such a nucleic acid may comprise one or more of an enhancer, promoter, polyA signal, or splice site (e.g., addition a splice site that effects splicing of TECPR2) that is not found in any one of SEQ ID NOs: 1-5 or 7-27. In some embodiments, such a nucleic acid may comprise a TECPR2 sequence described herein (e.g., one comprising a sequence that is at least approximately 85%, 90%, 95%, or 99% identical to SEQ ID NO: 17 or 27, or one that comprises the sequence of SEQ ID NOs: 17 or 27) or a transgene or expression cassette thereof which has been moved into a new vector.

For example, in some embodiments, a nucleic acid may comprise one or more of the MECP2 promoter, chimeric intron, TECPR2 coding sequence, or SV40 polyA signal of SEQ ID NO: 1 but differ with regards to the sequences between nucleotide positions 1-169 and/or 4973-9056 of SEQ ID NO: 1. In some embodiments, a nucleic acid may comprise one or more of the JeT promoter, TECPR2 coding sequence, or SV40 polyA signal of SEQ ID NO: 2 but differ with regards to the sequences between nucleotide positions 1-169 and/or 4976-8818 of SEQ ID NO: 2. In some embodiments, a nucleic acid may comprise one or more of the human synapsin promoter, MVM intron, TECPR2 coding sequence, or SV40 polyA signal of SEQ ID NO: 3 but differ with regards to the sequences between nucleotide positions 1-169 and/or 5095-9177 of SEQ ID NO: 3. In some embodiments, a nucleic acid may comprise one or more the miniCMV promoter, MVM intron, TECPR2 coding sequence, or SV40 polyA signal of SEQ ID NO: 4 but differ with regards to the sequences between nucleotide positions 1-169 and/or 4852-8934 of SEQ ID NO: 4. In some embodiments, a nucleic acid may comprise one or more the minTK promoter, MVM intron, TECPR2 coding sequence, or SV40 polyA signal of SEQ ID NO: 5 but differ with regards to the sequences between nucleotide positions 1-169 and/or 4756-8837 of SEQ ID NO: 5. In some embodiments, a nucleic acid may comprise one or more the mU1a promoter, Kozak sequence, TECPR2 coding sequence, or Proudfoot polyA signal of SEQ ID NO: 18 but differ with regards to the sequences between nucleotide positions 1-145 and/or 4751-8807 of SEQ ID NO: 18. In some embodiments, a nucleic acid may comprise inverted terminal repeat sequences which are different from those provided in SEQ ID NOs: 1-5 and 18. In some embodiments, a nucleic acid may comprise any or all of the sequences in SEQ ID NOs: 1-5, 7, 9, 11, 13, 15, 18, or 19 but may differ in a stop codon that is operably linked to the TECPR2 coding sequence to control translation of the encoded protein.

Transgenes

In some embodiments, a transgene is used to express a TECPR2 sequence. In some embodiments, a transgene may be employed to correct, reduce, eliminate, or otherwise ameliorate gene deficiencies. In some embodiments, such gene deficiencies may include deficiencies in which normal genes are expressed at less than normal levels, are expressed at normal or near-normal levels but having a gene product with abnormal activity, or deficiencies in which the functional gene product is not expressed. In some embodiments, the transgene sequence encodes a therapeutic protein or polypeptide which is to be expressed in a target cell. Some embodiments of the present disclosure also include using multiple transgenes.

TECPR2 is a regulator of the autophagy pathway in nervous system cells where it regulates targeting of autophagosomes to lysosomes. Mutations in or perturbations in the function of TECPR2 are associated with SPG49 and HSAN9.

In some embodiments of the disclosed rAAV vectors, the transgene is TECPR2, such as human TECPR2. In some embodiments, the transgene is a TECPR2 sequence that has been codon-optimized for expression in a mammalian cell. In some embodiments, the transgene is a TECPR2 sequence that has been codon-optimized for expression in human cells. In some embodiments, the transgene comprises a sequence encoded by nucleotide sequences set forth in any one of SEQ ID NOs: 1-5 or 7-27.

Regulatory Elements

In some embodiments, a nucleic acid (e.g., a rAAV vector) comprises one or more regions comprising a sequence that facilitates expression of a TECPR2 sequence (e.g., a nucleic acid comprising inverted terminal repeats which flank a heterologous nucleic acid comprising the TECPR2 sequence). In some embodiments, a TECPR2 sequence is operably linked to one or more regulatory sequences (e.g., a heterologous nucleic acid flanked by inverted terminal repeats may comprise one or more regulatory sequences operably linked to a TECPR2 sequence).

In some embodiments, a promoter drives transcription of the nucleic acid sequence that it regulates, thus, it is typically located at or near the transcriptional start site of a gene. In some embodiments, a promoter may have, e.g., a length of 100 to 1000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (“drive”) transcriptional initiation and/or expression of) that sequence. Numerous such sequences are known in the art.

Promoters that may be used in accordance with the present disclosure may comprise any promoter that can drive the expression of the transgenes in the of the subject. In some embodiments, the promoter may be a tissue-specific promoter. A “tissue-specific promoter”, as used herein, refers to promoters that can only function in a specific type of tissue, e.g., cells of the nervous system. Thus, a “tissue-specific promoter” is not able to drive the expression of the transgenes in other types of tissues. In some embodiments, the promoter that may be used in accordance with the present disclosure is a neuron-promoter. Non-limiting examples of tissue-specific promoters and/or regulatory elements that may be used in any of the disclosed nucleic acids include (1) desmin, creatine kinase, myogenin, alpha myosin heavy chain, and natriuretic peptide, specific for muscle cells, and (2) albumin, alpha-1-antitrypsin, hepatitis B virus core protein promoters, specific for liver cells. In some embodiments, the promoter is a muscle creatine kinase promoter, such as muscle-specific promoter MHCK9.

Examples of neuron-specific promoters and other control elements are known in the art. Non-limiting examples of neuron-specific promoters include neuron-specific enolase promoter, an aromatic amino acid decarboxylase promoter, a neurofilament promoter, a synapsin promoter, a thy-1 promoter, a serotonin receptor promoter, a GnRH promoter, an L7 promoter, a DNMT promoter, an enkephalin promoter, a myelin basic protein promoter, a CMV enhancer/platelet-derived growth factor-O promoter, a motor neuron-specific gene Hb9 promoter, and an alpha subunit of Ca(²⁺)-calmodulin-dependent protein kinase II promoter. In some embodiments, the promoter is a mammalian TECPR2 promoter. In some embodiments, the promoter is rat TECPR2. In some embodiments, the promoter is human TECPR2. In some embodiments, the promoter is mouse MECP2. In some embodiments, the promoter is JeT. In some embodiments, the promoter is human synapsin. In some embodiments, the promoter is a minimal core promoter of the thymidine kinase gene (minTK) (see, e.g., Jiang et al. (2008). Regulation of the MAD1 promoter by G-CSF. Nuc Acids Res. 36, (5): 1517-1531 which is incorporated by reference in its entirety herein). In some embodiments, the promoter is a minimal cytomegalovirus (minCMV) promoter (see, e.g., Ede et al. (2016). Quantitative Analyses of Core Promoters Enable Precise Engineering of Regulated Gene Expression in Mammalian Cells. ACS Synth Biol. 5 (5): 395-404 which is incorporated by reference in its entirety herein). In treating TECPR2-associated diseases or disorders as provided for herein, promoters are advantageous at least due to the reduced possibility of off-target expression of the transgene(s), thereby effectively increasing the delivered dose to the nervous system tissues and enhancing therapy. Non-limiting examples of expression regulatory sequences include promoters, insulators, silencers, response elements, introns (e.g., minimal minute virus (MVMi) introns), enhancers, initiation sites, termination signals, and polyA signals (e.g., sequences that promote addition of polyA tails to RNA transcripts). Any combination of such regulatory sequences is contemplated herein (e.g., a promoter and an enhancer).

In some embodiments, the promoter is mouse MECP2. In some embodiments, the promoter is JeT. In some embodiments, the promoter is human synapsin. In some embodiments, the promoter is a modified human synapsin. In some embodiments, the promoter is minCMV. In some embodiments, the promoter is minTK. In some embodiments, the promoter is mU1a. In some embodiments, the promoter comprises the nucleic acid sequence of any one of SEQ ID NOs: 8, 10, 12, 14, 16, or 20).

In some embodiments, to achieve appropriate expression levels of the protein of interest (TECPR2), any of a number of promoters suitable for use in the selected target cell may be employed. In some embodiments, the promoter may be, e.g., a constitutive promoter, tissue-specific promoter, inducible promoter, or a synthetic promoter. For example, in some embodiments, constitutive promoters of different strengths can be used. In some embodiments, an rAAV vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of non-viral constitutive promoters include various housekeeping gene promoters, as exemplified by the β-actin promoter, including the chicken β-actin promoter (CBA).

In some embodiments, inducible promoters and/or regulatory elements may also be contemplated for achieving appropriate expression levels of the protein or polypeptide of interest. Non-limiting examples of suitable inducible promoters include those from genes, such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Synthetic promoters are also contemplated herein. In some embodiments, a synthetic promoter may comprise, e.g., regions of known promoters, regulatory elements, transcription factor binding sites, enhancer elements, repressor elements, and the like.

Described herein are non-limiting examples of promoters that are encoded by rAAV vectors of the present disclosure. Accordingly, in some embodiments, the promoter may have a sequence having identity to SEQ ID NOs: 8, 10, 12, 14, 16, or 20. The promoters of the disclosure may comprise nucleotide sequences that have at least 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to the sequences set forth as SEQ ID NOs: 8, 10, 12, 14, 16, or 20. In several embodiments, the promoter has 100% identity to the sequences set forth as SEQ ID NOs: 8, 10, 12, 14, 16, or 20.

In some embodiments, enhancer elements can function in combination with other regulatory elements to increase the expression of a transgene. In some embodiments, the enhancer elements are upstream (positioned 5′) of the transgene. Non-limiting embodiments of enhancer elements include nucleotide sequences comprising, e.g., a 100 base pair element from Simian virus 40 (SV40 late 2XUSE), a 35 base pair element from Human Immunodeficiency Virus 1 (HIV-1 USE), a 39 base pair element from ground squirrel hepatitis virus (GHV USE), a 21 base pair element from adenovirus (Adenovirus L3 USE), a 21 base pair element from human prothrombin (hTHGB USE), a 53 base pair element from human C2 complement gene (hC2 USE), truncations of any of the foregoing, and any combinations of the foregoing.

Non-limiting polyadenylation (polyA) signals include nucleotide sequences comprising, e.g., a 624 base pair polyadenylation signal from human growth hormone (hGH), a 135 base pair polyadenylation signal from simian virus 40 (sV40 late), a 49 base pair synthetic polyadenylation signal from rabbit beta-globin (SPA), a 250 base pair polyadenylation signal from bovine growth hormone (bGH), truncations of any of the foregoing, and combinations of the foregoing.

In some embodiments of the disclosed rAAV vectors, the two or more transgenes are operably controlled by a single promoter. In some embodiments, each of the two or more transgenes are operably controlled by a distinct promoter.

In some embodiments, the rAAV vectors of the present disclosure further comprise an Internal Ribosome Entry Site (IRES). An IRES is a nucleotide sequence that allows for translation initiation in the middle of a messenger RNA (mRNA) sequence as part of the greater process of protein synthesis. Usually, in eukaryotes, translation can be initiated only at the 5′ end of the mRNA molecule, since 5′ cap recognition is required for the assembly of the initiation complex. In some embodiments, the IRES is located between the transgenes.

In such embodiments, the proteins encoded by different transgenes are translated individually (e.g., versus translated as a fusion protein). In some embodiments, the rAAV vectors of the present disclosure comprise at least, in order from 5′ to 3′, a first adeno-associated virus (AAV) inverted terminal repeat (ITR) sequence, a promoter operably linked to a first transgene, an IRES operably linked to a second transgene, a polyadenylation signal, and a second AAV inverted terminal repeat (ITR) sequence. In some embodiments, the rAAV vectors of the present disclosure comprise in order from 5′ to 3′, a first adeno-associated virus (AAV) inverted terminal repeat (ITR) sequence, a promoter operably linked to a TECPR2 transgene, an IRES operably linked to a second transgene, a polyadenylation signal, and a second AAV inverted terminal repeat (ITR) sequence. In some embodiments, the rAAV vectors of the present disclosure comprise in order from 5′ to 3′, a first adeno-associated virus (AAV) inverted terminal repeat (ITR) sequence, a promoter operably linked to a codon-optimized human TECPR2 transgene, an IRES operably linked to a second transgene, a polyadenylation signal, and a second AAV inverted terminal repeat (ITR) sequence.

In some embodiments, the rAAV vectors of the present disclosure comprise a chimeric intron sequence. The chimeric intron may contain a restriction enzyme (endonuclease) cleavage site.

Expression Cassettes

In some embodiments, expression cassettes comprise, at a minimum, one or more regulatory sequences operably linked to another nucleic acid sequence (e.g., a TECPR2 sequence) for the purposes of expression. In some embodiments, an expression cassette comprises a transgene (e.g., one comprising a TECPR2 sequence) and its regulatory sequences. In some embodiments, where the cassette is designed to be expressed from an rAAV, the expression cassette is flanked on each side by 5′ and 3′ AAV ITRs. Accordingly, in some embodiments, a nucleic acid which is heterologous to ITRs that flank the heterologous nucleic acid may comprise an expression cassette described herein. In some embodiments, the ITR's may be full-length, or one or both of the ITRs may be truncated. In some embodiments, the rAAV is pseudotyped, i.e., the AAV capsid is from a different source AAV than that the AAV which provides the ITRs. In some embodiments, the ITRs of AAV serotype 2 are used. In additional embodiments, the ITRs of AAV serotype 1 are used. However, ITRs from other suitable sources may be selected.

In some embodiments, the expression cassette comprises a TECPR2 sequence and one or more operably linked regulatory elements. In some embodiments, the expression cassette comprises a human TECPR2 gene. In some embodiments, the expression cassette comprises a TECPR2 transgene and associated regulatory sequences. In some embodiments, the expression cassette further comprises an additional nucleic acid sequence that modulates endogenous TECPR2 gene expression. In some embodiments, the nucleic acid sequence that modulates endogenous TECPR2 gene expression encodes an interfering RNA (e.g., shRNAs, siRNAs, miRNAs, ncRNAs, piRNAs, pro-siRNAs, etc.), exon-skipping RNA, gapmer, enzymatic RNA, guide RNA (gRNA) (e.g., sgRNAs) of a CRISPR/Cas editing system (e.g., Cas9-based genome editing and derivatives thereof, such as base editing and prime editing). In some embodiments, the expression cassette comprises a TECPR2 transgene and associated regulatory sequences but does not include a region modulating endogenous TECPR2 gene expression. In some embodiments, a nucleic acid comprising the expression cassette with the functional TECPR2 transgene is administered (e.g., to a subject). In some embodiments, the expression of the functional TECPR2 transgene is sufficient to provide therapeutic benefits to a subject. In some embodiments, the expression of the functional TECPR2 transgene provides a gain of function to a subject.

In some embodiments, described herein are non-limiting examples of expression cassettes that are encoded by rAAV vectors of the present disclosure. Accordingly, in some embodiments, the expression cassette may have a sequence having identity to SEQ ID NOs: 7, 9, 11, 13, 15, 17, 19, or 27. In some embodiments, the expression cassette may comprise nucleotide sequences that have at least 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to the sequences set forth as SEQ ID NOs: 7, 9, 11, 13, 15, 17, 19, or 27. In some embodiments, the expression cassette has 100% identity to the sequences set forth as SEQ ID NOs: 7, 9, 11, 13, 15, 17, 19, or 27. In some embodiments, described herein are non-limiting examples of TECPR2 nucleotide sequences that are encoded by rAAV vectors of the present disclosure. Accordingly, in some embodiments, the TECPR2 sequence may have a sequence having identity to SEQ ID NO: 17 or 27. The TECPR2 sequence of the disclosure may comprise nucleotide sequences that have at least 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to the sequences set forth as SEQ ID NO: 17 or 27. In several embodiments, the TECPR2 sequence has 100% identity to the sequences set forth as SEQ ID NO: 17 or 27.

rAAV Particles

In further embodiments, provided herein are rAAV viral particles and compositions comprising such rAAV particles. In some embodiments, rAAV particles comprise a viral capsid comprising at least one AAV capsid protein and one or more transgenes as described herein, which is encapsidated by the viral capsid. Methods of producing rAAV particles are known in the art and are commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, in some embodiments, a plasmid containing rAAV vector sequences may be combined with one or more helper and/or packaging nucleic acids (e.g., plasmids), such as those comprising, e.g., an AAV rep gene (e.g., encoding Rep78, Rep68, Rep52 and Rep40) and an AAV cap gene (encoding one or more of VP1, VP2, and VP3, including a modified VP3 region as described herein), and transfected into a producer cell line such that the rAAV particle can be packaged and subsequently purified.

In some embodiments, rAAV particles or particles within an rAAV preparation disclosed herein, may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV an rAAV particle refers to the serotype of the capsid proteins of the recombinant virus. In some embodiments, the rAAV particle is rAAV6 or rAAV9. In some embodiments, the rAAV particle is AAVrh.74. In a preferred embodiment, the rAAV particle is AAVrh74. In an additional preferred embodiment, the rAAV is AAV9. In several embodiments, an rh74 AAV is mutated to advantageously enhance delivery to nervous system tissue, for example by a tryptophan to arginine mutation at amino acid 505 of VP1 capsid, and/or other mutations, as described in PCT Publication WO 2019/178412, which is incorporated in its entirety by reference herein. Non-limiting examples of derivatives, pseudotypes, and/or other vector types include, but are not limited to, AAVrh.10, AAVrh.74, AAV2/1, AAV2/5, AAV2/6, AAV2/8, AAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShHIO, AAV2 (Y->F), AAV8 (Y733F), AAV2.15, AAV2.4, AAVM41, and AAVr3.45.

Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan Al, Schaffer D V, Samulski R J). In particular embodiments, the capsid of any of the herein disclosed rAAV particles is of the AAVrh.10 serotype. In a preferred embodiment, the capsid of the rAAV particle is AAVrh10 serotype. In some embodiments, the capsid is of the AAV2/6 serotype. In some embodiments, the rAAV particle is a pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al, J. Virol., 75:7662-7671, 2001; Halbert et al, J. Virol., 74:1524-1532, 2000; Zolotukhin et al, Methods, 28:158-167, 2002; and Auricchio et al., Hum. Molec. Genet., 10:3075-3081, 2001).

In some embodiments, rAAV genomes comprise nucleic acids (e.g., transgenes, expression cassettes, and/or vectors) described herein. In some embodiments, rAAV genomes (e.g., an rAAV vector) of the present disclosure comprise at least, in order from 5′ to 3′, a first adeno-associated virus (AAV) inverted terminal repeat (ITR) sequence, a heterologous nucleic acid, and a second AAV inverted terminal repeat (ITR) sequence. In some embodiments, the heterologous nucleic acid comprises a TECPR2 sequence (e.g., a transgene or an expression cassette thereof) operably linked to one or more regulatory elements. In some embodiments, rAAV vectors of the present disclosure comprise at least, in order from 5′ to 3′, a first adeno-associated virus (AAV) inverted terminal repeat (ITR) sequence, a promoter operably linked to a transgene, a polyadenylation signal, and a second AAV inverted terminal repeat (ITR) sequence.

In some embodiments, an rAAV genome is circular. In some embodiments, an rAAV genome is linear. In some embodiments, an rAAV genome is single-stranded. In some embodiments, an rAAV genome is double-stranded. In some embodiments, an rAAV genome is a self-complementary rAAV vector.

In some embodiments, described herein are non-limiting examples of constructs that can be used to produce rAAV vectors. The constructs illustrated below comprise the linearized plasmid sequences set forth as SEQ ID NOs: 1-5 and 18. Accordingly, in some embodiments, the rAAV vector may have a sequence having identity to SEQ ID NOs: 1-5 and 18. In some embodiments, the vectors of the disclosure may comprise nucleotide sequences that have at least 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to the sequences set forth as SEQ ID NOs: 1-5 and 18. In some embodiments, the rAAV has 100% identity to the sequences set forth as SEQ ID NOs: 1-5 and 18. In some embodiments, any of the disclosed rAAV vectors have at least 85% sequence identity to any of the disclosed sequence groupings arranged in sequence, without any gaps between the subject sequences. In some embodiments, any of the disclosed rAAV vectors have at least 85% sequence identity to any of the disclosed sequence groupings arranged in sequence, without any gaps between the subject sequences.

In some embodiments, a therapeutic rAAV genome (e.g., encapsidated within an AAV particle) has a sequence i) comprising a transgene, expression construct, or rAAV genome of any one of SEQ ID NOs: 1-5 or 7-27, or ii) having at least 85% identity to a transgene, expression construct, or rAAV genome of any one of SEQ ID NOs: 7, 9, 11, 13, 15, 17, 19, or 27. Accordingly, in some embodiments, a therapeutic rAAV vector has a sequence comprising one or more coding sequences, promoters, other regulatory elements, and/or ITRs of any of SEQ ID NOs: 1-5, 7, 9, 11, 13, 15, 17, 18, 19, or 27.

rAAV Vectors and Therapeutic Uses Thereof

In some embodiments, a nucleic acid is a recombinant adeno-associated virus (rAAV) vector. In some embodiments, a nucleic acid is a single-stranded or self-complementary rAAV nucleic acid vector. In some embodiments, an rAAV particle is of serotype AAV9. In some embodiments, the rAAV particle is of AAV serotype rh74. In some embodiments, an rAAV particle is of AAV serotype rh10. In some embodiments, a composition comprising a plurality of rAAV particles is provided. In some embodiments, a plurality of rAAV particles may further comprise a pharmaceutically acceptable carrier. In some embodiments, a nucleic acid is pTR2-MCS-Dual. In some embodiments, a nucleic acid comprises a polyA signal (or polyA tail sequence), such as a minimal polyA. Exemplary minimal polyA signals include bovine growth hormone (bGH) and SV40 polyA.

Many serotypes of AAV have been cloned and sequenced. Serotypes 1 and 6 share >99% amino acid homology in their capsid proteins. Of the first six AAV serotypes, serotype 2 is widely characterized and therefore often used in gene transfer studies, however according to embodiments disclosed herein, other AAV serotypes are also used, such as AAV9, AAV20, AAVrh74, AAVrh10, and the like. In some embodiments, repeat administration of a given serotype that would be expected to elicit a humoral immune response is performed in connection with an immune management regimen. In some embodiments, an immune management regimen comprises administration of one or more agents that provide B-cell depletion or ablation, alone, or in conjunction with one or more agents that inhibit one or more aspects of the mTOR pathway. In some embodiments, an anti-CD20 antibody (e.g., rituximab) and rapamycin is administered. In some embodiments, this allows for the repeat administration of a given serotype rAAV with reduced, limited or no immune response to a subsequent dosing of the rAAV. Further information about immune management can found in United States Patent Publication No. US 2017/0049887, published Feb. 23, 2017, the entire contents of which is incorporated by reference herein.

In some embodiments, the therapeutic rAAV vectors, therapeutic rAAV particles, or the composition comprising the therapeutic rAAV particles of the present disclosure, may be used for gene therapy for TECPR2-associated diseases or disorders in a human subject in need thereof, such as SPG49 and/or HSAN9 as provided for herein. In some embodiments, therapeutic rAAV vectors, particles, and compositions comprising the therapeutic rAAV particles may be used for treatment of intellectual disability, spastic ataxic gait, and autonomic dysfunction associated with a TECPR2-associated disease or disorder (e.g.) when administered to a subject in need thereof, e.g., via vascular delivery or injection into cerebrospinal fluid. In some embodiments, therapeutic rAAV vectors, particles, and compositions comprising the rAAV particles drive the concurrent expression of TECPR2 in the of the subject.

In some embodiments, an amino acid sequence which is therapeutic for a TECPR2-associated disease or disorder and encoded by the TECPR2 sequence (e.g., transgene) has at least about 85%, at least 90%, at least 95%, at least 96%, at least 97%, at least 98% or at least 99% (e.g., 100%) sequence identity to the amino acid sequence set forth as SEQ ID NO: 6.

In some embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, or other types of modifications.

In accordance with some embodiments described herein, any of the sequences may be used, or a truncated or mutated form of any of the sequences disclosed herein (and/or included in the accompanying sequence listing) may be used and in any combination.

In some embodiments, the promoter driving expression of the therapeutic nucleic acid can be, but is not limited to, a constitutive promoter, an inducible promoter, a tissue-specific promoter, a neuronal-specific promoter, a muscle-specific promoter, or a synthetic promoter. In some embodiments, the promoter is a neuronal-specific promoter or a muscle-specific promoter. A constitutive promoter can be, but is not limited to, a Herpes Simplex virus (HSV) promoter, a thymidine kinase (TK) promoter, a Rous Sarcoma Virus (RSV) promoter, a Simian Virus 40 (SV40) promoter, a Mouse Mammary Tumor Virus (MMTV) promoter, an Adenovirus E1A promoter, a cytomegalovirus (CMV) promoter, a mammalian housekeeping gene promoter, or a β-actin promoter. In some embodiments, an inducible promoter can be, but is not limited to, a cytochrome P450 gene promoter, a heat shock protein gene promoter, a metallothionein gene promoter, a hormone-inducible gene promoter, an estrogen gene promoter, or a tetVP16 promoter that is responsive to tetracycline. In some embodiments, a muscle-specific promoter can be, but is not limited to, desmin promoter, a creatine kinase promoter (e.g., MHCK9), a myogenin promoter, an alpha myosin heavy chain promoter, or a natriuretic peptide promoter.

In some embodiments, the therapeutic rAAV particle comprises a neural-specific promoter.

In some embodiments, a therapeutic rAAV particle can be serotype 1, serotype 2, serotype 3, serotype 4, serotype 5, serotype 6, serotype 7, serotype 8, serotype 9, serotype 10, serotype 11, serotype 12, serotype rh10, or serotype rh74. In some embodiments, a therapeutic rAAV particle can also be a pseudo-typed rAAV particle.

In some embodiments, the therapeutic rAAV particle comprises a nucleic acid with a sequence having at least 85% sequence identity to a transgene, expression construct, or rAAV genome of any one of SEQ ID NOs: 1-5, 7, 9, 11, 13, 15, 17, 18, 19, or 27.

Pharmaceutical Formulations and Administration

In some embodiments, a composition (e.g., a pharmaceutical composition useful in treating a TECPR2-associated disease or disorder) may comprise one or more of the nucleic acids of the present disclosure. In some embodiments, the nucleic acid comprises a transgene, expression cassette, and/or vector described herein. In some embodiments, a composition (e.g., a pharmaceutical composition useful in treating a TECPR2-associated disease or disorder) may comprise one or more of the rAAV particles described herein. In some embodiments, said compositions (e.g., pharmaceutical compositions useful in treating a TECPR2-associated disease or disorder) may comprise one or more nucleic acids having at least 85% identity to any one of SEQ ID NOs: 5-7 or 7-27. In some embodiments, said compositions (e.g., pharmaceutical compositions useful in treating a TECPR2-associated disease or disorder) may comprise one or more rAAV particles comprising a recombinant genome comprising a nucleic acid having at least 85% identity to any one of SEQ ID NOs: 5-7 or 7-27. In some embodiments, compositions described herein comprise nucleic acids having the sequence of any one of SEQ ID NOs: 5-7 or 7-27 (e.g., the sequence of any one of SEQ ID NOs: 1-5, 7, 9, 11, 13, 15, 17 18, 19, or 27).

In some embodiments, compositions described herein may further comprise a pharmaceutical excipient, buffer, or diluent, and may be formulated for administration to target cell ex vivo or in situ in an animal, and particularly a human being. In some embodiments, such compositions may further comprise a liposome, a lipid, a lipid complex, a microsphere, a microparticle, a nanosphere, or a nanoparticle, or may be otherwise formulated for administration to the cells, tissues, organs, or body of a subject in need thereof. In some embodiments, such compositions may be formulated for use in a variety of therapies, such as for example, in the amelioration, prevention, and/or treatment of conditions, such as peptide deficiency, polypeptide deficiency, peptide overexpression, polypeptide overexpression, including for example, conditions which result in diseases or disorders as described herein.

Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

In some embodiments, these formulations may contain at least about 0.1% of the therapeutic agent (e.g., therapeutic rAAV particle or preparation) or more, although the percentage of the active ingredient(s) may, of course, be varied and may conveniently be between about 1 or 2% and about 70% or 90% or more of the weight or volume of the total formulation. Naturally, the amount of therapeutic agent(s) in each therapeutically-useful composition may be prepared in such a way that a suitable dosage will be obtained in any given unit dose of the compound. In some embodiments, factors, such as solubility, bioavailability, biological half-life, route of administration, product shelf life, as well as other pharmacological considerations will be contemplated by one skilled in the art when preparing such pharmaceutical formulations. Additionally, a variety of dosages and treatment regimens may be desirable.

In some circumstances, it will be desirable to deliver the therapeutic rAAV particles or preparations in suitably formulated pharmaceutical compositions disclosed herein; either subcutaneously, intraocularly, intravitreally, parenterally, subcutaneously, intravenously, intracerebro-ventricularly, intramuscularly, intrathecally, orally, intraperitoneally, or by oral or nasal inhalation, or by direct injection to one or more cells, tissues, or organs. In some embodiments, the therapeutic rAAV particles or the composition comprising the therapeutic rAAV particles of the present invention are delivered systemically via intravenous injection, particularly in those for treating a human. In some embodiments, the therapeutic rAAV particles or the composition comprising the therapeutic rAAV particles of the present invention are injected directly into the cerebrospinal fluid of the subject. In some embodiments, direct injection to human nervous system tissue is preferred, for example, if delivery is performed concurrently with a surgical procedure or interventional procedure whereby access to the nervous system tissue is improved.

In some embodiments, pharmaceutical formulations of the compositions suitable for injectable use include sterile aqueous solutions or dispersions. In some embodiments, the formulation is sterile and fluid to the extent that easy syringability exists. In some embodiments, the form is stable under the conditions of manufacture and storage, and is preserved against the contaminating action of microorganisms, such as bacteria and fungi. In some embodiments, the carrier may be a solvent or dispersion medium containing, for example, water, saline, ethanol, polyol (e.g., glycerol, propylene glycol, and liquid polyethylene glycol, and the like), suitable mixtures thereof, vegetable oils or other pharmaceutically acceptable carriers, such as those that are Generally Recognized as Safe (GRAS) by the United States Food and Drug Administration. In some embodiments, proper fluidity may be maintained, for example, by the use of a coating, such as lecithin, by the maintenance of the required particle size in the case of dispersion and by the use of surfactants. In fact, there is virtually no limit to other components that may also be included, as long as the additional agents do not cause a significant adverse effect upon contact with the target cell (e.g., a cell of the nervous system) or host tissues. In some embodiments, the therapeutic rAAV particles or preparations may thus be delivered along with various other pharmaceutically acceptable agents as required in the particular instance. Such compositions may be purified from host cells or other biological sources, or alternatively may be chemically synthesized as described herein.

In some embodiments, the amount of therapeutic rAAV particle or preparation, and/or therapeutic rAAV vector compositions and time of administration of such compositions will be within the purview of the skilled artisan having benefit of the present teachings. In some embodiments, however, the administration of therapeutically-effective amounts of the compositions of the present disclosure may be achieved by a single administration, such as for example, a single injection of sufficient numbers of infectious particles to provide therapeutic benefit to the patient undergoing such treatment. In some embodiments, it may be desirable to provide multiple or successive administrations of the rAAV particle or preparation, and/or rAAV vector compositions, either over a relatively short, or a relatively prolonged period of time, as may be determined by the medical practitioner overseeing the administration of such compositions.

In some embodiments, toxicity and efficacy of the compositions utilized in methods of the present invention may be determined by standard pharmaceutical procedures, using either cells in culture or experimental animals to determine the LD₅₀ (the dose lethal to 50% of the population). In some embodiments, the dose ratio between toxicity and efficacy is the therapeutic index and it may be expressed as the ratio LD₅₀/ED₅₀. In some embodiments, those compositions that exhibit large therapeutic indices are preferred. In some embodiments, while compositions that exhibit toxic side effects may be used, care should be taken to design a delivery system that minimizes the potential damage of such side effects. In preferable embodiments, the dosage of compositions as described herein lies generally within a range that includes an ED₅₀ with little or no toxicity. In some embodiments, the dosage may vary within this range depending upon the dosage form employed and the route of administration utilized.

Other aspects of the present disclosure relate to methods and preparations for use with a subject, such as human or non-human subjects, a target cell in situ in a subject, or a target cell derived from a subject. In some embodiments, the subject is a mammal. In some embodiments, the subject is a companion animal. “A companion animal”, as used herein, refers to pets and other domestic animals. Non-limiting examples of companion animals include dogs and cats; livestock, such as horses, cattle, pigs, sheep, goats, and chickens; and other animals, such as mice, rats, guinea pigs, and hamsters. In some embodiments, the subject is a human subject.

In some embodiments, one or more pharmaceutically acceptable excipients (including vehicles, carriers, diluents, and/or delivery polymers) are added to the pharmaceutical compositions including a therapeutic, thereby forming a pharmaceutical formulation suitable for in vivo delivery to a subject, such as a human.

In some embodiments, a pharmaceutical composition or medicament includes a pharmacologically effective amount of at least one of the therapeutic and optionally one or more pharmaceutically acceptable excipients. In some embodiments, pharmaceutically acceptable excipients (excipients) are substances other than the Active Pharmaceutical ingredient (API, therapeutic product) that are intentionally included in the drug delivery system. In some embodiments, excipients do not exert or are not intended to exert a therapeutic effect at the intended dosage. In some embodiments, excipients may act to a) aid in processing of the drug delivery system during manufacture, b) protect, support, or enhance stability, bioavailability or patient acceptability of the API, c) assist in product identification, and/or d) enhance any other attribute of the overall safety, effectiveness, of delivery of the API during storage or use. In some embodiments, a pharmaceutically acceptable excipient may or may not be an inert substance.

In some embodiments, excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

In some embodiments, pharmaceutical compositions can contain other additional components commonly found in pharmaceutical compositions. In some embodiments, such additional components can include, but are not limited to, anti-pruritics, astringents, local anesthetics, or anti-inflammatory agents (e.g., antihistamine, diphenhydramine).

In some embodiments, the carrier can be, but is not limited to, a solvent or dispersion medium containing, for example, water, ethanol, polyol (for example, glycerol, propylene glycol, and liquid polyethylene glycol), and suitable mixtures thereof. In some embodiments, a carrier may also contain adjuvants, such as preservatives, wetting agents, emulsifying agents, and dispersing agents. In some embodiments, a carrier may also contain isotonic agents, such as sugars, polyalcohols, sodium chloride, and the like into the compositions.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, rAAVs or pharmaceutical compositions as described herein, may be formulated for administration to a target cell ex vivo or in situ in an animal, and particularly a human being. The rAAVs or pharmaceutical compositions can be administered by a variety of routes. In some embodiments, administration routes included, but are not limited to, intravenous, intra-arterial, subcutaneous, intramuscular, intrahepatic, intraperitoneal and/or local delivery to a target tissue. In some embodiments, a plurality of injections, or other administration types, are provided, e.g., 2, 3, 4, 5, 6, 7, 8, 9, 10 or more injections. In some embodiments, routes of administration may be combined, if desired. Depending on the embodiment, the first and second rAAV need not be administered the same number of times (e.g., the first rAAV may be administered 1 time, and the second vector may be administered three times). In some embodiments, the dosing is intramuscular administration.

In some embodiments, the number of rAAV particles administered to a subject may be on the order ranging from about 10⁶ to about 10¹⁴ particles or about 10³ to about 10¹³ particles, or any values in between for either range, such as e.g., about 10⁶, 10⁷, 10⁸, 10⁹, 10¹⁰, 10¹¹, 10¹², 10¹³, or 10¹⁴ particles. In some embodiments, the number of rAAV particles administered to a subject may be on the order ranging from about 10⁶ to about 10¹⁴ vector genomes (vgs) or 10³ to 10¹⁵ vgs, or any values in between for either range, such as e.g., about 10⁶, 10⁷, 10⁸, 10⁹, 10¹⁰, 10¹¹, 10¹², 10¹³, or 10¹⁴ vgs. In some embodiments, rAAV particles can be administered as a single dose, or divided into two or more administrations as may be required to achieve therapy of the particular disease, or disorder being treated. In some embodiments, any of said titers of viral particles may be administered to a subject provided in a solution with a volume ranging from about 0.0001 mL to about 10 mLs.

In some embodiments, administration of an injectable aqueous solution, for example, the solution may be suitably buffered, if necessary, and the liquid diluent first rendered isotonic with sufficient saline or glucose. In some embodiments, these particular aqueous solutions are especially suitable for intravenous, intramuscular, intravitreal, subcutaneous and intraperitoneal administration. In some embodiments, in this connection, a sterile aqueous medium that can be employed will be known to those of skill in the art in light of the present disclosure. In some embodiments, for example, one dosage may be dissolved in 1 mL of isotonic NaCl solution and either added to 1000 mL of hypodermoclysis fluid or injected at the proposed site of infusion, (see, for example, “Remington's Pharmaceutical Sciences” 15th Edition, pages 1035-1038 and 1570-1580). In several embodiments, the rAAV formulation will comprise, consist of, or consist essentially of active rAAV ingredient, a mono-basic buffer (e.g., sodium phosphate mono-basic buffer, a di-basic salt (e.g., sodium phosphate di-basic), a sodium-based tonicifier (e.g., sodium chloride tonicifier), a non-sodium tonicifier (e.g., magnesium chloride hexahydrate tonicifier), a surfactant (e.g., poloxamer 188 surfactant), and water. In some embodiments, the rAAV formulation will comprise, consist of, or consist essentially of active rAAV ingredient, sodium phosphate mono-basic buffer, sodium phosphate di-based, sodium chloride tonicifier, magnesium chloride hexahydrate tonicifier, poloxamer 188 surfactant, and water. In some embodiments, the active rAAV ingredient is present in the formulation according to the vector genome amounts provided for herein. In some embodiments, the mono-basic buffer (e.g., sodium phosphate mono-basic buffer) is present in the formulation at a concentration between about 0.2 mg/mL and about 0.5 mg/mL. In some embodiments, the di-basic salt (e.g., sodium phosphate di-basic) is present in the formulation at a concentration between about 1.5 mg/mL and about 4 mg/mL. In some embodiments, the sodium-based tonicifier (e.g., sodium chloride tonicifier) is present in the formulation at a concentration between about 8 mg/mL and about 12 mg/mL. In some embodiments, the non-sodium tonicifier (e.g., magnesium chloride hexahydrate tonicifier) is present in the formulation at a concentration between about 0.1 mg/mL and about 0.35 mg/mL. In some embodiments, the surfactant (e.g., poloxamer 188 surfactant) is present in the formulation at a concentration between about 0.05 mg/mL and about 0.8 mg/mL. In some embodiments, water is present to bring the volume of the formulation (e.g., a dosage unit) to 1 mL.

In some embodiments, some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

In some embodiments, sterile injectable solutions are prepared by incorporating the rAAV particles or preparations in the required amount in the appropriate solvent with several of the other ingredients enumerated above, as required, followed by filtered sterilization. In some embodiments, dispersions are prepared by incorporating the various sterilized active ingredients into a sterile vehicle that contains the basic dispersion medium and the other ingredients from those enumerated above. In some embodiments, in the case of sterile powders for the preparation of sterile injectable solutions, the preferred methods of preparation are vacuum-drying and freeze-drying techniques, which yield a powder of the active ingredient plus any additional desired ingredient from a previously sterile-filtered solution thereof.

In some embodiments, the amount of rAAV particle or preparation and time of administration of such particle or preparation will be within the purview of the skilled artisan having benefit of the present teachings. In some embodiments, however, that the administration of therapeutically-effective amounts of the rAAV particles or preparations of the present disclosure may be achieved by a single administration, such as for example, a single injection of sufficient numbers of infectious particles to provide therapeutic benefit to the patient undergoing such treatment. Alternatively, in some embodiments, it may be desirable to provide multiple or successive administrations of the rAAV particle or preparation, either over a relatively short, or a relatively prolonged period of time, as may be determined by the medical practitioner overseeing the administration of such compositions.

In some embodiments, rAAV particles may be administered in combination with other agents as well, such as e.g., proteins or polypeptides or various pharmaceutically-active agents, including one or more administrations of therapeutic polypeptides, biologically active fragments, or variants thereof. In fact, there is virtually no limit to other components that may also be included, as long as the additional agents do not cause a significant adverse effect upon contact with the target cell (e.g., a cell of the nervous system) or host tissues. In some embodiments, rAAV particles or preparations may thus be delivered along with various other pharmaceutically acceptable agents as required in the particular instance. In some embodiments, such compositions may be purified from host cells or other biological sources, or alternatively may be chemically synthesized as described herein.

In some embodiments, treatment of a subject with a rAAV particles as described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. In some embodiments, the disease, disorder, or symptom is caused by mutation of TECPR2. In some embodiments, the disease or symptom is SPG49 or HSAN9. In some embodiments, the symptom is intellectual disability, spastic ataxic gait, and autonomic dysfunction.

As is apparent to those skilled in the art in view of the teachings of this specification, in some embodiments, an effective amount of viral vector to be added can be empirically determined. In some embodiments, administration can be administered in a single dose, a plurality of doses, continuously or intermittently throughout the course of treatment. Methods of determining the most effective means and dosages of administration are well known to those of skill in the art and will vary with the viral vector, the composition of the therapy, the target cell (e.g., a cell of the nervous system), and the subject being treated. In some embodiments, single and multiple administrations can be carried out with the dose level and pattern being selected by the treating physician.

Kits

Herein are described embodiments wherein compositions including one or more of the disclosed rAAV vectors comprised within a kit for diagnosing, preventing, treating, or ameliorating one or more symptoms of a TECPR2-associated disease or disorder, such as intellectual disability, spastic ataxic gait, and autonomic dysfunction associated with SPG9 and/or HSAN9. In some embodiments, kits may be useful in the diagnosis, prophylaxis, and/or therapy or a human disease, and may be particularly useful in the treatment, prevention, and/or amelioration of one or more symptoms of a TECPR2-disease or disorder, such as intellectual disability, spastic ataxic gait, and autonomic dysfunction associated with SPG9 and/or HSAN9.

In some embodiments, kits comprising one or more of the disclosed rAAV vectors (as well as one or more virions, viral particles, transformed host cells or pharmaceutical compositions comprising such vectors); and instructions for using such kits in one or more therapeutic, diagnostic, and/or prophylactic clinical embodiments are also provided according to several embodiments. In some embodiments, such kits may comprise one or more reagents, restriction enzymes, peptides, therapeutics, pharmaceutical compounds, or means for delivery of the composition(s) to host cells, or to an animal (e.g., syringes, injectables, and the like). In some embodiments, exemplary host cells of the disclosure include HEK293 and C2C12 myoblast cells. In some embodiments, additional exemplary host cells of the disclosure include human cells derived from an induced pluripotent stem cell or a neuronal cell line. In some embodiments, kits include those for treating, preventing, or ameliorating the symptoms of a disease, deficiency, dysfunction, and/or injury, or may include components for the large-scale production of the viral vectors themselves.

In some embodiments, a kit comprises one or more containers or receptacles comprising one or more doses of any of the described therapeutic. In some embodiments, such kits may be therapeutic in nature. In some embodiments, the kit contains a unit dosage, meaning a predetermined amount of a composition comprising, e.g., a described therapeutic with or without one or more additional agents.

In some embodiments, one or more of the components of a kit can be provided in one or more liquid or frozen solvents. In some embodiments, the solvent can be aqueous or non-aqueous. In some embodiments, the formulation in the kit can also be provided as dried powder(s) or in lyophilized form that can be reconstituted upon addition of an appropriate solvent.

In some embodiments, a kit comprises a label, marker, package insert, bar code and/or reader indicating directions of suitable usage of the kit contents. In some embodiments, the kit may comprise a label, marker, package insert, bar code and/or reader indicating that the kit contents may be administered in accordance with a certain dosage or dosing regimen to treat a subject.

In some embodiments, a kit may also contain various reagents, including, but not limited to, wash reagents, elution reagents, and concentration reagents. In some embodiments, such reagents may be readily selected from among the reagents described herein, and from among conventional concentration reagents. As used herein, the term “kit” may be used to describe variations of the portable, self-contained enclosure that includes at least one set of components to conduct one or more of the diagnostic or therapeutic methods of the disclosure.

### EXAMPLES

The following examples are illustrative only and are not intended to be a limitation on the scope of the invention.

Materials and Methods

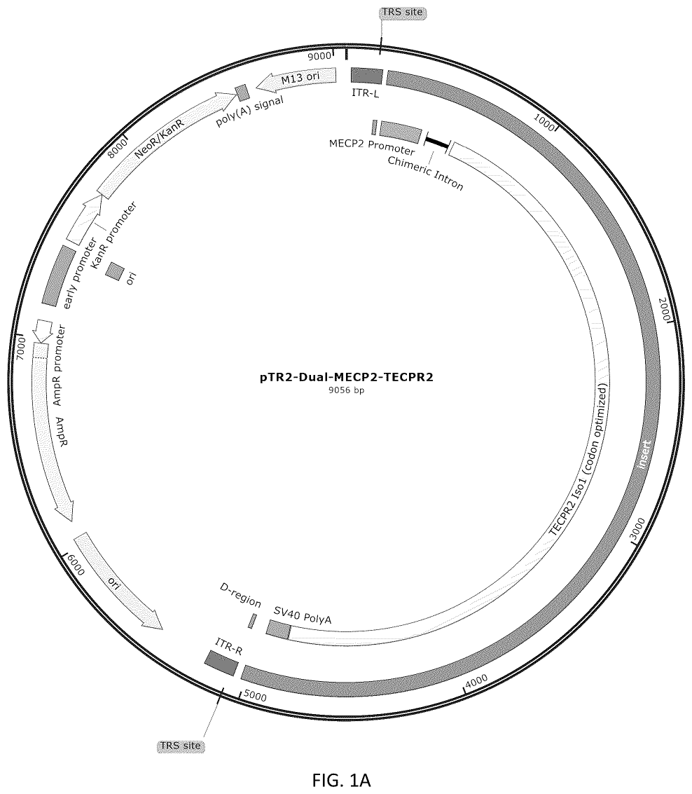

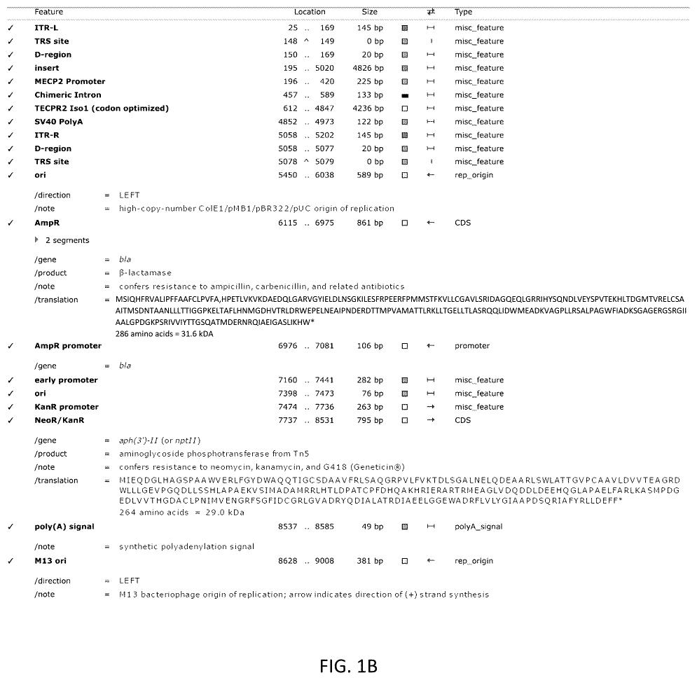

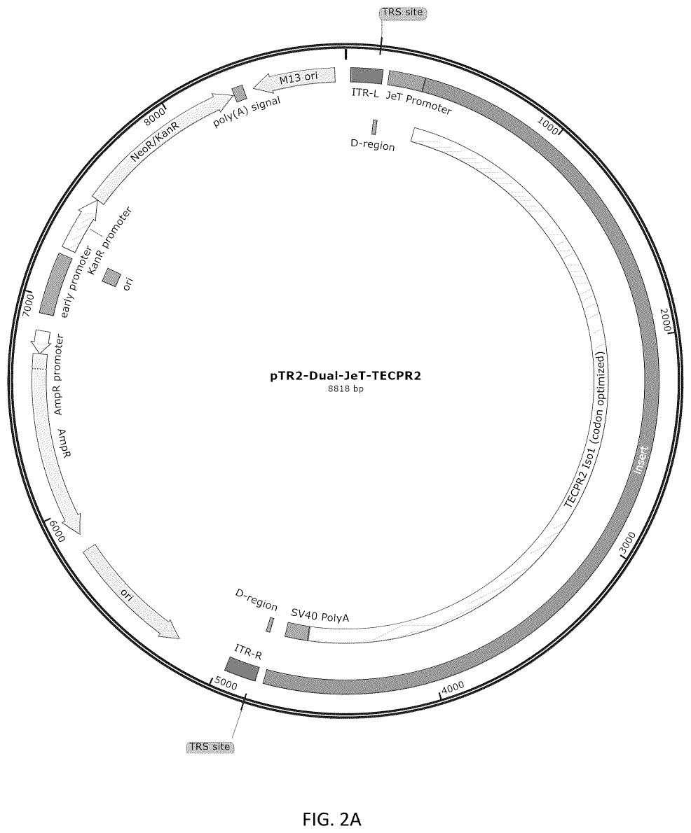

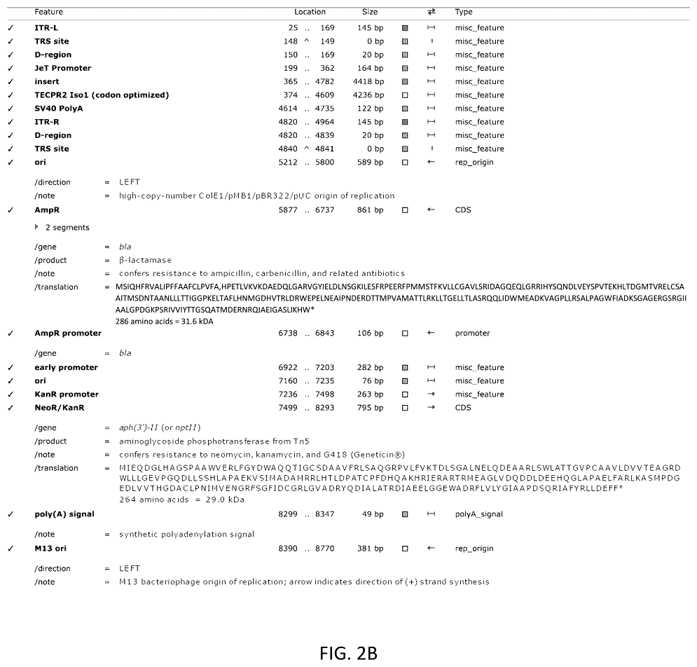

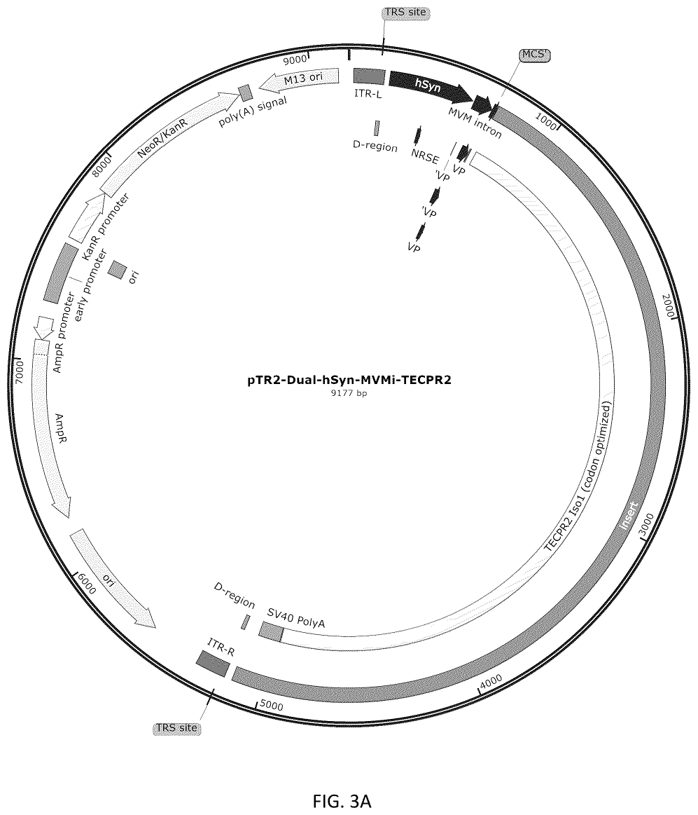

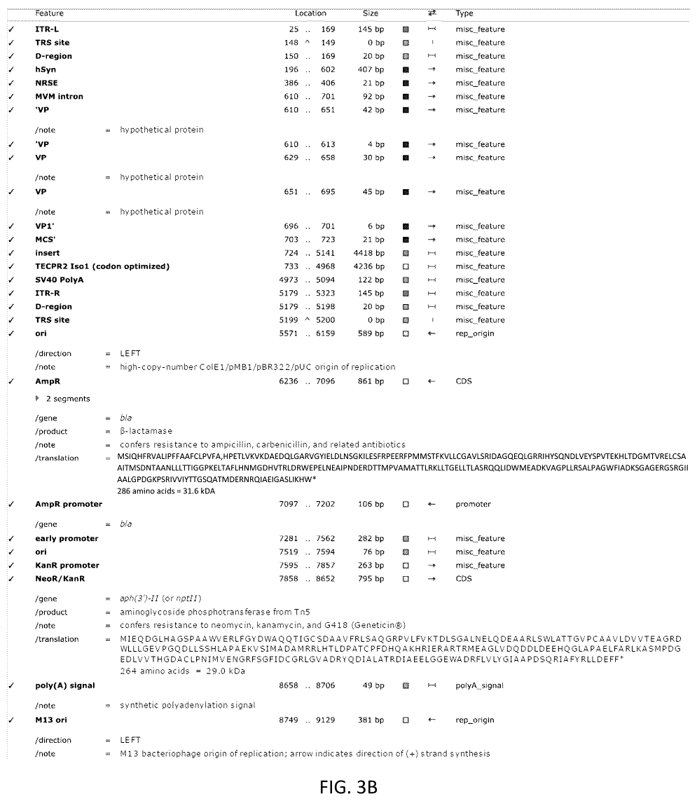

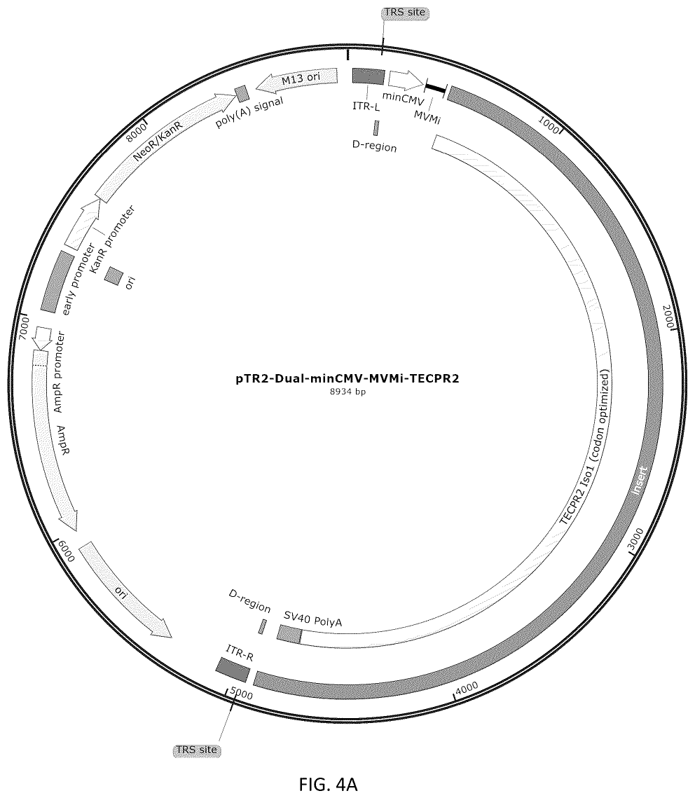

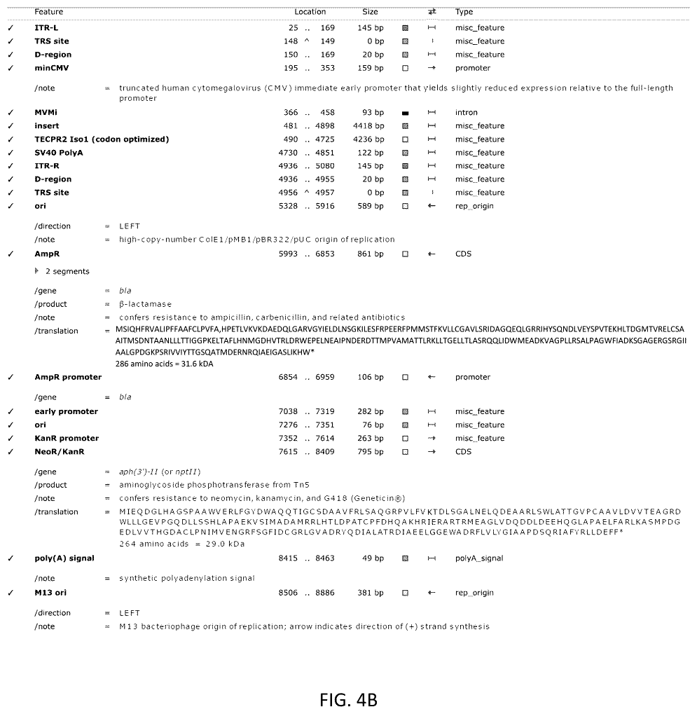

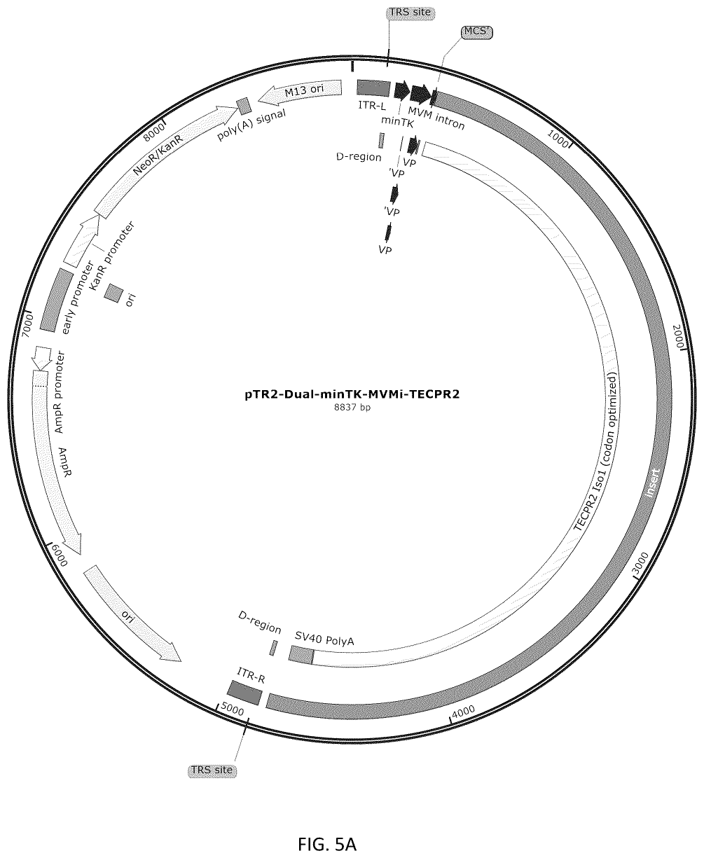

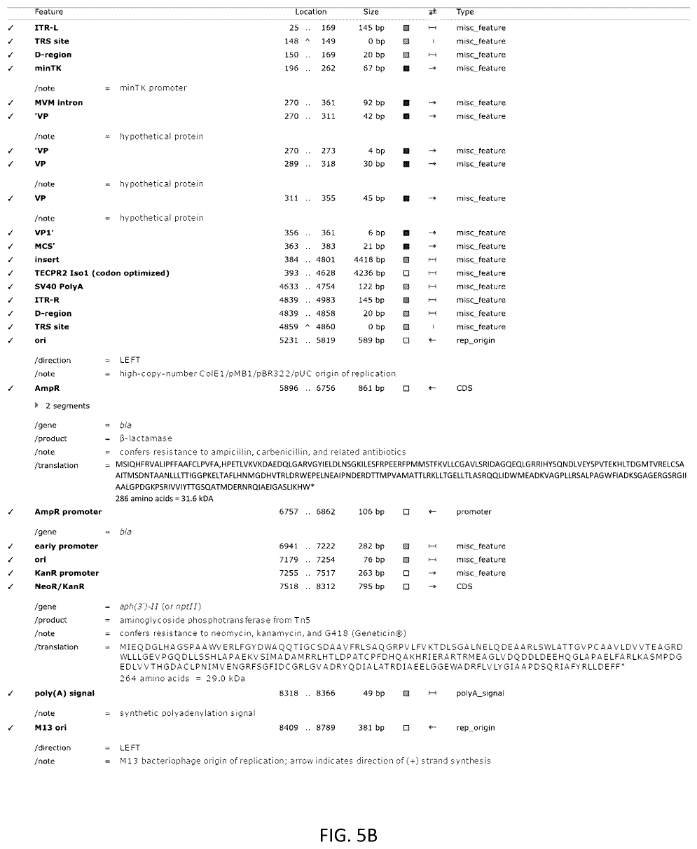

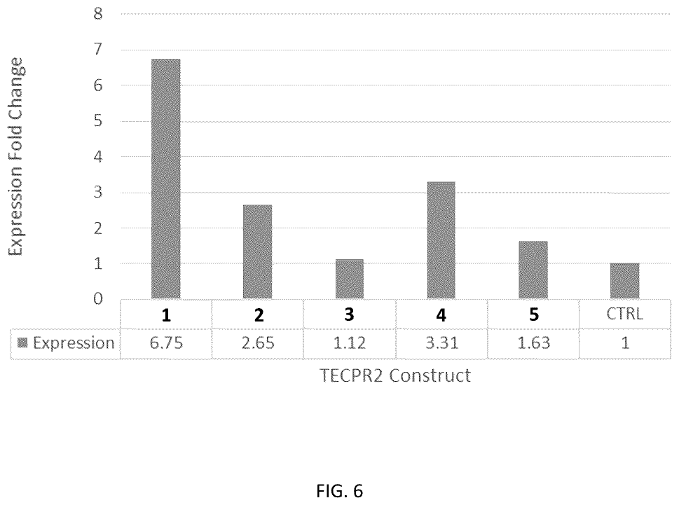

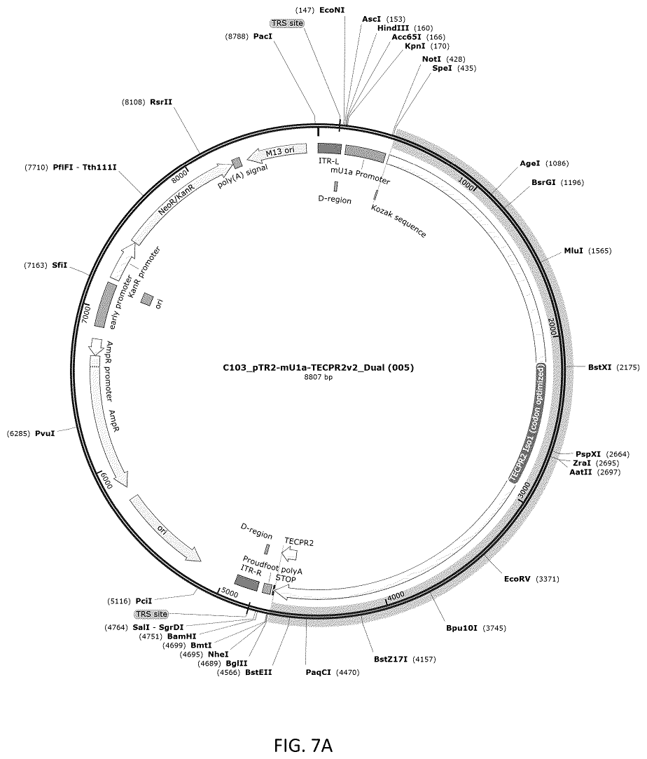

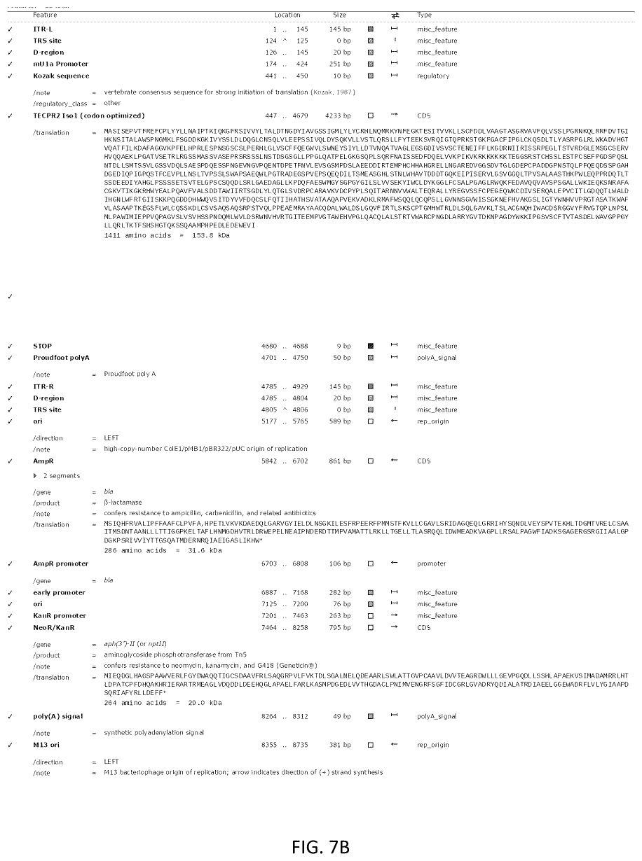

Construct design. Nucleic acids for expressing TECPR2 were engineered, and codon optimized for expression in human tissues. Nucleic acids are subcloned into a plasmid backbone suitable for production of AAV. Nucleic acids comprising single stranded AAV genomes were engineered to comprise the elements as provided in Tables 1-9 below. Schematic representations of the nucleic acids are provided in FIGS. 1-5 and FIG. 7. Tables 1-7 provide sequence information of corresponding nucleic acids in FIGS. 1-5 and FIG. 7. Table 8 shows sequences encoded by said nucleic acids. Table 9 shows promoters encoded by said nucleic acids. Codon optimized human TECPR2 coding sequences encode a protein comprising the amino acid sequence of SEQ ID NO: 6.

AAV production. Recombinant AAV (rAAV) particles comprising each of the Constructs are made by suspension transfection of Expi293F cells with the TECPR2 Constructs and other plasmids needed for rAAV production (e.g., comprising rep and cap sequences) to generate three groups of rAAV comprising AAV9 capsid proteins. Vector is isolated using a capture column followed by an anion exchange column and purified using a cesium chloride gradient to a titer of 2×10¹³ to 5×10¹³ vg/ml.

### Example 1. In Vitro Expression Study

rAAV particles comprising the TECPR2 constructs is made as described above and delivered to HEK293 cells, C2C12 myoblast cells, or cells comprising one or more TECPR2 mutations derived from human induced pluripotent stem cells and/or neuronal cell lines. Quantitative real-time PCR (qPCR) analyses indicated the human TECPR2 (hTECPR2) expression fold change of five gene therapy Constructs (see Tables 2-6 and FIGS. 1-5) following transfection of HEK293T cells, relative to a non-transfected control. The results indicated that all five Constructs are expressed at higher levels (-fold changes of 6.75, 2.65, 1.12, 3.31, and 1.63) than endogenous TECPR2 levels. Further analyses, such as ELISA and/or immunoblot analyses of whole cell lysates are used to validate TECPR2 protein expression.

TABLE 1
Non-Limiting Examples of TECPR2 Therapeutic Nucleic Acids (Constructs 1-6)

| | Length | | | | | | Method of |
|---|---|---|---|---|---|---|---|
| Name | (bp) | Backbone | CDS | Promoter | Intron | polyA | Verification |
| pTR2-Dual- | 9056 | pTR2-MCS-Dual | TECPR2 | Mouse | None | Minimal | Small Digest |
| MECP2- | | | Isoform | MECP2 | | polyA (8) | and |
| RECPR2 | | | 1 (1) | (2) | | | sequencing |
| (Construct 1) | | | | | | | by GeneWiz |
| pTR2-Dual-JeT- | 8818 | pTR2-MCS-Dual | TECPR2 | JeT (3) | None | Minimal | Small Digest |
| TECPR2 | | | Isoform | | | polyA (8) | and |
| (Construct 2) | | | 1 (1) | | | | sequencing |
| | | | | | | | by GeneWiz |
| pTR2-Dual- | 9177 | pTR2-MCS-Dual | TECPR2 | Human | MVMi | Minimal | Small Digest |
| hSyn-MVMi- | | | Isoform | Synapsin | (7) | polyA (8) | and |
| TECPR2 | | | 1 (1) | (4) | | | sequencing |
| (Construct 3) | | | | | | | by GeneWiz |
| pTR2-Dual- | 8934 | pTR2-MCS-Dual | TECPR2 | minCMV | MVMi | Minimal | Small Digest |
| minCMV- | | | Isoform | (5) | (7) | polyA (8) | and |
| MVMi-TECPR2 | | | 1 (1) | | | | sequencing |
| (Construct 4) | | | | | | | by GeneWiz |
| pTR2-Dual- | 8837 | pTR2-MCS-Dual | TECPR2 | minTK (6) | MVMi | Minimal | Small Digest |
| minTK-MVMi- | | | Isoform | | (7) | polyA (8) | and |
| TECPR2 | | | 1 (1) | | | | sequencing |
| (Construct 5) | | | | | | | by GeneWiz |
| C103_pTR2- | 8807 | mU1a- | TECPR2 | mU1a | None | Proudfoot | |
| mU1a- | | TECPR2v2_Dual | Isoform | | | poly(A) | |
| TECPR2v2_Dual | | (002) | 1 (1) | | | | |
| (002) (Construct | | | | | | | |
| 6) | | | | | | | |

TABLE 2
Nucleotide Sequence Information for TECPR2 Construct 1

| SEQ ID NO: | Nucleotide Sequence (5′→3′) |
|---|---|
| 1 | TTAATTAACTGCAGACGCCAGCTGTTGGCCACTCCCTCTCTGCGCGCTCGCTC |
| | GCTCACTGAGGCCGGGCGACCAAAGGTCGCCCGACGCCCGGGCTTTGCCCGG |
| | GCGGCCTCAGTGAGCGAGCGAGCGCGCAGAGAGGGAGTGGCCAACTCCATC |
| | ACTAGGGGTTCCTTCTAGAGGCGCGCCAAGCTTGGTACCCAATTGAGGGCGT |
| | CACCGCTAAGGCTCCGCCCCAGCCTGGGCTCCACAACCAATGAAGGGTAATC |
| | TCGACAAAGAGCAAGGGGTGGGGCGCGGGCGCGCAGGTGCAGCAGCACACA |
| | GGCTGGTCGGGAGGGCGGGGCGCGACGTCTGCCGTGCGGGGTCCCGGCATCG |
| | GTTGCGCGCGCGCTCCCTCCTCTCGGAGAGAGGGCTGTGGTAAAACCCGTCC |
| | GGAAACCACCCCTAGGACCCAAGTCGCAGCCGTTGGATCAGGTAAGTATCAA |
| | GGTTACAAGACAGGTTTAAGGAGACCAATAGAAACTGGGCTTGTCGAGACA |
| | GAGAAGACTCTTGCGTTTCTGATAGGCACCTATTGGTCTTACTGACATCCACT |
| | TTGCCTTTCTCTCCACAGGTGATGATATCGCGGCCGCCGCATGGCGAGCATCT |
| | CAGAGCCGGTCACGTTTAGAGAGTTCTGTCCACTTTATTACCTTCTTAATGCA |
| | ATTCCAACTAAAATCCAGAAAGGATTTCGATCTATAGTAGTTTATTTGACCGC |
| | ATTGGATACGAACGGCGACTACATCGCGGTTGGCTCAAGCATCGGAATGCTC |
| | TATTTGTACTGTCGGCACCTCAATCAGATGAGGAAATATAACTTCGAAGGCA |
| | AGACCGAATCCATTACAGTTGTGAAGCTCCTCTCCTGTTTCGACGACCTGGTG |
| | GCCGCCGGCACCGCCTCCGGCCGAGTTGCAGTGTTCCAACTTGTATCAAGCCT |
| | CCCCGGCAGGAACAAGCAACTGAGAAGATTTGACGTTACGGGTATTCATAAG |
| | AATTCCATAACGGCTCTGGCATGGAGCCCGAACGGGATGAAACTGTTCAGCG |
| | GTGACGATAAAGGGAAGATCGTGTACTCTTCTCTGGATCTGGACCAAGGGCT |
| | TTGCAACTCCCAACTTGTTCTCGAGGAACCATCTTCTATCGTCCAGCTTGACT |
| | ACAGTCAGAAGGTCTTGCTTGTGTCAACCCTTCAGCGAAGCCTTCTTTTTTAT |
| | ACGGAAGAGAAGTCCGTTCGGCAGATTGGGACGCAACCCCGCAAAAGCACC |
| | GGTAAGTTTGGCGCATGTTTTATCCCAGGACTGTGTAAACAATCTGACTTGAC |
| | ACTTTATGCCTCACGCCCAGGTCTGAGACTCTGGAAAGCCGACGTACACGGC |
| | ACTGTACAAGCTACATTTATCTTGAAGGACGCTTTCGCCGGCGGCGTTAAGCC |
| | CTTCGAACTGCATCCCAGATTGGAGTCCCCCAATAGCGGTTCCTGTTCCCTTC |
| | CAGAGAGGCATCTGGGTCTTGTTTCCTGTTTCTTTCAGGAAGGCTGGGTGCTC |
| | AGCTGGAACGAATACTCAATATACCTGCTTGATACTGTGAACCAAGCTACTG |
| | TTGCGGGGCTGGAGGGAAGCGGTGATATTGTCAGCGTGTCATGCACTGAAAA |
| | TGAGATTTTTTTTCTCAAAGGGGATCGAAATATAATTCGGATCTCATCCCGGC |
| | CCGAAGGACTTACTAGCACTGTAAGAGACGGACTGGAGATGAGCGGATGTTC |
| | CGAACGCGTGCACGTACAACAGGCTGAGAAGCTGCCCGGAGCTACTGTGAGT |
| | GAAACACGGTTGCGGGGCTCATCCATGGCTTCTTCAGTTGCAAGCGAACCGC |
| | GATCACGGTCATCATCATTGAACTCAACTGATAGCGGCAGTGGACTGCTTCC |
| | CCCGGGATTGCAGGCTACCCCCGAACTCGGTAAAGGTTCTCAACCTCTCTCTC |
| | AGCGCTTCAACGCGATCTCCTCAGAAGATTTCGACCAGGAGTTGGTTGTTAA |
| | GCCCATCAAAGTCAAGCGCAAAAAGAAAAAAAAAAAGACCGAAGGGGGCTC |
| | CCGGAGTACGTGCCACTCCTCTCTTGAGAGTACTCCGTGTAGTGAGTTTCCAG |
| | GCGACAGTCCGCAGTCTCTGAACACAGACCTTTTGAGTATGACAAGCTCAGT |
| | GCTTGGGAGCAGTGTAGACCAACTTTCAGCTGAGTCACCGGATCAGGAGTCT |
| | TCTTTCAATGGCGAGGTTAATGGTGTACCACAGGAGAATACAGATCCCGAAA |
| | CATTCAACGTCCTCGAAGTCTCTGGTAGTATGCCAGATTCCCTCGCTGAAGAA |
| | GATGACATACGGACGGAAATGCCCCATTGCCACCACGCGCATGGTCGAGAGC |
| | TTCTTAACGGAGCAAGGGAAGACGTAGGGGGCTCCGACGTAACGGGGTTGG |
| | GTGATGAACCGTGCCCGGCTGACGACGGTCCCAATAGTACACAGTTGCCGTT |
| | CCAAGAACAGGACAGTTCCCCCGGCGCGCATGATGGAGAGGACATCCAGCC |
| | GATAGGTCCACAAAGTACTTTCTGCGAGGTTCCACTTCTCAACTCACTCACTG |
| | TCCCGAGCTCTCTTTCATGGGCCCCGTCAGCAGAACAATGGTTGCCAGGAAC |
| | GCGCGCTGATGAAGGCTCCCCGGTTGAACCATCCCAAGAGCAGGACATACTG |
| | ACATCCATGGAAGCTAGTGGGCACCTTTCTACTAATCTGTGGCACGCGGTCA |
| | CGGATGACGATACGGGTCAGAAGGAGATACCAATCTCCGAGCGCGTACTCGG |
| | CTCCGTAGGCGGACAACTTACTCCCGTATCTGCGCTCGCAGCCAGTACCCAC |
| | AAGCCATGGCTCGAGCAGCCACCTCGAGATCAAACTCTGACGTCATCAGATG |
| | AAGAGGACATTTACGCCCACGGGCTGCCCTCTTCTTCCTCAGAGACATCCGTT |
| | ACAGAGTTGGGTCCGTCTTGCTCACAACAAGATTTGTCAAGACTTGGTGCCG |
| | AAGATGCTGGTCTCCTGAAACCTGATCAGTTTGCAGAGTCCTGGATGGGCTA |
| | CTCTGGCCCGGGCTATGGGATACTCTCCCTGGTCGTCTCCGAAAAGTATATTT |
| | GGTGTCTTGATTATAAGGGGGGTCTCTTCTGTTCTGCATTGCCGGGAGCAGGC |
| | TTGAGATGGCAAAAATTCGAGGATGCAGTACAACAAGTAGCCGTATCACCTA |
| | GTGGAGCATTGCTGTGGAAGATTGAGCAAAAATCAAATCGGGCGTTTGCTTG |
| | TGGAAAGGTAACGATTAAAGGTAAGCGACACTGGTACGAAGCACTTCCCCAG |
| | GCCGTATTTGTTGCGCTGTCCGATGACACCGCCTGGATCATTCGAACATCTGG |
| | GGATTTGTATCTTCAAACGGGGCTCAGCGTAGATAGACCTTGTGCTCGGGCG |
| | GTAAAAGTGGACTGCCCATACCCCTTGTCACAGATTACTGCACGAAACAACG |
| | TCGTTTGGGCTCTGACGGAGCAACGAGCTCTGCTCTATAGGGAAGGTGTAAG |
| | CTCTTTCTGCCCAGAAGGGGAACAGTGGAAGTGTGATATCGTATCAGAGCGC |
| | CAAGCTCTCGAACCCGTATGTATCACACTGGGTGATCAGCAAACGCTTTGGG |
| | CTCTTGACATACATGGAAATCTTTGGTTTCGCACAGGCATCATTTCAAAAAAA |
| | CCACAGGGAGACGACGACCACTGGTGGCAAGTTTCTATCACGGATTATGTCG |
| | TATTCGATCAGTGTTCACTTTTCCAGACCATAATCCATGCCACGCATTCCGTG |
| | GCAACCGCGGCTCAAGCTCCCGTAGAAAAGGTGGCAGATAAACTCCGCATGG |
| | CATTTTGGTCACAACAGCTTCAGTGCCAACCTTCTTTGCTTGGTGTGAATAAC |
| | TCTGGAGTTTGGATTTCCAGTGGTAAAAACGAATTCCACGTTGCTAAGGGCA |
| | GCCTTATTGGTACTTATTGGAATCATGTTGTCCCTAGAGGAACAGCCTCTGCG |
| | ACCAAGTGGGCCTTCGTACTTGCATCAGCGGCGCCAACTAAGGAGGGTTCAT |
| | TCCTCTGGCTTTGCCAGTCTAGTAAAGACCTCTGTAGTGTGTCCGCACAAAGC |
| | GCCCAAAGCCGACCAAGCACCGTGCAGCTGCCGCCGGAGGCCGAGATGAGG |
| | GCGTACGCCGCATGTCAAGACGCTTTGTGGGCGCTTGATTCTCTGGGTCAGGT |
| | ATTTATTAGGACTCTTTCAAAGAGCTGCCCCACGGGCATGCACTGGACGCGG |
| | CTGGATCTCTCTCAGCTTGGGGCCGTAAAGCTCACTTCTCTTGCGTGTGGCAA |
| | CCAGCATATCTGGGCATGCGATTCCAGAGGGGGAGTATACTTTCGAGTGGGC |
| | ACACAACCTCTTAATCCGTCACTGATGCTTCCAGCGTGGATAATGATCGAGCC |
| | GCCGGTCCAACCCGCGGGTGTTTCACTTGTTAGTGTTCATTCATCTCCGAATG |
| | ACCAAATGCTCTGGGTCCTCGACTCTAGATGGAATGTTCACGTACGGACGGG |
| | TATTACAGAGGAGATGCCTGTAGGTACGGCTTGGGAGCATGTCCCTGGCCTG |
| | CAGGCATGTCAACTGGCTCTGTCCACTAGGACGGTCTGGGCCAGGTGTCCAA |
| | ATGGGGACCTTGCCAGACGGTACGGAGTGACAGACAAGAATCCTGCAGGTG |
| | ACTATTGGAAGAAAATACCCGGTAGTGTAAGCTGTTTCACCGTCACGGCCTC |
| | AGATGAACTGTGGGCTGTGGGACCACCTGGTTACCTCCTCCAGCGCTTGACT |
| | AAGACATTCTCCCATTCCCACGGCACCCAGAAAAGTTCACAAGCTGCTATGC |
| | CACATCCAGAAGACCTGGAAGATGAGTGGGAAGTTATTTGACCTTAACTTGT |
| | TTATTGCAGCTTATAATGGTTACAAATAAAGCAATAGCATCACAAATTTCAC |
| | AAATAAAGCATTTTTTTCACTGCATTCTAGTTGTGGTTTGTCCAAACTCATCA |
| | ATGTATCTTATCATGTCCATCGATAAGGATCTAGGAACCCCTAGTGATGGGC |
| | GCGCCATCGGATCCCGGGCCCGTCGACGCGCCGGCGTCTAGAAGGAACCCCT |
| | AGTGATGGAGTTGGCCACTCCCTCTCTGCGCGCTCGCTCGCTCACTGAGGCCG |
| | GGCGACCAAAGGTCGCCCGACGCCCGGGCTTTGCCCGGGCGGCCTCAGTGAG |
| | CGAGCGAGCGCGCAGAGAGGGAGTGGCCAACAGCTGCATTAATGAATCCTG |
| | CAGGCGGCCAACGCGCGGGGAGAGGCGGTTTGCGTATTGGGCGCTCTTCCGC |
| | TTCCTCGCTCACTGACTCGCTGCGCTCGGTCGTTCGGCTGCGGCGAGCGGTAT |
| | CAGCTCACTCAAAGGCGGTAATACGGTTATCCACAGAATCAGGGGATAACGC |
| | AGGAAAGAACATGTGAGCAAAAGGCCAGCAAAAGGCCAGGAACCGTAAAA |
| | AGGCCGCGTTGCTGGCGTTTTTCCATAGGCTCCGCCCCCCTGACGAGCATCAC |
| | AAAAATCGACGCTCAAGTCAGAGGTGGCGAAACCCGACAGGACTATAAAGA |
| | TACCAGGCGTTTCCCCCTGGAAGCTCCCTCGTGCGCTCTCCTGTTCCGACCCT |
| | GCCGCTTACCGGATACCTGTCCGCCTTTCTCCCTTCGGGAAGCGTGGCGCTTT |
| | CTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGG |
| | TAACTATCGTCTTGAGTCCAACCCGGTAAGACACGACTTATCGCCACTGGCA |
| | GCAGCCACTGGTAACAGGATTAGCAGAGCGAGGTATGTAGGCGGTGCTACA |
| | GAGTTCTTGAAGTGGTGGCCTAACTACGGCTACACTAGAAGAACAGTATTTG |
| | GTATCTGCGCTCTGCTGAAGCCAGTTACCTTCGGAAAAAGAGTTGGTAGCTCT |
| | TGATCCGGCAAACAAACCACCGCTGGTAGCGGTGGTTTTTTTGTTTGCAAGCA |
| | GCAGATTACGCGCAGAAAAAAAGGATCTCAAGAAGATCCTTTGATCTTTTCT |
| | ACGGGGTCTGACGCTCAGTGGAACGAAAACTCACGTTAAGGGATTTTGGTCA |
| | TGATTACCAATGCTTAATCAGTGAGGCACCTATCTCAGCGATCTGTCTATTTC |
| | GTTCATCCATAGTTGCCTGACTCCCCGTCGTGTAGATAACTACGATACGGGAG |
| | GGCTTACCATCTGGCCCCAGTGCTGCAATGATACCGCGAGACCCACGCTCAC |
| | CGGCTCCAGATTTATCAGCAATAAACCAGCCAGCCGGAAGGGCCGAGCGCA |
| | GAAGTGGTCCTGCAACTTTATCCGCCTCCATCCAGTCTATTAATTGTTGCCGG |
| | GAAGCTAGAGTAAGTAGTTCGCCAGTTAATAGTTTGCGCAACGTTGTTGCCA |
| | TTGCTACAGGCATCGTGGTGTCACGCTCGTCGTTTGGTATGGCTTCATTCAGC |
| | TCCGGTTCCCAACGATCAAGGCGAGTTACATGATCCCCCATGTTGTGCAAAA |
| | AAGCGGTTAGCTCCTTCGGTCCTCCGATCGTTGTCAGAAGTAAGTTGGCCGCA |
| | GTGTTATCACTCATGGTTATGGCAGCACTGCATAATTCTCTTACTGTCATGCC |
| | ATCCGTAAGATGCTTTTCTGTGACTGGTGAGTACTCAACCAAGTCATTCTGAG |
| | AATAGTGTATGCGGCGACCGAGTTGCTCTTGCCCGGCGTCAATACGGGATAA |
| | TACCGCGCCACATAGCAGAACTTTAAAAGTGCTCATCATTGGAAAACGTTCT |
| | TCGGGGCGAAAACTCTCAAGGATCTTACCGCTGTTGAGATCCAGTTCGATGT |
| | AACCCACTCGTGCACCCAACTGATCTTCAGCATCTTTTACTTTCACCAGCGTT |
| | TCTGGGTGAGCAAAAACAGGAAGGCAAAATGCCGCAAAAAAGGGAATAAGG |
| | GCGACACGGAAATGTTGAATACTCATACTCTTCCTTTTTCAATATTATTGAAG |
| | CATTTATCAGGGTTATTGTCTCTATGAGCGGATACATATTTGAATGTATTTAG |
| | AAAAATAAACAAATAGGGGTTCCGCGTCATGAGATTATCAAAAAGGATCTTC |
| | ACCTAGATCCTTTTCACGTAGAAAGCCAGTCCGCAGAAACGGTGCTGACCCC |
| | CTGTGGAATGTGTGTCAGTTAGGGTGTGGAAAGTCCCCAGGCTCCCCAGCAG |
| | GCAGAAGTATGCAAAGCATGCATCTCAATTAGTCAGCAACCAGGTGTGGAAA |
| | GTCCCCAGGCTCCCCAGCAGGCAGAAGTATGCAAAGCATGCATCTCAATTAG |
| | TCAGCAACCATAGTCCCGCCCCTAACTCCGCCCATCCCGCCCCTAACTCCGCC |
| | CAGTTCCGCCCATTCTCCGCCCCATGGCTGACTAATTTTTTTTATTTATGCAGA |
| | GGCCGAGGCCGCCTCGGCCTCTGAGCTATTCCAGAAGTAGTGAGGAGGCTTG |
| | GATGAATGTCAGCTACTGGGCTATCTGGACAAGGGAAAACGCAAGCGCAAA |
| | GAGAAAGCAGGTAGCTTGCAGTGGGCTTACATGGCGATAGCTAGACTGGGCG |
| | GTTTTATGGACAGCAAGCGAACCGGAATTGCCAGCTGGGGCGCCCTCTGGTA |
| | AGGTTGGGAAGCCCTGCAAAGTAAACTGGATGGCTTTCTTGCCGCCAAGGAT |
| | CTGATGGCGCAGGGGATCAAGCTCTGATCAAGAGACAGGATGAGGATCGTTT |
| | CGCATGATTGAACAAGATGGATTGCACGCAGGTTCTCCGGCCGCTTGGGTGG |
| | AGAGGCTATTCGGCTATGACTGGGCACAACAGACAATCGGCTGCTCTGATGC |
| | CGCCGTGTTCCGGCTGTCAGCGCAGGGGCGCCCGGTTCTTTTTGTCAAGACCG |
| | ACCTGTCCGGTGCCCTGAATGAACTGCAAGACGAGGCAGCGCGGCTATCGTG |
| | GCTGGCCACGACGGGCGTTCCTTGCGCAGCTGTGCTCGACGTTGTCACTGAA |
| | GCGGGAAGGGACTGGCTGCTATTGGGCGAAGTGCCGGGGCAGGATCTCCTGT |
| | CATCTCACCTTGCTCCTGCCGAGAAAGTATCCATCATGGCTGATGCAATGCGG |
| | CGGCTGCATACGCTTGATCCGGCTACCTGCCCATTCGACCACCAAGCGAAAC |
| | ATCGCATCGAGCGAGCACGTACTCGGATGGAAGCCGGTCTTGTCGATCAGGA |
| | TGATCTGGACGAAGAGCATCAGGGGCTCGCGCCAGCCGAACTGTTCGCCAGG |
| | CTCAAGGCGAGCATGCCCGACGGCGAGGATCTCGTCGTGACCCATGGCGATG |
| | CCTGCTTGCCGAATATCATGGTGGAAAATGGCCGCTTTTCTGGATTCATCGAC |
| | TGTGGCCGGCTGGGTGTGGCGGACCGCTATCAGGACATAGCGTTGGCTACCC |
| | GTGATATTGCTGAAGAGCTTGGCGGCGAATGGGCTGACCGCTTCCTCGTGCTT |
| | TACGGTATCGCCGCTCCCGATTCGCAGCGCATCGCCTTCTATCGCCTTCTTGA |
| | CGAGTTCTTCTGAATTTTCACACAAAAAACCAACACACAGATGTAATGAAAA |
| | TAAAGATATTTTATTGTTAAAATTTTTGTTAAATCAGCTCATTTTTTAACCAAT |
| | AGGCCGAAATCGGCAAAATCCCTTATAAATCAAAAGAATAGACCGAGATAG |
| | GGTTGAGTGTTGTTCCAGTTTGGAACAAGAGTCCACTATTAAAGAACGTGGA |
| | CTCCAACGTCAAAGGGCGAAAAACCGTCTATCAGGGCGATGGCCCACTACGT |
| | GAACCATCACCCTAATCAAGTTTTTTGGGGTCGAGGTGCCGTAAAGCACTAA |
| | ATCGGAACCCTAAAGGGAGCCCCCGATTTAGAGCTTGACGGGGAAAGCCGG |
| | CGAACGTGGCGAGAAAGGAAGGGAAGAAAGCGAAAGGAGCGGGCGCTAGG |
| | GCGCTGGCAAGTGTAGCGGTCACGCTGCGCGTAACCACCACACCCGCCGCGC |
| | TTAATGCGCCGCTACAGGGCGCGTCGCGTCCATTCGCCATTCAGGCTGCGCA |
| | ACTGTTGGGAAGGGCTGCAG |

TABLE 3
Nucleotide Sequence Information for TECPR2 Construct 2

| SEQ ID NO: | Nucleotide Sequence (5′→3′) |
|---|---|
| 2 | TTAATTAACTGCAGACGCCAGCTGTTGGCCACTCCCTCTCTGCGCGCTCGCTC |
| | GCTCACTGAGGCCGGGCGACCAAAGGTCGCCCGACGCCCGGGCTTTGCCCGG |
| | GCGGCCTCAGTGAGCGAGCGAGCGCGCAGAGAGGGAGTGGCCAACTCCATC |
| | ACTAGGGGTTCCTTCTAGAGGCGCGCCAAGCTTGGTACCCTAGGGCGGAGTT |
| | AGGGCGGAGCCAATCAGCGTGCGCCGTTCCGAAAGTTGCCTTTTATGGCTGG |
| | GCGGAGAATGGGCGGTGAACGCCGATGATTATATAAGGACGCGCCGGGTGT |
| | GGCACAGCTAGTTCCGTCGCAGCCGGGATTTGGGTCGCGGTTCTTGTTTGTGC |
| | GGCCGCCGCATGGCGAGCATCTCAGAGCCGGTCACGTTTAGAGAGTTCTGTC |
| | CACTTTATTACCTTCTTAATGCAATTCCAACTAAAATCCAGAAAGGATTTCGA |
| | TCTATAGTAGTTTATTTGACCGCATTGGATACGAACGGCGACTACATCGCGGT |
| | TGGCTCAAGCATCGGAATGCTCTATTTGTACTGTCGGCACCTCAATCAGATGA |
| | GGAAATATAACTTCGAAGGCAAGACCGAATCCATTACAGTTGTGAAGCTCCT |
| | CTCCTGTTTCGACGACCTGGTGGCCGCCGGCACCGCCTCCGGCCGAGTTGCA |
| | GTGTTCCAACTTGTATCAAGCCTCCCCGGCAGGAACAAGCAACTGAGAAGAT |
| | TTGACGTTACGGGTATTCATAAGAATTCCATAACGGCTCTGGCATGGAGCCC |
| | GAACGGGATGAAACTGTTCAGCGGTGACGATAAAGGGAAGATCGTGTACTCT |
| | TCTCTGGATCTGGACCAAGGGCTTTGCAACTCCCAACTTGTTCTCGAGGAACC |
| | ATCTTCTATCGTCCAGCTTGACTACAGTCAGAAGGTCTTGCTTGTGTCAACCC |
| | TTCAGCGAAGCCTTCTTTTTTATACGGAAGAGAAGTCCGTTCGGCAGATTGGG |
| | ACGCAACCCCGCAAAAGCACCGGTAAGTTTGGCGCATGTTTTATCCCAGGAC |
| | TGTGTAAACAATCTGACTTGACACTTTATGCCTCACGCCCAGGTCTGAGACTC |
| | TGGAAAGCCGACGTACACGGCACTGTACAAGCTACATTTATCTTGAAGGACG |
| | CTTTCGCCGGCGGCGTTAAGCCCTTCGAACTGCATCCCAGATTGGAGTCCCCC |
| | AATAGCGGTTCCTGTTCCCTTCCAGAGAGGCATCTGGGTCTTGTTTCCTGTTT |
| | CTTTCAGGAAGGCTGGGTGCTCAGCTGGAACGAATACTCAATATACCTGCTT |
| | GATACTGTGAACCAAGCTACTGTTGCGGGGCTGGAGGGAAGCGGTGATATTG |
| | TCAGCGTGTCATGCACTGAAAATGAGATTTTTTTTCTCAAAGGGGATCGAAAT |
| | ATAATTCGGATCTCATCCCGGCCCGAAGGACTTACTAGCACTGTAAGAGACG |
| | GACTGGAGATGAGCGGATGTTCCGAACGCGTGCACGTACAACAGGCTGAGA |
| | AGCTGCCCGGAGCTACTGTGAGTGAAACACGGTTGCGGGGCTCATCCATGGC |
| | TTCTTCAGTTGCAAGCGAACCGCGATCACGGTCATCATCATTGAACTCAACTG |
| | ATAGCGGCAGTGGACTGCTTCCCCCGGGATTGCAGGCTACCCCCGAACTCGG |
| | TAAAGGTTCTCAACCTCTCTCTCAGCGCTTCAACGCGATCTCCTCAGAAGATT |
| | TCGACCAGGAGTTGGTTGTTAAGCCCATCAAAGTCAAGCGCAAAAAGAAAA |
| | AAAAAAAGACCGAAGGGGGCTCCCGGAGTACGTGCCACTCCTCTCTTGAGAG |
| | TACTCCGTGTAGTGAGTTTCCAGGCGACAGTCCGCAGTCTCTGAACACAGAC |
| | CTTTTGAGTATGACAAGCTCAGTGCTTGGGAGCAGTGTAGACCAACTTTCAG |
| | CTGAGTCACCGGATCAGGAGTCTTCTTTCAATGGCGAGGTTAATGGTGTACC |
| | ACAGGAGAATACAGATCCCGAAACATTCAACGTCCTCGAAGTCTCTGGTAGT |
| | ATGCCAGATTCCCTCGCTGAAGAAGATGACATACGGACGGAAATGCCCCATT |
| | GCCACCACGCGCATGGTCGAGAGCTTCTTAACGGAGCAAGGGAAGACGTAG |
| | GGGGCTCCGACGTAACGGGGTTGGGTGATGAACCGTGCCCGGCTGACGACGG |
| | TCCCAATAGTACACAGTTGCCGTTCCAAGAACAGGACAGTTCCCCCGGCGCG |
| | CATGATGGAGAGGACATCCAGCCGATAGGTCCACAAAGTACTTTCTGCGAGG |
| | TTCCACTTCTCAACTCACTCACTGTCCCGAGCTCTCTTTCATGGGCCCCGTCA |
| | GCAGAACAATGGTTGCCAGGAACGCGCGCTGATGAAGGCTCCCCGGTTGAAC |
| | CATCCCAAGAGCAGGACATACTGACATCCATGGAAGCTAGTGGGCACCTTTC |
| | TACTAATCTGTGGCACGCGGTCACGGATGACGATACGGGTCAGAAGGAGATA |
| | CCAATCTCCGAGCGCGTACTCGGCTCCGTAGGCGGACAACTTACTCCCGTATC |
| | TGCGCTCGCAGCCAGTACCCACAAGCCATGGCTCGAGCAGCCACCTCGAGAT |
| | CAAACTCTGACGTCATCAGATGAAGAGGACATTTACGCCCACGGGCTGCCCT |
| | CTTCTTCCTCAGAGACATCCGTTACAGAGTTGGGTCCGTCTTGCTCACAACAA |
| | GATTTGTCAAGACTTGGTGCCGAAGATGCTGGTCTCCTGAAACCTGATCAGTT |
| | TGCAGAGTCCTGGATGGGCTACTCTGGCCCGGGCTATGGGATACTCTCCCTG |
| | GTCGTCTCCGAAAAGTATATTTGGTGTCTTGATTATAAGGGGGGTCTCTTCTG |
| | TTCTGCATTGCCGGGAGCAGGCTTGAGATGGCAAAAATTCGAGGATGCAGTA |
| | CAACAAGTAGCCGTATCACCTAGTGGAGCATTGCTGTGGAAGATTGAGCAAA |
| | AATCAAATCGGGCGTTTGCTTGTGGAAAGGTAACGATTAAAGGTAAGCGACA |
| | CTGGTACGAAGCACTTCCCCAGGCCGTATTTGTTGCGCTGTCCGATGACACCG |
| | CCTGGATCATTCGAACATCTGGGGATTTGTATCTTCAAACGGGGCTCAGCGTA |
| | GATAGACCTTGTGCTCGGGCGGTAAAAGTGGACTGCCCATACCCCTTGTCAC |
| | AGATTACTGCACGAAACAACGTCGTTTGGGCTCTGACGGAGCAACGAGCTCT |
| | GCTCTATAGGGAAGGTGTAAGCTCTTTCTGCCCAGAAGGGGAACAGTGGAAG |
| | TGTGATATCGTATCAGAGCGCCAAGCTCTCGAACCCGTATGTATCACACTGG |
| | GTGATCAGCAAACGCTTTGGGCTCTTGACATACATGGAAATCTTTGGTTTCGC |
| | ACAGGCATCATTTCAAAAAAACCACAGGGAGACGACGACCACTGGTGGCAA |
| | GTTTCTATCACGGATTATGTCGTATTCGATCAGTGTTCACTTTTCCAGACCAT |
| | AATCCATGCCACGCATTCCGTGGCAACCGCGGCTCAAGCTCCCGTAGAAAAG |
| | GTGGCAGATAAACTCCGCATGGCATTTTGGTCACAACAGCTTCAGTGCCAAC |
| | CTTCTTTGCTTGGTGTGAATAACTCTGGAGTTTGGATTTCCAGTGGTAAAAAC |
| | GAATTCCACGTTGCTAAGGGCAGCCTTATTGGTACTTATTGGAATCATGTTGT |
| | CCCTAGAGGAACAGCCTCTGCGACCAAGTGGGCCTTCGTACTTGCATCAGCG |
| | GCGCCAACTAAGGAGGGTTCATTCCTCTGGCTTTGCCAGTCTAGTAAAGACCT |
| | CTGTAGTGTGTCCGCACAAAGCGCCCAAAGCCGACCAAGCACCGTGCAGCTG |
| | CCGCCGGAGGCCGAGATGAGGGCGTACGCCGCATGTCAAGACGCTTTGTGGG |
| | CGCTTGATTCTCTGGGTCAGGTATTTATTAGGACTCTTTCAAAGAGCTGCCCC |
| | ACGGGCATGCACTGGACGCGGCTGGATCTCTCTCAGCTTGGGGCCGTAAAGC |
| | TCACTTCTCTTGCGTGTGGCAACCAGCATATCTGGGCATGCGATTCCAGAGGG |
| | GGAGTATACTTTCGAGTGGGCACACAACCTCTTAATCCGTCACTGATGCTTCC |
| | AGCGTGGATAATGATCGAGCCGCCGGTCCAACCCGCGGGTGTTTCACTTGTT |
| | AGTGTTCATTCATCTCCGAATGACCAAATGCTCTGGGTCCTCGACTCTAGATG |
| | GAATGTTCACGTACGGACGGGTATTACAGAGGAGATGCCTGTAGGTACGGCT |
| | TGGGAGCATGTCCCTGGCCTGCAGGCATGTCAACTGGCTCTGTCCACTAGGA |
| | CGGTCTGGGCCAGGTGTCCAAATGGGGACCTTGCCAGACGGTACGGAGTGAC |
| | AGACAAGAATCCTGCAGGTGACTATTGGAAGAAAATACCCGGTAGTGTAAGC |
| | TGTTTCACCGTCACGGCCTCAGATGAACTGTGGGCTGTGGGACCACCTGGTTA |
| | CCTCCTCCAGCGCTTGACTAAGACATTCTCCCATTCCCACGGCACCCAGAAAA |
| | GTTCACAAGCTGCTATGCCACATCCAGAAGACCTGGAAGATGAGTGGGAAGT |
| | TATTTGACCTTAACTTGTTTATTGCAGCTTATAATGGTTACAAATAAAGCAAT |
| | AGCATCACAAATTTCACAAATAAAGCATTTTTTTCACTGCATTCTAGTTGTGG |
| | TTTGTCCAAACTCATCAATGTATCTTATCATGTCCATCGATAAGGATCTAGGA |
| | ACCCCTAGTGATGGGCGCGCCATCGGATCCCGGGCCCGTCGACGCGCCGGCG |
| | TCTAGAAGGAACCCCTAGTGATGGAGTTGGCCACTCCCTCTCTGCGCGCTCGC |
| | TCGCTCACTGAGGCCGGGCGACCAAAGGTCGCCCGACGCCCGGGCTTTGCCC |
| | GGGCGGCCTCAGTGAGCGAGCGAGCGCGCAGAGAGGGAGTGGCCAACAGCT |
| | GCATTAATGAATCCTGCAGGCGGCCAACGCGCGGGGAGAGGCGGTTTGCGTA |
| | TTGGGCGCTCTTCCGCTTCCTCGCTCACTGACTCGCTGCGCTCGGTCGTTCGG |
| | CTGCGGCGAGCGGTATCAGCTCACTCAAAGGCGGTAATACGGTTATCCACAG |
| | AATCAGGGGATAACGCAGGAAAGAACATGTGAGCAAAAGGCCAGCAAAAGG |
| | CCAGGAACCGTAAAAAGGCCGCGTTGCTGGCGTTTTTCCATAGGCTCCGCCC |
| | CCCTGACGAGCATCACAAAAATCGACGCTCAAGTCAGAGGTGGCGAAACCC |
| | GACAGGACTATAAAGATACCAGGCGTTTCCCCCTGGAAGCTCCCTCGTGCGC |
| | TCTCCTGTTCCGACCCTGCCGCTTACCGGATACCTGTCCGCCTTTCTCCCTTCG |
| | GGAAGCGTGGCGCTTTCTCATAGCTCACGCTGTAGGTATCTCAGTTCGGTGTA |
| | GGTCGTTCGCTCCAAGCTGGGCTGTGTGCACGAACCCCCCGTTCAGCCCGAC |
| | CGCTGCGCCTTATCCGGTAACTATCGTCTTGAGTCCAACCCGGTAAGACACG |
| | ACTTATCGCCACTGGCAGCAGCCACTGGTAACAGGATTAGCAGAGCGAGGTA |
| | TGTAGGCGGTGCTACAGAGTTCTTGAAGTGGTGGCCTAACTACGGCTACACT |
| | AGAAGAACAGTATTTGGTATCTGCGCTCTGCTGAAGCCAGTTACCTTCGGAA |
| | AAAGAGTTGGTAGCTCTTGATCCGGCAAACAAACCACCGCTGGTAGCGGTGG |
| | TTTTTTTGTTTGCAAGCAGCAGATTACGCGCAGAAAAAAAGGATCTCAAGAA |
| | GATCCTTTGATCTTTTCTACGGGGTCTGACGCTCAGTGGAACGAAAACTCACG |
| | TTAAGGGATTTTGGTCATGATTACCAATGCTTAATCAGTGAGGCACCTATCTC |
| | AGCGATCTGTCTATTTCGTTCATCCATAGTTGCCTGACTCCCCGTCGTGTAGA |
| | TAACTACGATACGGGAGGGCTTACCATCTGGCCCCAGTGCTGCAATGATACC |
| | GCGAGACCCACGCTCACCGGCTCCAGATTTATCAGCAATAAACCAGCCAGCC |
| | GGAAGGGCCGAGCGCAGAAGTGGTCCTGCAACTTTATCCGCCTCCATCCAGT |
| | CTATTAATTGTTGCCGGGAAGCTAGAGTAAGTAGTTCGCCAGTTAATAGTTTG |
| | CGCAACGTTGTTGCCATTGCTACAGGCATCGTGGTGTCACGCTCGTCGTTTGG |
| | TATGGCTTCATTCAGCTCCGGTTCCCAACGATCAAGGCGAGTTACATGATCCC |
| | CCATGTTGTGCAAAAAAGCGGTTAGCTCCTTCGGTCCTCCGATCGTTGTCAGA |
| | AGTAAGTTGGCCGCAGTGTTATCACTCATGGTTATGGCAGCACTGCATAATTC |
| | TCTTACTGTCATGCCATCCGTAAGATGCTTTTCTGTGACTGGTGAGTACTCAA |
| | CCAAGTCATTCTGAGAATAGTGTATGCGGCGACCGAGTTGCTCTTGCCCGGC |
| | GTCAATACGGGATAATACCGCGCCACATAGCAGAACTTTAAAAGTGCTCATC |
| | ATTGGAAAACGTTCTTCGGGGCGAAAACTCTCAAGGATCTTACCGCTGTTGA |
| | GATCCAGTTCGATGTAACCCACTCGTGCACCCAACTGATCTTCAGCATCTTTT |
| | ACTTTCACCAGCGTTTCTGGGTGAGCAAAAACAGGAAGGCAAAATGCCGCAA |
| | AAAAGGGAATAAGGGCGACACGGAAATGTTGAATACTCATACTCTTCCTTTT |
| | TCAATATTATTGAAGCATTTATCAGGGTTATTGTCTCTATGAGCGGATACATA |
| | TTTGAATGTATTTAGAAAAATAAACAAATAGGGGTTCCGCGTCATGAGATTA |
| | TCAAAAAGGATCTTCACCTAGATCCTTTTCACGTAGAAAGCCAGTCCGCAGA |
| | AACGGTGCTGACCCCCTGTGGAATGTGTGTCAGTTAGGGTGTGGAAAGTCCC |
| | CAGGCTCCCCAGCAGGCAGAAGTATGCAAAGCATGCATCTCAATTAGTCAGC |
| | AACCAGGTGTGGAAAGTCCCCAGGCTCCCCAGCAGGCAGAAGTATGCAAAG |
| | CATGCATCTCAATTAGTCAGCAACCATAGTCCCGCCCCTAACTCCGCCCATCC |
| | CGCCCCTAACTCCGCCCAGTTCCGCCCATTCTCCGCCCCATGGCTGACTAATT |
| | TTTTTTATTTATGCAGAGGCCGAGGCCGCCTCGGCCTCTGAGCTATTCCAGAA |
| | GTAGTGAGGAGGCTTGGATGAATGTCAGCTACTGGGCTATCTGGACAAGGGA |
| | AAACGCAAGCGCAAAGAGAAAGCAGGTAGCTTGCAGTGGGCTTACATGGCG |
| | ATAGCTAGACTGGGCGGTTTTATGGACAGCAAGCGAACCGGAATTGCCAGCT |
| | GGGGCGCCCTCTGGTAAGGTTGGGAAGCCCTGCAAAGTAAACTGGATGGCTT |
| | TCTTGCCGCCAAGGATCTGATGGCGCAGGGGATCAAGCTCTGATCAAGAGAC |
| | AGGATGAGGATCGTTTCGCATGATTGAACAAGATGGATTGCACGCAGGTTCT |
| | CCGGCCGCTTGGGTGGAGAGGCTATTCGGCTATGACTGGGCACAACAGACAA |
| | TCGGCTGCTCTGATGCCGCCGTGTTCCGGCTGTCAGCGCAGGGGCGCCCGGTT |
| | CTTTTTGTCAAGACCGACCTGTCCGGTGCCCTGAATGAACTGCAAGACGAGG |
| | CAGCGCGGCTATCGTGGCTGGCCACGACGGGCGTTCCTTGCGCAGCTGTGCT |
| | CGACGTTGTCACTGAAGCGGGAAGGGACTGGCTGCTATTGGGCGAAGTGCCG |
| | GGGCAGGATCTCCTGTCATCTCACCTTGCTCCTGCCGAGAAAGTATCCATCAT |
| | GGCTGATGCAATGCGGCGGCTGCATACGCTTGATCCGGCTACCTGCCCATTC |
| | GACCACCAAGCGAAACATCGCATCGAGCGAGCACGTACTCGGATGGAAGCC |
| | GGTCTTGTCGATCAGGATGATCTGGACGAAGAGCATCAGGGGCTCGCGCCAG |
| | CCGAACTGTTCGCCAGGCTCAAGGCGAGCATGCCCGACGGCGAGGATCTCGT |
| | CGTGACCCATGGCGATGCCTGCTTGCCGAATATCATGGTGGAAAATGGCCGC |
| | TTTTCTGGATTCATCGACTGTGGCCGGCTGGGTGTGGCGGACCGCTATCAGGA |
| | CATAGCGTTGGCTACCCGTGATATTGCTGAAGAGCTTGGCGGCGAATGGGCT |
| | GACCGCTTCCTCGTGCTTTACGGTATCGCCGCTCCCGATTCGCAGCGCATCGC |
| | CTTCTATCGCCTTCTTGACGAGTTCTTCTGAATTTTCACACAAAAAACCAACA |
| | CACAGATGTAATGAAAATAAAGATATTTTATTGTTAAAATTTTTGTTAAATCA |
| | GCTCATTTTTTAACCAATAGGCCGAAATCGGCAAAATCCCTTATAAATCAAA |
| | AGAATAGACCGAGATAGGGTTGAGTGTTGTTCCAGTTTGGAACAAGAGTCCA |
| | CTATTAAAGAACGTGGACTCCAACGTCAAAGGGCGAAAAACCGTCTATCAGG |
| | GCGATGGCCCACTACGTGAACCATCACCCTAATCAAGTTTTTTGGGGTCGAG |
| | GTGCCGTAAAGCACTAAATCGGAACCCTAAAGGGAGCCCCCGATTTAGAGCT |
| | TGACGGGGAAAGCCGGCGAACGTGGCGAGAAAGGAAGGGAAGAAAGCGAA |
| | AGGAGCGGGCGCTAGGGCGCTGGCAAGTGTAGCGGTCACGCTGCGCGTAAC |
| | CACCACACCCGCCGCGCTTAATGCGCCGCTACAGGGCGCGTCGCGTCCATTC |
| | GCCATTCAGGCTGCGCAACTGTTGGGAAGGGCTGCAG |

TABLE 4
Nucleotide Sequence Information for TECPR2 Construct 3

| SEQ ID NO: | Nucleotide Sequence (5′→3′) |
|---|---|
| 3 | TTAATTAACTGCAGACGCCAGCTGTTGGCCACTCCCTCTCTGCGCGCTCGCTC |
| | GCTCACTGAGGCCGGGCGACCAAAGGTCGCCCGACGCCCGGGCTTTGCCCGG |
| | GCGGCCTCAGTGAGCGAGCGAGCGCGCAGAGAGGGAGTGGCCAACTCCATC |
| | ACTAGGGGTTCCTTCTAGAGGCGCGCCAAGCTTGGTACCGCTGCAGAGGGCC |
| | CTGCGTATGAGTGCAAGTGGGTTTTAGGACCAGGATGAGGCGGGGTGGGGGT |
| | GCCTACCTGACGACCGACCCCGACCCACTGGACAAGCACCCAACCCCCATTC |
| | CCCAAATTGCGCATCCCCTATCAGAGAGGGGGAGGGGAAACAGGATGCGGC |
| | GAGGCGCGTGCGCACTGCCAGCTTCAGCACCGCGGACAGTGCCTTCGCCCCC |
| | GCCTGGCGGCGCGCGCCACCGCCGCCTCAGCACTGAAGGCGCGCTGACGTCA |
| | CTCGCCGGTCCCCCGCAAACTCCCCTTCCCGGCCACCTTGGTCGCGTCCGCGC |
| | CGCCGCCGGCCCAGCCGGACCGCACCACGCGAGGCGCGAGATAGGGGGGCA |
| | CGGGCGCGACCATCTGCGCTGCGGCGCCGGCGTCGACAAGAGGTAAGGGTTT |
| | AAGGGATGGTTGGTTGGTGGGGTATTAATGTTTAATTACCTGTTTTACAGGCC |
| | TGAAATCACTTGGTTTTAGGTTGGGAAGCTTGATATCCCTGCAGGGCGGCCG |
| | CCGCATGGCGAGCATCTCAGAGCCGGTCACGTTTAGAGAGTTCTGTCCACTTT |
| | ATTACCTTCTTAATGCAATTCCAACTAAAATCCAGAAAGGATTTCGATCTATA |
| | GTAGTTTATTTGACCGCATTGGATACGAACGGCGACTACATCGCGGTTGGCTC |
| | AAGCATCGGAATGCTCTATTTGTACTGTCGGCACCTCAATCAGATGAGGAAA |
| | TATAACTTCGAAGGCAAGACCGAATCCATTACAGTIGTGAAGCTCCTCTCCTG |
| | TTTCGACGACCTGGTGGCCGCCGGCACCGCCTCCGGCCGAGTTGCAGTGTTCC |
| | AACTTGTATCAAGCCTCCCCGGCAGGAACAAGCAACTGAGAAGATTTGACGT |
| | TACGGGTATTCATAAGAATTCCATAACGGCTCTGGCATGGAGCCCGAACGGG |
| | ATGAAACTGTTCAGCGGTGACGATAAAGGGAAGATCGTGTACTCTTCTCTGG |
| | ATCTGGACCAAGGGCTTTGCAACTCCCAACTTGTTCTCGAGGAACCATCTTCT |
| | ATCGTCCAGCTTGACTACAGTCAGAAGGTCTTGCTTGTGTCAACCCTTCAGCG |
| | AAGCCTTCTTTTTTATACGGAAGAGAAGTCCGTTCGGCAGATTGGGACGCAA |
| | CCCCGCAAAAGCACCGGTAAGTTTGGCGCATGTTTTATCCCAGGACTGTGTA |
| | AACAATCTGACTTGACACTTTATGCCTCACGCCCAGGTCTGAGACTCTGGAA |
| | AGCCGACGTACACGGCACTGTACAAGCTACATTTATCTTGAAGGACGCTTTC |
| | GCCGGCGGCGTTAAGCCCTTCGAACTGCATCCCAGATTGGAGTCCCCCAATA |
| | GCGGTTCCTGTTCCCTTCCAGAGAGGCATCTGGGTCTTGTTTCCTGTTTCTTTC |
| | AGGAAGGCTGGGTGCTCAGCTGGAACGAATACTCAATATACCTGCTTGATAC |
| | TGTGAACCAAGCTACTGTTGCGGGGCTGGAGGGAAGCGGTGATATTGTCAGC |
| | GTGTCATGCACTGAAAATGAGATTTTTTTTCTCAAAGGGGATCGAAATATAAT |
| | TCGGATCTCATCCCGGCCCGAAGGACTTACTAGCACTGTAAGAGACGGACTG |
| | GAGATGAGCGGATGTTCCGAACGCGTGCACGTACAACAGGCTGAGAAGCTG |
| | CCCGGAGCTACTGTGAGTGAAACACGGTTGCGGGGCTCATCCATGGCTTCTT |
| | CAGTTGCAAGCGAACCGCGATCACGGTCATCATCATTGAACTCAACTGATAG |
| | CGGCAGTGGACTGCTTCCCCCGGGATTGCAGGCTACCCCCGAACTCGGTAAA |
| | GGTTCTCAACCTCTCTCTCAGCGCTTCAACGCGATCTCCTCAGAAGATTTCGA |
| | CCAGGAGTTGGTTGTTAAGCCCATCAAAGTCAAGCGCAAAAAGAAAAAAAA |
| | AAAGACCGAAGGGGGCTCCCGGAGTACGTGCCACTCCTCTCTTGAGAGTACT |
| | CCGTGTAGTGAGTTTCCAGGCGACAGTCCGCAGTCTCTGAACACAGACCTTTT |
| | GAGTATGACAAGCTCAGTGCTTGGGAGCAGTGTAGACCAACTTTCAGCTGAG |
| | TCACCGGATCAGGAGTCTTCTTTCAATGGCGAGGTTAATGGTGTACCACAGG |
| | AGAATACAGATCCCGAAACATTCAACGTCCTCGAAGTCTCTGGTAGTATGCC |
| | AGATTCCCTCGCTGAAGAAGATGACATACGGACGGAAATGCCCCATTGCCAC |
| | CACGCGCATGGTCGAGAGCTTCTTAACGGAGCAAGGGAAGACGTAGGGGGC |
| | TCCGACGTAACGGGGTTGGGTGATGAACCGTGCCCGGCTGACGACGGTCCCA |
| | ATAGTACACAGTTGCCGTTCCAAGAACAGGACAGTTCCCCCGGCGCGCATGA |
| | TGGAGAGGACATCCAGCCGATAGGTCCACAAAGTACTTTCTGCGAGGTTCCA |
| | CTTCTCAACTCACTCACTGTCCCGAGCTCTCTTTCATGGGCCCCGTCAGCAGA |
| | ACAATGGTTGCCAGGAACGCGCGCTGATGAAGGCTCCCCGGTTGAACCATCC |
| | CAAGAGCAGGACATACTGACATCCATGGAAGCTAGTGGGCACCTTTCTACTA |
| | ATCTGTGGCACGCGGTCACGGATGACGATACGGGTCAGAAGGAGATACCAAT |
| | CTCCGAGCGCGTACTCGGCTCCGTAGGCGGACAACTTACTCCCGTATCTGCGC |
| | TCGCAGCCAGTACCCACAAGCCATGGCTCGAGCAGCCACCTCGAGATCAAAC |
| | TCTGACGTCATCAGATGAAGAGGACATTTACGCCCACGGGCTGCCCTCTTCTT |
| | CCTCAGAGACATCCGTTACAGAGTTGGGTCCGTCTTGCTCACAACAAGATTTG |
| | TCAAGACTTGGTGCCGAAGATGCTGGTCTCCTGAAACCTGATCAGTTTGCAG |
| | AGTCCTGGATGGGCTACTCTGGCCCGGGCTATGGGATACTCTCCCTGGTCGTC |
| | TCCGAAAAGTATATTTGGTGTCTTGATTATAAGGGGGGTCTCTTCTGTTCTGC |
| | ATTGCCGGGAGCAGGCTTGAGATGGCAAAAATTCGAGGATGCAGTACAACA |
| | AGTAGCCGTATCACCTAGTGGAGCATTGCTGTGGAAGATTGAGCAAAAATCA |
| | AATCGGGCGTTTGCTTGTGGAAAGGTAACGATTAAAGGTAAGCGACACTGGT |
| | ACGAAGCACTTCCCCAGGCCGTATTTGTTGCGCTGTCCGATGACACCGCCTGG |
| | ATCATTCGAACATCTGGGGATTTGTATCTTCAAACGGGGCTCAGCGTAGATA |
| | GACCTTGTGCTCGGGCGGTAAAAGTGGACTGCCCATACCCCTTGTCACAGAT |
| | TACTGCACGAAACAACGTCGTTTGGGCTCTGACGGAGCAACGAGCTCTGCTC |
| | TATAGGGAAGGTGTAAGCTCTTTCTGCCCAGAAGGGGAACAGTGGAAGTGTG |
| | ATATCGTATCAGAGCGCCAAGCTCTCGAACCCGTATGTATCACACTGGGTGA |
| | TCAGCAAACGCTTTGGGCTCTTGACATACATGGAAATCTTTGGTTTCGCACAG |
| | GCATCATTTCAAAAAAACCACAGGGAGACGACGACCACTGGTGGCAAGTTTC |
| | TATCACGGATTATGTCGTATTCGATCAGTGTTCACTTTTCCAGACCATAATCC |
| | ATGCCACGCATTCCGTGGCAACCGCGGCTCAAGCTCCCGTAGAAAAGGTGGC |
| | AGATAAACTCCGCATGGCATTTTGGTCACAACAGCTTCAGTGCCAACCTTCTT |
| | TGCTTGGTGTGAATAACTCTGGAGTTTGGATTTCCAGTGGTAAAAACGAATTC |
| | CACGTTGCTAAGGGCAGCCTTATTGGTACTTATTGGAATCATGTTGTCCCTAG |
| | AGGAACAGCCTCTGCGACCAAGTGGGCCTTCGTACTTGCATCAGCGGCGCCA |
| | ACTAAGGAGGGTTCATTCCTCTGGCTTTGCCAGTCTAGTAAAGACCTCTGTAG |
| | TGTGTCCGCACAAAGCGCCCAAAGCCGACCAAGCACCGTGCAGCTGCCGCCG |
| | GAGGCCGAGATGAGGGCGTACGCCGCATGTCAAGACGCTTTGTGGGCGCTTG |
| | ATTCTCTGGGTCAGGTATTTATTAGGACTCTTTCAAAGAGCTGCCCCACGGGC |
| | ATGCACTGGACGCGGCTGGATCTCTCTCAGCTTGGGGCCGTAAAGCTCACTTC |
| | TCTTGCGTGTGGCAACCAGCATATCTGGGCATGCGATTCCAGAGGGGGAGTA |
| | TACTTTCGAGTGGGCACACAACCTCTTAATCCGTCACTGATGCTTCCAGCGTG |
| | GATAATGATCGAGCCGCCGGTCCAACCCGCGGGTGTTTCACTTGTTAGTGTTC |
| | ATTCATCTCCGAATGACCAAATGCTCTGGGTCCTCGACTCTAGATGGAATGTT |
| | CACGTACGGACGGGTATTACAGAGGAGATGCCTGTAGGTACGGCTTGGGAGC |
| | ATGTCCCTGGCCTGCAGGCATGTCAACTGGCTCTGTCCACTAGGACGGTCTGG |
| | GCCAGGTGTCCAAATGGGGACCTTGCCAGACGGTACGGAGTGACAGACAAG |
| | AATCCTGCAGGTGACTATTGGAAGAAAATACCCGGTAGTGTAAGCTGTTTCA |
| | CCGTCACGGCCTCAGATGAACTGTGGGCTGTGGGACCACCTGGTTACCTCCTC |
| | CAGCGCTTGACTAAGACATTCTCCCATTCCCACGGCACCCAGAAAAGTTCAC |
| | AAGCTGCTATGCCACATCCAGAAGACCTGGAAGATGAGTGGGAAGTTATTTG |
| | ACCTTAACTTGTTTATTGCAGCTTATAATGGTTACAAATAAAGCAATAGCATC |
| | ACAAATTTCACAAATAAAGCATTTTTTTCACTGCATTCTAGTTGTGGTTTGTC |
| | CAAACTCATCAATGTATCTTATCATGTCCATCGATAAGGATCTAGGAACCCCT |
| | AGTGATGGGCGCGCCATCGGATCCCGGGCCCGTCGACGCGCCGGCGTCTAGA |
| | AGGAACCCCTAGTGATGGAGTTGGCCACTCCCTCTCTGCGCGCTCGCTCGCTC |
| | ACTGAGGCCGGGCGACCAAAGGTCGCCCGACGCCCGGGCTTTGCCCGGGCGG |
| | CCTCAGTGAGCGAGCGAGCGCGCAGAGAGGGAGTGGCCAACAGCTGCATTA |
| | ATGAATCCTGCAGGCGGCCAACGCGCGGGGAGAGGCGGTTTGCGTATTGGGC |
| | GCTCTTCCGCTTCCTCGCTCACTGACTCGCTGCGCTCGGTCGTTCGGCTGCGG |
| | CGAGCGGTATCAGCTCACTCAAAGGCGGTAATACGGTTATCCACAGAATCAG |
| | GGGATAACGCAGGAAAGAACATGTGAGCAAAAGGCCAGCAAAAGGCCAGG |
| | AACCGTAAAAAGGCCGCGTTGCTGGCGTTTTTCCATAGGCTCCGCCCCCCTGA |
| | CGAGCATCACAAAAATCGACGCTCAAGTCAGAGGTGGCGAAACCCGACAGG |
| | ACTATAAAGATACCAGGCGTTTCCCCCTGGAAGCTCCCTCGTGCGCTCTCCTG |
| | TTCCGACCCTGCCGCTTACCGGATACCTGTCCGCCTTTCTCCCTTCGGGAAGC |
| | GTGGCGCTTTCTCATAGCTCACGCTGTAGGTATCTCAGTTCGGTGTAGGTCGT |
| | TCGCTCCAAGCTGGGCTGTGTGCACGAACCCCCCGTTCAGCCCGACCGCTGC |
| | GCCTTATCCGGTAACTATCGTCTTGAGTCCAACCCGGTAAGACACGACTTATC |
| | GCCACTGGCAGCAGCCACTGGTAACAGGATTAGCAGAGCGAGGTATGTAGG |
| | CGGTGCTACAGAGTTCTTGAAGTGGTGGCCTAACTACGGCTACACTAGAAGA |
| | ACAGTATTTGGTATCTGCGCTCTGCTGAAGCCAGTTACCTTCGGAAAAAGAG |
| | TTGGTAGCTCTTGATCCGGCAAACAAACCACCGCTGGTAGCGGTGGTTTTTTT |
| | GTTTGCAAGCAGCAGATTACGCGCAGAAAAAAAGGATCTCAAGAAGATCCTT |
| | TGATCTTTTCTACGGGGTCTGACGCTCAGTGGAACGAAAACTCACGTTAAGG |
| | GATTTTGGTCATGATTACCAATGCTTAATCAGTGAGGCACCTATCTCAGCGAT |
| | CTGTCTATTTCGTTCATCCATAGTTGCCTGACTCCCCGTCGTGTAGATAACTA |
| | CGATACGGGAGGGCTTACCATCTGGCCCCAGTGCTGCAATGATACCGCGAGA |
| | CCCACGCTCACCGGCTCCAGATTTATCAGCAATAAACCAGCCAGCCGGAAGG |
| | GCCGAGCGCAGAAGTGGTCCTGCAACTTTATCCGCCTCCATCCAGTCTATTAA |
| | TTGTTGCCGGGAAGCTAGAGTAAGTAGTTCGCCAGTTAATAGTTTGCGCAAC |
| | GTTGTTGCCATTGCTACAGGCATCGTGGTGTCACGCTCGTCGTTTGGTATGGC |
| | TTCATTCAGCTCCGGTTCCCAACGATCAAGGCGAGTTACATGATCCCCCATGT |
| | TGTGCAAAAAAGCGGTTAGCTCCTTCGGTCCTCCGATCGTTGTCAGAAGTAA |
| | GTTGGCCGCAGTGTTATCACTCATGGTTATGGCAGCACTGCATAATTCTCTTA |
| | CTGTCATGCCATCCGTAAGATGCTTTTCTGTGACTGGTGAGTACTCAACCAAG |
| | TCATTCTGAGAATAGTGTATGCGGCGACCGAGTTGCTCTTGCCCGGCGTCAAT |
| | ACGGGATAATACCGCGCCACATAGCAGAACTTTAAAAGTGCTCATCATTGGA |
| | AAACGTTCTTCGGGGCGAAAACTCTCAAGGATCTTACCGCTGTTGAGATCCA |
| | GTTCGATGTAACCCACTCGTGCACCCAACTGATCTTCAGCATCTTTTACTTTC |
| | ACCAGCGTTTCTGGGTGAGCAAAAACAGGAAGGCAAAATGCCGCAAAAAAG |
| | GGAATAAGGGCGACACGGAAATGTTGAATACTCATACTCTTCCTTTTTCAATA |
| | TTATTGAAGCATTTATCAGGGTTATTGTCTCTATGAGCGGATACATATTTGAA |
| | TGTATTTAGAAAAATAAACAAATAGGGGTTCCGCGTCATGAGATTATCAAAA |
| | AGGATCTTCACCTAGATCCTTTTCACGTAGAAAGCCAGTCCGCAGAAACGGT |
| | GCTGACCCCCTGTGGAATGTGTGTCAGTTAGGGTGTGGAAAGTCCCCAGGCT |
| | CCCCAGCAGGCAGAAGTATGCAAAGCATGCATCTCAATTAGTCAGCAACCAG |
| | GTGTGGAAAGTCCCCAGGCTCCCCAGCAGGCAGAAGTATGCAAAGCATGCAT |
| | CTCAATTAGTCAGCAACCATAGTCCCGCCCCTAACTCCGCCCATCCCGCCCCT |
| | AACTCCGCCCAGTTCCGCCCATTCTCCGCCCCATGGCTGACTAATTTTTTTTAT |
| | TTATGCAGAGGCCGAGGCCGCCTCGGCCTCTGAGCTATTCCAGAAGTAGTGA |
| | GGAGGCTTGGATGAATGTCAGCTACTGGGCTATCTGGACAAGGGAAAACGCA |
| | AGCGCAAAGAGAAAGCAGGTAGCTTGCAGTGGGCTTACATGGCGATAGCTA |
| | GACTGGGCGGTTTTATGGACAGCAAGCGAACCGGAATTGCCAGCTGGGGCGC |
| | CCTCTGGTAAGGTTGGGAAGCCCTGCAAAGTAAACTGGATGGCTTTCTTGCC |
| | GCCAAGGATCTGATGGCGCAGGGGATCAAGCTCTGATCAAGAGACAGGATG |
| | AGGATCGTTTCGCATGATTGAACAAGATGGATTGCACGCAGGTTCTCCGGCC |
| | GCTTGGGTGGAGAGGCTATTCGGCTATGACTGGGCACAACAGACAATCGGCT |
| | GCTCTGATGCCGCCGTGTTCCGGCTGTCAGCGCAGGGGCGCCCGGTTCTTTTT |
| | GTCAAGACCGACCTGTCCGGTGCCCTGAATGAACTGCAAGACGAGGCAGCGC |
| | GGCTATCGTGGCTGGCCACGACGGGCGTTCCTTGCGCAGCTGTGCTCGACGTT |
| | GTCACTGAAGCGGGAAGGGACTGGCTGCTATTGGGCGAAGTGCCGGGGCAG |
| | GATCTCCTGTCATCTCACCTTGCTCCTGCCGAGAAAGTATCCATCATGGCTGA |
| | TGCAATGCGGCGGCTGCATACGCTTGATCCGGCTACCTGCCCATTCGACCACC |
| | AAGCGAAACATCGCATCGAGCGAGCACGTACTCGGATGGAAGCCGGTCTTGT |
| | CGATCAGGATGATCTGGACGAAGAGCATCAGGGGCTCGCGCCAGCCGAACT |
| | GTTCGCCAGGCTCAAGGCGAGCATGCCCGACGGCGAGGATCTCGTCGTGACC |
| | CATGGCGATGCCTGCTTGCCGAATATCATGGTGGAAAATGGCCGCTTTTCTGG |
| | ATTCATCGACTGTGGCCGGCTGGGTGTGGCGGACCGCTATCAGGACATAGCG |
| | TTGGCTACCCGTGATATTGCTGAAGAGCTTGGCGGCGAATGGGCTGACCGCT |
| | TCCTCGTGCTTTACGGTATCGCCGCTCCCGATTCGCAGCGCATCGCCTTCTAT |
| | CGCCTTCTTGACGAGTTCTTCTGAATTTTCACACAAAAAACCAACACACAGAT |
| | GTAATGAAAATAAAGATATTTTATTGTTAAAATTTTTGTTAAATCAGCTCATT |
| | TTTTAACCAATAGGCCGAAATCGGCAAAATCCCTTATAAATCAAAAGAATAG |
| | ACCGAGATAGGGTTGAGTGTTGTTCCAGTTTGGAACAAGAGTCCACTATTAA |
| | AGAACGTGGACTCCAACGTCAAAGGGCGAAAAACCGTCTATCAGGGCGATG |
| | GCCCACTACGTGAACCATCACCCTAATCAAGTTTTTTGGGGTCGAGGTGCCGT |
| | AAAGCACTAAATCGGAACCCTAAAGGGAGCCCCCGATTTAGAGCTTGACGGG |
| | GAAAGCCGGCGAACGTGGCGAGAAAGGAAGGGAAGAAAGCGAAAGGAGCG |
| | GGCGCTAGGGCGCTGGCAAGTGTAGCGGTCACGCTGCGCGTAACCACCACAC |
| | CCGCCGCGCTTAATGCGCCGCTACAGGGCGCGTCGCGTCCATTCGCCATTCA |
| | GGCTGCGCAACTGTTGGGAAGGGCTGCAG |

TABLE 5
Nucleotide Sequence Information for TECPR2 Construct 4

| SEQ ID NO: | Nucleotide Sequence (5′→3′) |
|---|---|
| 4 | TTAATTAACTGCAGACGCCAGCTGTTGGCCACTCCCTCTCTGCGCGCTCGCTC |
| | GCTCACTGAGGCCGGGCGACCAAAGGTCGCCCGACGCCCGGGCTTTGCCCGG |
| | GCGGCCTCAGTGAGCGAGCGAGCGCGCAGAGAGGGAGTGGCCAACTCCATC |
| | ACTAGGGGTTCCTTCTAGAGGCGCGCCAAGCTTGGTACCAAAATCAACGGGA |
| | CTTTCCAAAATGTCGTAATAACCCCGCCCCGTTGACGCAAATGGGCGGTAGG |
| | CGTGTACGGTGGGAGGTCTATATAAGCAGAGCTCGTTTAGTGAACCGTCAGA |
| | ATTCTCGAGTGATCGAAAGAGCCTGCTAAAGCAAAAAAGAAGTCACCGTCGA |
| | CAAGAGGTAAGGGTTTAAGGGATGGTTGGTTGGTGGGGTATTAATGTTTAAT |
| | TACCTGTTTTACAGGCCTGAAATCACTTGGTTTTAGGTTGGGAAGCTTGATAT |
| | CCCTGCAGGGCGGCCGCCGCATGGCGAGCATCTCAGAGCCGGTCACGTTTAG |
| | AGAGTTCTGTCCACTTTATTACCTTCTTAATGCAATTCCAACTAAAATCCAGA |
| | AAGGATTTCGATCTATAGTAGTTTATTTGACCGCATTGGATACGAACGGCGA |
| | CTACATCGCGGTTGGCTCAAGCATCGGAATGCTCTATTTGTACTGTCGGCACC |
| | TCAATCAGATGAGGAAATATAACTTCGAAGGCAAGACCGAATCCATTACAGT |
| | TGTGAAGCTCCTCTCCTGTTTCGACGACCTGGTGGCCGCCGGCACCGCCTCCG |
| | GCCGAGTTGCAGTGTTCCAACTTGTATCAAGCCTCCCCGGCAGGAACAAGCA |
| | ACTGAGAAGATTTGACGTTACGGGTATTCATAAGAATTCCATAACGGCTCTG |
| | GCATGGAGCCCGAACGGGATGAAACTGTTCAGCGGTGACGATAAAGGGAAG |
| | ATCGTGTACTCTTCTCTGGATCTGGACCAAGGGCTTTGCAACTCCCAACTTGT |
| | TCTCGAGGAACCATCTTCTATCGTCCAGCTTGACTACAGTCAGAAGGTCTTGC |
| | TTGTGTCAACCCTTCAGCGAAGCCTTCTTTTTTATACGGAAGAGAAGTCCGTT |
| | CGGCAGATTGGGACGCAACCCCGCAAAAGCACCGGTAAGTTTGGCGCATGTT |
| | TTATCCCAGGACTGTGTAAACAATCTGACTTGACACTTTATGCCTCACGCCCA |
| | GGTCTGAGACTCTGGAAAGCCGACGTACACGGCACTGTACAAGCTACATTTA |
| | TCTTGAAGGACGCTTTCGCCGGCGGCGTTAAGCCCTTCGAACTGCATCCCAG |
| | ATTGGAGTCCCCCAATAGCGGTTCCTGTTCCCTTCCAGAGAGGCATCTGGGTC |
| | TTGTTTCCTGTTTCTTTCAGGAAGGCTGGGTGCTCAGCTGGAACGAATACTCA |
| | ATATACCTGCTTGATACTGTGAACCAAGCTACTGTTGCGGGGCTGGAGGGAA |
| | GCGGTGATATTGTCAGCGTGTCATGCACTGAAAATGAGATTTTTTTTCTCAAA |
| | GGGGATCGAAATATAATTCGGATCTCATCCCGGCCCGAAGGACTTACTAGCA |
| | CTGTAAGAGACGGACTGGAGATGAGCGGATGTTCCGAACGCGTGCACGTACA |
| | ACAGGCTGAGAAGCTGCCCGGAGCTACTGTGAGTGAAACACGGTTGCGGGG |
| | CTCATCCATGGCTTCTTCAGTTGCAAGCGAACCGCGATCACGGTCATCATCAT |
| | TGAACTCAACTGATAGCGGCAGTGGACTGCTTCCCCCGGGATTGCAGGCTAC |
| | CCCCGAACTCGGTAAAGGTTCTCAACCTCTCTCTCAGCGCTTCAACGCGATCT |
| | CCTCAGAAGATTTCGACCAGGAGTTGGTTGTTAAGCCCATCAAAGTCAAGCG |
| | CAAAAAGAAAAAAAAAAAGACCGAAGGGGGCTCCCGGAGTACGTGCCACTC |
| | CTCTCTTGAGAGTACTCCGTGTAGTGAGTTTCCAGGCGACAGTCCGCAGTCTC |
| | TGAACACAGACCTTTTGAGTATGACAAGCTCAGTGCTTGGGAGCAGTGTAGA |
| | CCAACTTTCAGCTGAGTCACCGGATCAGGAGTCTTCTTTCAATGGCGAGGTTA |
| | ATGGTGTACCACAGGAGAATACAGATCCCGAAACATTCAACGTCCTCGAAGT |
| | CTCTGGTAGTATGCCAGATTCCCTCGCTGAAGAAGATGACATACGGACGGAA |
| | ATGCCCCATTGCCACCACGCGCATGGTCGAGAGCTTCTTAACGGAGCAAGGG |
| | AAGACGTAGGGGGCTCCGACGTAACGGGGTTGGGTGATGAACCGTGCCCGG |
| | CTGACGACGGTCCCAATAGTACACAGTTGCCGTTCCAAGAACAGGACAGTTC |
| | CCCCGGCGCGCATGATGGAGAGGACATCCAGCCGATAGGTCCACAAAGTACT |
| | TTCTGCGAGGTTCCACTTCTCAACTCACTCACTGTCCCGAGCTCTCTTTCATGG |
| | GCCCCGTCAGCAGAACAATGGTTGCCAGGAACGCGCGCTGATGAAGGCTCCC |
| | CGGTTGAACCATCCCAAGAGCAGGACATACTGACATCCATGGAAGCTAGTGG |
| | GCACCTTTCTACTAATCTGTGGCACGCGGTCACGGATGACGATACGGGTCAG |
| | AAGGAGATACCAATCTCCGAGCGCGTACTCGGCTCCGTAGGCGGACAACTTA |
| | CTCCCGTATCTGCGCTCGCAGCCAGTACCCACAAGCCATGGCTCGAGCAGCC |
| | ACCTCGAGATCAAACTCTGACGTCATCAGATGAAGAGGACATTTACGCCCAC |
| | GGGCTGCCCTCTTCTTCCTCAGAGACATCCGTTACAGAGTTGGGTCCGTCTTG |
| | CTCACAACAAGATTTGTCAAGACTTGGTGCCGAAGATGCTGGTCTCCTGAAA |
| | CCTGATCAGTTTGCAGAGTCCTGGATGGGCTACTCTGGCCCGGGCTATGGGA |
| | TACTCTCCCTGGTCGTCTCCGAAAAGTATATTTGGTGTCTTGATTATAAGGGG |
| | GGTCTCTTCTGTTCTGCATTGCCGGGAGCAGGCTTGAGATGGCAAAAATTCG |
| | AGGATGCAGTACAACAAGTAGCCGTATCACCTAGTGGAGCATTGCTGTGGAA |
| | GATTGAGCAAAAATCAAATCGGGCGTTTGCTTGTGGAAAGGTAACGATTAAA |
| | GGTAAGCGACACTGGTACGAAGCACTTCCCCAGGCCGTATTTGTTGCGCTGT |
| | CCGATGACACCGCCTGGATCATTCGAACATCTGGGGATTTGTATCTTCAAACG |
| | GGGCTCAGCGTAGATAGACCTTGTGCTCGGGCGGTAAAAGTGGACTGCCCAT |
| | ACCCCTTGTCACAGATTACTGCACGAAACAACGTCGTTTGGGCTCTGACGGA |
| | GCAACGAGCTCTGCTCTATAGGGAAGGTGTAAGCTCTTTCTGCCCAGAAGGG |
| | GAACAGTGGAAGTGTGATATCGTATCAGAGCGCCAAGCTCTCGAACCCGTAT |
| | GTATCACACTGGGTGATCAGCAAACGCTTTGGGCTCTTGACATACATGGAAA |
| | TCTTTGGTTTCGCACAGGCATCATTTCAAAAAAACCACAGGGAGACGACGAC |
| | CACTGGTGGCAAGTTTCTATCACGGATTATGTCGTATTCGATCAGTGTTCACT |
| | TTTCCAGACCATAATCCATGCCACGCATTCCGTGGCAACCGCGGCTCAAGCTC |
| | CCGTAGAAAAGGTGGCAGATAAACTCCGCATGGCATTTTGGTCACAACAGCT |
| | TCAGTGCCAACCTTCTTTGCTTGGTGTGAATAACTCTGGAGTTTGGATTTCCA |
| | GTGGTAAAAACGAATTCCACGTTGCTAAGGGCAGCCTTATTGGTACTTATTG |
| | GAATCATGTTGTCCCTAGAGGAACAGCCTCTGCGACCAAGTGGGCCTTCGTA |
| | CTTGCATCAGCGGCGCCAACTAAGGAGGGTTCATTCCTCTGGCTTTGCCAGTC |
| | TAGTAAAGACCTCTGTAGTGTGTCCGCACAAAGCGCCCAAAGCCGACCAAGC |
| | ACCGTGCAGCTGCCGCCGGAGGCCGAGATGAGGGCGTACGCCGCATGTCAA |
| | GACGCTTTGTGGGCGCTTGATTCTCTGGGTCAGGTATTTATTAGGACTCTTTC |
| | AAAGAGCTGCCCCACGGGCATGCACTGGACGCGGCTGGATCTCTCTCAGCTT |
| | GGGGCCGTAAAGCTCACTTCTCTTGCGTGTGGCAACCAGCATATCTGGGCAT |
| | GCGATTCCAGAGGGGGAGTATACTTTCGAGTGGGCACACAACCTCTTAATCC |
| | GTCACTGATGCTTCCAGCGTGGATAATGATCGAGCCGCCGGTCCAACCCGCG |
| | GGTGTTTCACTTGTTAGTGTTCATTCATCTCCGAATGACCAAATGCTCTGGGT |
| | CCTCGACTCTAGATGGAATGTTCACGTACGGACGGGTATTACAGAGGAGATG |
| | CCTGTAGGTACGGCTTGGGAGCATGTCCCTGGCCTGCAGGCATGTCAACTGG |
| | CTCTGTCCACTAGGACGGTCTGGGCCAGGTGTCCAAATGGGGACCTTGCCAG |
| | ACGGTACGGAGTGACAGACAAGAATCCTGCAGGTGACTATTGGAAGAAAAT |
| | ACCCGGTAGTGTAAGCTGTTTCACCGTCACGGCCTCAGATGAACTGTGGGCT |
| | GTGGGACCACCTGGTTACCTCCTCCAGCGCTTGACTAAGACATTCTCCCATTC |
| | CCACGGCACCCAGAAAAGTTCACAAGCTGCTATGCCACATCCAGAAGACCTG |
| | GAAGATGAGTGGGAAGTTATTTGACCTTAACTTGTTTATTGCAGCTTATAATG |
| | GTTACAAATAAAGCAATAGCATCACAAATTTCACAAATAAAGCATTTTTTTC |
| | ACTGCATTCTAGTTGTGGTTTGTCCAAACTCATCAATGTATCTTATCATGTCC |
| | ATCGATAAGGATCTAGGAACCCCTAGTGATGGGCGCGCCATCGGATCCCGGG |
| | CCCGTCGACGCGCCGGCGTCTAGAAGGAACCCCTAGTGATGGAGTTGGCCAC |
| | TCCCTCTCTGCGCGCTCGCTCGCTCACTGAGGCCGGGCGACCAAAGGTCGCC |
| | CGACGCCCGGGCTTTGCCCGGGCGGCCTCAGTGAGCGAGCGAGCGCGCAGA |
| | GAGGGAGTGGCCAACAGCTGCATTAATGAATCCTGCAGGCGGCCAACGCGC |
| | GGGGAGAGGCGGTTTGCGTATTGGGCGCTCTTCCGCTTCCTCGCTCACTGACT |
| | CGCTGCGCTCGGTCGTTCGGCTGCGGCGAGCGGTATCAGCTCACTCAAAGGC |
| | GGTAATACGGTTATCCACAGAATCAGGGGATAACGCAGGAAAGAACATGTG |
| | AGCAAAAGGCCAGCAAAAGGCCAGGAACCGTAAAAAGGCCGCGTTGCTGGC |
| | GTTTTTCCATAGGCTCCGCCCCCCTGACGAGCATCACAAAAATCGACGCTCA |
| | AGTCAGAGGTGGCGAAACCCGACAGGACTATAAAGATACCAGGCGTTTCCCC |
| | CTGGAAGCTCCCTCGTGCGCTCTCCTGTTCCGACCCTGCCGCTTACCGGATAC |
| | CTGTCCGCCTTTCTCCCTTCGGGAAGCGTGGCGCTTTCTCATAGCTCACGCTG |
| | TAGGTATCTCAGTTCGGTGTAGGTCGTTCGCTCCAAGCTGGGCTGTGTGCACG |
| | AACCCCCCGTTCAGCCCGACCGCTGCGCCTTATCCGGTAACTATCGTCTTGAG |
| | TCCAACCCGGTAAGACACGACTTATCGCCACTGGCAGCAGCCACTGGTAACA |
| | GGATTAGCAGAGCGAGGTATGTAGGCGGTGCTACAGAGTTCTTGAAGTGGTG |
| | GCCTAACTACGGCTACACTAGAAGAACAGTATTTGGTATCTGCGCTCTGCTG |
| | AAGCCAGTTACCTTCGGAAAAAGAGTTGGTAGCTCTTGATCCGGCAAACAAA |
| | CCACCGCTGGTAGCGGTGGTTTTTTTGTTTGCAAGCAGCAGATTACGCGCAGA |
| | AAAAAAGGATCTCAAGAAGATCCTTTGATCTTTTCTACGGGGTCTGACGCTC |
| | AGTGGAACGAAAACTCACGTTAAGGGATTTTGGTCATGATTACCAATGCTTA |
| | ATCAGTGAGGCACCTATCTCAGCGATCTGTCTATTTCGTTCATCCATAGTTGC |
| | CTGACTCCCCGTCGTGTAGATAACTACGATACGGGAGGGCTTACCATCTGGC |
| | CCCAGTGCTGCAATGATACCGCGAGACCCACGCTCACCGGCTCCAGATTTAT |
| | CAGCAATAAACCAGCCAGCCGGAAGGGCCGAGCGCAGAAGTGGTCCTGCAA |
| | CTTTATCCGCCTCCATCCAGTCTATTAATTGTTGCCGGGAAGCTAGAGTAAGT |
| | AGTTCGCCAGTTAATAGTTTGCGCAACGTTGTTGCCATTGCTACAGGCATCGT |
| | GGTGTCACGCTCGTCGTTTGGTATGGCTTCATTCAGCTCCGGTTCCCAACGAT |
| | CAAGGCGAGTTACATGATCCCCCATGTTGTGCAAAAAAGCGGTTAGCTCCTT |
| | CGGTCCTCCGATCGTTGTCAGAAGTAAGTTGGCCGCAGTGTTATCACTCATGG |
| | TTATGGCAGCACTGCATAATTCTCTTACTGTCATGCCATCCGTAAGATGCTTT |
| | TCTGTGACTGGTGAGTACTCAACCAAGTCATTCTGAGAATAGTGTATGCGGC |
| | GACCGAGTTGCTCTTGCCCGGCGTCAATACGGGATAATACCGCGCCACATAG |
| | CAGAACTTTAAAAGTGCTCATCATTGGAAAACGTTCTTCGGGGCGAAAACTC |
| | TCAAGGATCTTACCGCTGTTGAGATCCAGTTCGATGTAACCCACTCGTGCACC |
| | CAACTGATCTTCAGCATCTTTTACTTTCACCAGCGTTTCTGGGTGAGCAAAAA |
| | CAGGAAGGCAAAATGCCGCAAAAAAGGGAATAAGGGCGACACGGAAATGTT |
| | GAATACTCATACTCTTCCTTTTTCAATATTATTGAAGCATTTATCAGGGTTATT |
| | GTCTCTATGAGCGGATACATATTTGAATGTATTTAGAAAAATAAACAAATAG |
| | GGGTTCCGCGTCATGAGATTATCAAAAAGGATCTTCACCTAGATCCTTTTCAC |
| | GTAGAAAGCCAGTCCGCAGAAACGGTGCTGACCCCCTGTGGAATGTGTGTCA |
| | GTTAGGGTGTGGAAAGTCCCCAGGCTCCCCAGCAGGCAGAAGTATGCAAAGC |
| | ATGCATCTCAATTAGTCAGCAACCAGGTGTGGAAAGTCCCCAGGCTCCCCAG |
| | CAGGCAGAAGTATGCAAAGCATGCATCTCAATTAGTCAGCAACCATAGTCCC |
| | GCCCCTAACTCCGCCCATCCCGCCCCTAACTCCGCCCAGTTCCGCCCATTCTC |
| | CGCCCCATGGCTGACTAATTTTTTTTATTTATGCAGAGGCCGAGGCCGCCTCG |
| | GCCTCTGAGCTATTCCAGAAGTAGTGAGGAGGCTTGGATGAATGTCAGCTAC |
| | TGGGCTATCTGGACAAGGGAAAACGCAAGCGCAAAGAGAAAGCAGGTAGCT |
| | TGCAGTGGGCTTACATGGCGATAGCTAGACTGGGCGGTTTTATGGACAGCAA |
| | GCGAACCGGAATTGCCAGCTGGGGCGCCCTCTGGTAAGGTTGGGAAGCCCTG |
| | CAAAGTAAACTGGATGGCTTTCTTGCCGCCAAGGATCTGATGGCGCAGGGGA |
| | TCAAGCTCTGATCAAGAGACAGGATGAGGATCGTTTCGCATGATTGAACAAG |
| | ATGGATTGCACGCAGGTTCTCCGGCCGCTTGGGTGGAGAGGCTATTCGGCTA |
| | TGACTGGGCACAACAGACAATCGGCTGCTCTGATGCCGCCGTGTTCCGGCTG |
| | TCAGCGCAGGGGCGCCCGGTTCTTTTTGTCAAGACCGACCTGTCCGGTGCCCT |
| | GAATGAACTGCAAGACGAGGCAGCGCGGCTATCGTGGCTGGCCACGACGGG |
| | CGTTCCTTGCGCAGCTGTGCTCGACGTTGTCACTGAAGCGGGAAGGGACTGG |
| | CTGCTATTGGGCGAAGTGCCGGGGCAGGATCTCCTGTCATCTCACCTTGCTCC |
| | TGCCGAGAAAGTATCCATCATGGCTGATGCAATGCGGCGGCTGCATACGCTT |
| | GATCCGGCTACCTGCCCATTCGACCACCAAGCGAAACATCGCATCGAGCGAG |
| | CACGTACTCGGATGGAAGCCGGTCTTGTCGATCAGGATGATCTGGACGAAGA |
| | GCATCAGGGGCTCGCGCCAGCCGAACTGTTCGCCAGGCTCAAGGCGAGCATG |
| | CCCGACGGCGAGGATCTCGTCGTGACCCATGGCGATGCCTGCTTGCCGAATA |
| | TCATGGTGGAAAATGGCCGCTTTTCTGGATTCATCGACTGTGGCCGGCTGGGT |
| | GTGGCGGACCGCTATCAGGACATAGCGTTGGCTACCCGTGATATTGCTGAAG |
| | AGCTTGGCGGCGAATGGGCTGACCGCTTCCTCGTGCTTTACGGTATCGCCGCT |
| | CCCGATTCGCAGCGCATCGCCTTCTATCGCCTTCTTGACGAGTTCTTCTGAAT |
| | TTTCACACAAAAAACCAACACACAGATGTAATGAAAATAAAGATATTTTATT |
| | GTTAAAATTTTTGTTAAATCAGCTCATTTTTTAACCAATAGGCCGAAATCGGC |
| | AAAATCCCTTATAAATCAAAAGAATAGACCGAGATAGGGTTGAGTGTTGTTC |
| | CAGTTTGGAACAAGAGTCCACTATTAAAGAACGTGGACTCCAACGTCAAAGG |
| | GCGAAAAACCGTCTATCAGGGCGATGGCCCACTACGTGAACCATCACCCTAA |
| | TCAAGTTTTTTGGGGTCGAGGTGCCGTAAAGCACTAAATCGGAACCCTAAAG |
| | GGAGCCCCCGATTTAGAGCTTGACGGGGAAAGCCGGCGAACGTGGCGAGAA |
| | AGGAAGGGAAGAAAGCGAAAGGAGCGGGCGCTAGGGCGCTGGCAAGTGTA |
| | GCGGTCACGCTGCGCGTAACCACCACACCCGCCGCGCTTAATGCGCCGCTAC |
| | AGGGCGCGTCGCGTCCATTCGCCATTCAGGCTGCGCAACTGTTGGGAAGGGC |
| | TGCAG |

TABLE 6
Nucleotide Sequence Information for TECPR2 Construct 5

| SEQ ID NO: | Nucleotide Sequence (5′→3′) |
|---|---|
| 5 | TTAATTAACTGCAGACGCCAGCTGTTGGCCACTCCCTCTCTGCGCGCTCGCTC |
| | GCTCACTGAGGCCGGGCGACCAAAGGTCGCCCGACGCCCGGGCTTTGCCCGG |
| | GCGGCCTCAGTGAGCGAGCGAGCGCGCAGAGAGGGAGTGGCCAACTCCATC |
| | ACTAGGGGTTCCTTCTAGAGGCGCGCCAAGCTTGGTACCTTCGCATATTAAG |
| | GTGACGCGTGTGGCCTCGAACACCGAGCGACCCTGCAGCGACCCGCTTAAAA |
| | GAGTCGACAAGAGGTAAGGGTTTAAGGGATGGTTGGTTGGTGGGGTATTAAT |
| | GTTTAATTACCTGTTTTACAGGCCTGAAATCACTTGGTTTTAGGTTGGGAAGC |
| | TTGATATCCCTGCAGGGCGGCCGCCGCATGGCGAGCATCTCAGAGCCGGTCA |
| | CGTTTAGAGAGTTCTGTCCACTTTATTACCTTCTTAATGCAATTCCAACTAAA |
| | ATCCAGAAAGGATTTCGATCTATAGTAGTTTATTTGACCGCATTGGATACGAA |
| | CGGCGACTACATCGCGGTTGGCTCAAGCATCGGAATGCTCTATTTGTACTGTC |
| | GGCACCTCAATCAGATGAGGAAATATAACTTCGAAGGCAAGACCGAATCCAT |
| | TACAGTTGTGAAGCTCCTCTCCTGTTTCGACGACCTGGTGGCCGCCGGCACCG |
| | CCTCCGGCCGAGTTGCAGTGTTCCAACTTGTATCAAGCCTCCCCGGCAGGAA |
| | CAAGCAACTGAGAAGATTTGACGTTACGGGTATTCATAAGAATTCCATAACG |
| | GCTCTGGCATGGAGCCCGAACGGGATGAAACTGTTCAGCGGTGACGATAAAG |
| | GGAAGATCGTGTACTCTTCTCTGGATCTGGACCAAGGGCTTTGCAACTCCCAA |
| | CTTGTTCTCGAGGAACCATCTTCTATCGTCCAGCTTGACTACAGTCAGAAGGT |
| | CTTGCTTGTGTCAACCCTTCAGCGAAGCCTTCTTTTTTATACGGAAGAGAAGT |
| | CCGTTCGGCAGATTGGGACGCAACCCCGCAAAAGCACCGGTAAGTTTGGCGC |
| | ATGTTTTATCCCAGGACTGTGTAAACAATCTGACTTGACACTTTATGCCTCAC |
| | GCCCAGGTCTGAGACTCTGGAAAGCCGACGTACACGGCACTGTACAAGCTAC |
| | ATTTATCTTGAAGGACGCTTTCGCCGGCGGCGTTAAGCCCTTCGAACTGCATC |
| | CCAGATTGGAGTCCCCCAATAGCGGTTCCTGTTCCCTTCCAGAGAGGCATCTG |
| | GGTCTTGTTTCCTGTTTCTTTCAGGAAGGCTGGGTGCTCAGCTGGAACGAATA |
| | CTCAATATACCTGCTTGATACTGTGAACCAAGCTACTGTTGCGGGGCTGGAG |
| | GGAAGCGGTGATATTGTCAGCGTGTCATGCACTGAAAATGAGATTTTTTTTCT |
| | CAAAGGGGATCGAAATATAATTCGGATCTCATCCCGGCCCGAAGGACTTACT |
| | AGCACTGTAAGAGACGGACTGGAGATGAGCGGATGTTCCGAACGCGTGCAC |
| | GTACAACAGGCTGAGAAGCTGCCCGGAGCTACTGTGAGTGAAACACGGTTGC |
| | GGGGCTCATCCATGGCTTCTTCAGTTGCAAGCGAACCGCGATCACGGTCATC |
| | ATCATTGAACTCAACTGATAGCGGCAGTGGACTGCTTCCCCCGGGATTGCAG |
| | GCTACCCCCGAACTCGGTAAAGGTTCTCAACCTCTCTCTCAGCGCTTCAACGC |
| | GATCTCCTCAGAAGATTTCGACCAGGAGTTGGTTGTTAAGCCCATCAAAGTC |
| | AAGCGCAAAAAGAAAAAAAAAAAGACCGAAGGGGGCTCCCGGAGTACGTGC |
| | CACTCCTCTCTTGAGAGTACTCCGTGTAGTGAGTTTCCAGGCGACAGTCCGCA |
| | GTCTCTGAACACAGACCTTTTGAGTATGACAAGCTCAGTGCTTGGGAGCAGT |
| | GTAGACCAACTTTCAGCTGAGTCACCGGATCAGGAGTCTTCTTTCAATGGCG |
| | AGGTTAATGGTGTACCACAGGAGAATACAGATCCCGAAACATTCAACGTCCT |
| | CGAAGTCTCTGGTAGTATGCCAGATTCCCTCGCTGAAGAAGATGACATACGG |
| | ACGGAAATGCCCCATTGCCACCACGCGCATGGTCGAGAGCTTCTTAACGGAG |
| | CAAGGGAAGACGTAGGGGGCTCCGACGTAACGGGGTTGGGTGATGAACCGT |
| | GCCCGGCTGACGACGGTCCCAATAGTACACAGTTGCCGTTCCAAGAACAGGA |
| | CAGTTCCCCCGGCGCGCATGATGGAGAGGACATCCAGCCGATAGGTCCACAA |
| | AGTACTTTCTGCGAGGTTCCACTTCTCAACTCACTCACTGTCCCGAGCTCTCTT |
| | TCATGGGCCCCGTCAGCAGAACAATGGTTGCCAGGAACGCGCGCTGATGAAG |
| | GCTCCCCGGTTGAACCATCCCAAGAGCAGGACATACTGACATCCATGGAAGC |
| | TAGTGGGCACCTTTCTACTAATCTGTGGCACGCGGTCACGGATGACGATACG |
| | GGTCAGAAGGAGATACCAATCTCCGAGCGCGTACTCGGCTCCGTAGGCGGAC |
| | AACTTACTCCCGTATCTGCGCTCGCAGCCAGTACCCACAAGCCATGGCTCGA |
| | GCAGCCACCTCGAGATCAAACTCTGACGTCATCAGATGAAGAGGACATTTAC |
| | GCCCACGGGCTGCCCTCTTCTTCCTCAGAGACATCCGTTACAGAGTTGGGTCC |
| | GTCTTGCTCACAACAAGATTTGTCAAGACTTGGTGCCGAAGATGCTGGTCTCC |
| | TGAAACCTGATCAGTTTGCAGAGTCCTGGATGGGCTACTCTGGCCCGGGCTA |
| | TGGGATACTCTCCCTGGTCGTCTCCGAAAAGTATATTTGGTGTCTTGATTATA |
| | AGGGGGGTCTCTTCTGTTCTGCATTGCCGGGAGCAGGCTTGAGATGGCAAAA |
| | ATTCGAGGATGCAGTACAACAAGTAGCCGTATCACCTAGTGGAGCATTGCTG |
| | TGGAAGATTGAGCAAAAATCAAATCGGGCGTTTGCTTGTGGAAAGGTAACGA |
| | TTAAAGGTAAGCGACACTGGTACGAAGCACTTCCCCAGGCCGTATTTGTTGC |
| | GCTGTCCGATGACACCGCCTGGATCATTCGAACATCTGGGGATTTGTATCTTC |
| | AAACGGGGCTCAGCGTAGATAGACCTTGTGCTCGGGCGGTAAAAGTGGACTG |
| | CCCATACCCCTTGTCACAGATTACTGCACGAAACAACGTCGTTTGGGCTCTGA |
| | CGGAGCAACGAGCTCTGCTCTATAGGGAAGGTGTAAGCTCTTTCTGCCCAGA |
| | AGGGGAACAGTGGAAGTGTGATATCGTATCAGAGCGCCAAGCTCTCGAACCC |
| | GTATGTATCACACTGGGTGATCAGCAAACGCTTTGGGCTCTTGACATACATG |
| | GAAATCTTTGGTTTCGCACAGGCATCATTTCAAAAAAACCACAGGGAGACGA |
| | CGACCACTGGTGGCAAGTTTCTATCACGGATTATGTCGTATTCGATCAGTGTT |
| | CACTTTTCCAGACCATAATCCATGCCACGCATTCCGTGGCAACCGCGGCTCAA |
| | GCTCCCGTAGAAAAGGTGGCAGATAAACTCCGCATGGCATTTTGGTCACAAC |
| | AGCTTCAGTGCCAACCTTCTTTGCTTGGTGTGAATAACTCTGGAGTTTGGATT |
| | TCCAGTGGTAAAAACGAATTCCACGTTGCTAAGGGCAGCCTTATTGGTACTT |
| | ATTGGAATCATGTTGTCCCTAGAGGAACAGCCTCTGCGACCAAGTGGGCCTT |
| | CGTACTTGCATCAGCGGCGCCAACTAAGGAGGGTTCATTCCTCTGGCTTTGCC |
| | AGTCTAGTAAAGACCTCTGTAGTGTGTCCGCACAAAGCGCCCAAAGCCGACC |
| | AAGCACCGTGCAGCTGCCGCCGGAGGCCGAGATGAGGGCGTACGCCGCATG |
| | TCAAGACGCTTTGTGGGCGCTTGATTCTCTGGGTCAGGTATTTATTAGGACTC |
| | TTTCAAAGAGCTGCCCCACGGGCATGCACTGGACGCGGCTGGATCTCTCTCA |
| | GCTTGGGGCCGTAAAGCTCACTTCTCTTGCGTGTGGCAACCAGCATATCTGGG |
| | CATGCGATTCCAGAGGGGGAGTATACTTTCGAGTGGGCACACAACCTCTTAA |
| | TCCGTCACTGATGCTTCCAGCGTGGATAATGATCGAGCCGCCGGTCCAACCC |
| | GCGGGTGTTTCACTTGTTAGTGTTCATTCATCTCCGAATGACCAAATGCTCTG |
| | GGTCCTCGACTCTAGATGGAATGTTCACGTACGGACGGGTATTACAGAGGAG |
| | ATGCCTGTAGGTACGGCTTGGGAGCATGTCCCTGGCCTGCAGGCATGTCAAC |
| | TGGCTCTGTCCACTAGGACGGTCTGGGCCAGGTGTCCAAATGGGGACCTTGC |
| | CAGACGGTACGGAGTGACAGACAAGAATCCTGCAGGTGACTATTGGAAGAA |
| | AATACCCGGTAGTGTAAGCTGTTTCACCGTCACGGCCTCAGATGAACTGTGG |
| | GCTGTGGGACCACCTGGTTACCTCCTCCAGCGCTTGACTAAGACATTCTCCCA |
| | TTCCCACGGCACCCAGAAAAGTTCACAAGCTGCTATGCCACATCCAGAAGAC |
| | CTGGAAGATGAGTGGGAAGTTATTTGACCTTAACTTGTTTATTGCAGCTTATA |
| | ATGGTTACAAATAAAGCAATAGCATCACAAATTTCACAAATAAAGCATTTTT |
| | TTCACTGCATTCTAGTTGTGGTTTGTCCAAACTCATCAATGTATCTTATCATGT |
| | CCATCGATAAGGATCTAGGAACCCCTAGTGATGGGCGCGCCATCGGATCCCG |
| | GGCCCGTCGACGCGCCGGCGTCTAGAAGGAACCCCTAGTGATGGAGTTGGCC |
| | ACTCCCTCTCTGCGCGCTCGCTCGCTCACTGAGGCCGGGCGACCAAAGGTCG |
| | CCCGACGCCCGGGCTTTGCCCGGGCGGCCTCAGTGAGCGAGCGAGCGCGCAG |
| | AGAGGGAGTGGCCAACAGCTGCATTAATGAATCCTGCAGGCGGCCAACGCG |
| | CGGGGAGAGGCGGTTTGCGTATTGGGCGCTCTTCCGCTTCCTCGCTCACTGAC |
| | TCGCTGCGCTCGGTCGTTCGGCTGCGGCGAGCGGTATCAGCTCACTCAAAGG |
| | CGGTAATACGGTTATCCACAGAATCAGGGGATAACGCAGGAAAGAACATGT |
| | GAGCAAAAGGCCAGCAAAAGGCCAGGAACCGTAAAAAGGCCGCGTTGCTGG |
| | CGTTTTTCCATAGGCTCCGCCCCCCTGACGAGCATCACAAAAATCGACGCTCA |
| | AGTCAGAGGTGGCGAAACCCGACAGGACTATAAAGATACCAGGCGTTTCCCC |
| | CTGGAAGCTCCCTCGTGCGCTCTCCTGTTCCGACCCTGCCGCTTACCGGATAC |
| | CTGTCCGCCTTTCTCCCTTCGGGAAGCGTGGCGCTTTCTCATAGCTCACGCTG |
| | TAGGTATCTCAGTTCGGTGTAGGTCGTTCGCTCCAAGCTGGGCTGTGTGCACG |
| | AACCCCCCGTTCAGCCCGACCGCTGCGCCTTATCCGGTAACTATCGTCTTGAG |
| | TCCAACCCGGTAAGACACGACTTATCGCCACTGGCAGCAGCCACTGGTAACA |
| | GGATTAGCAGAGCGAGGTATGTAGGCGGTGCTACAGAGTTCTTGAAGTGGTG |
| | GCCTAACTACGGCTACACTAGAAGAACAGTATTTGGTATCTGCGCTCTGCTG |
| | AAGCCAGTTACCTTCGGAAAAAGAGTTGGTAGCTCTTGATCCGGCAAACAAA |
| | CCACCGCTGGTAGCGGTGGTTTTTTTGTTTGCAAGCAGCAGATTACGCGCAGA |
| | AAAAAAGGATCTCAAGAAGATCCTTTGATCTTTTCTACGGGGTCTGACGCTC |
| | AGTGGAACGAAAACTCACGTTAAGGGATTTTGGTCATGATTACCAATGCTTA |
| | ATCAGTGAGGCACCTATCTCAGCGATCTGTCTATTTCGTTCATCCATAGTTGC |
| | CTGACTCCCCGTCGTGTAGATAACTACGATACGGGAGGGCTTACCATCTGGC |
| | CCCAGTGCTGCAATGATACCGCGAGACCCACGCTCACCGGCTCCAGATTTAT |
| | CAGCAATAAACCAGCCAGCCGGAAGGGCCGAGCGCAGAAGTGGTCCTGCAA |
| | CTTTATCCGCCTCCATCCAGTCTATTAATTGTTGCCGGGAAGCTAGAGTAAGT |
| | AGTTCGCCAGTTAATAGTTTGCGCAACGTTGTTGCCATTGCTACAGGCATCGT |
| | GGTGTCACGCTCGTCGTTTGGTATGGCTTCATTCAGCTCCGGTTCCCAACGAT |
| | CAAGGCGAGTTACATGATCCCCCATGTTGTGCAAAAAAGCGGTTAGCTCCTT |
| | CGGTCCTCCGATCGTTGTCAGAAGTAAGTTGGCCGCAGTGTTATCACTCATGG |
| | TTATGGCAGCACTGCATAATTCTCTTACTGTCATGCCATCCGTAAGATGCTTT |
| | TCTGTGACTGGTGAGTACTCAACCAAGTCATTCTGAGAATAGTGTATGCGGC |
| | GACCGAGTTGCTCTTGCCCGGCGTCAATACGGGATAATACCGCGCCACATAG |
| | CAGAACTTTAAAAGTGCTCATCATTGGAAAACGTTCTTCGGGGCGAAAACTC |
| | TCAAGGATCTTACCGCTGTTGAGATCCAGTTCGATGTAACCCACTCGTGCACC |
| | CAACTGATCTTCAGCATCTTTTACTTTCACCAGCGTTTCTGGGTGAGCAAAAA |
| | CAGGAAGGCAAAATGCCGCAAAAAAGGGAATAAGGGCGACACGGAAATGTT |
| | GAATACTCATACTCTTCCTTTTTCAATATTATTGAAGCATTTATCAGGGTTATT |
| | GTCTCTATGAGCGGATACATATTTGAATGTATTTAGAAAAATAAACAAATAG |
| | GGGTTCCGCGTCATGAGATTATCAAAAAGGATCTTCACCTAGATCCTTTTCAC |
| | GTAGAAAGCCAGTCCGCAGAAACGGTGCTGACCCCCTGTGGAATGTGTGTCA |
| | GTTAGGGTGTGGAAAGTCCCCAGGCTCCCCAGCAGGCAGAAGTATGCAAAGC |
| | ATGCATCTCAATTAGTCAGCAACCAGGTGTGGAAAGTCCCCAGGCTCCCCAG |
| | CAGGCAGAAGTATGCAAAGCATGCATCTCAATTAGTCAGCAACCATAGTCCC |
| | GCCCCTAACTCCGCCCATCCCGCCCCTAACTCCGCCCAGTTCCGCCCATTCTC |
| | CGCCCCATGGCTGACTAATTTTTTTTATTTATGCAGAGGCCGAGGCCGCCTCG |
| | GCCTCTGAGCTATTCCAGAAGTAGTGAGGAGGCTTGGATGAATGTCAGCTAC |
| | TGGGCTATCTGGACAAGGGAAAACGCAAGCGCAAAGAGAAAGCAGGTAGCT |
| | TGCAGTGGGCTTACATGGCGATAGCTAGACTGGGCGGTTTTATGGACAGCAA |
| | GCGAACCGGAATTGCCAGCTGGGGCGCCCTCTGGTAAGGTTGGGAAGCCCTG |
| | CAAAGTAAACTGGATGGCTTTCTTGCCGCCAAGGATCTGATGGCGCAGGGGA |
| | TCAAGCTCTGATCAAGAGACAGGATGAGGATCGTTTCGCATGATTGAACAAG |
| | ATGGATTGCACGCAGGTTCTCCGGCCGCTTGGGTGGAGAGGCTATTCGGCTA |
| | TGACTGGGCACAACAGACAATCGGCTGCTCTGATGCCGCCGTGTTCCGGCTG |
| | TCAGCGCAGGGGCGCCCGGTTCTTTTTGTCAAGACCGACCTGTCCGGTGCCCT |
| | GAATGAACTGCAAGACGAGGCAGCGCGGCTATCGTGGCTGGCCACGACGGG |
| | CGTTCCTTGCGCAGCTGTGCTCGACGTTGTCACTGAAGCGGGAAGGGACTGG |
| | CTGCTATTGGGCGAAGTGCCGGGGCAGGATCTCCTGTCATCTCACCTTGCTCC |
| | TGCCGAGAAAGTATCCATCATGGCTGATGCAATGCGGCGGCTGCATACGCTT |
| | GATCCGGCTACCTGCCCATTCGACCACCAAGCGAAACATCGCATCGAGCGAG |
| | CACGTACTCGGATGGAAGCCGGTCTTGTCGATCAGGATGATCTGGACGAAGA |
| | GCATCAGGGGCTCGCGCCAGCCGAACTGTTCGCCAGGCTCAAGGCGAGCATG |
| | CCCGACGGCGAGGATCTCGTCGTGACCCATGGCGATGCCTGCTTGCCGAATA |
| | TCATGGTGGAAAATGGCCGCTTTTCTGGATTCATCGACTGTGGCCGGCTGGGT |
| | GTGGCGGACCGCTATCAGGACATAGCGTTGGCTACCCGTGATATTGCTGAAG |
| | AGCTTGGCGGCGAATGGGCTGACCGCTTCCTCGTGCTTTACGGTATCGCCGCT |
| | CCCGATTCGCAGCGCATCGCCTTCTATCGCCTTCTTGACGAGTTCTTCTGAAT |
| | TTTCACACAAAAAACCAACACACAGATGTAATGAAAATAAAGATATTTTATT |
| | GTTAAAATTTTTGTTAAATCAGCTCATTTTTTAACCAATAGGCCGAAATCGGC |
| | AAAATCCCTTATAAATCAAAAGAATAGACCGAGATAGGGTTGAGTGTTGTTC |
| | CAGTTTGGAACAAGAGTCCACTATTAAAGAACGTGGACTCCAACGTCAAAGG |
| | GCGAAAAACCGTCTATCAGGGCGATGGCCCACTACGTGAACCATCACCCTAA |
| | TCAAGTTTTTTGGGGTCGAGGTGCCGTAAAGCACTAAATCGGAACCCTAAAG |
| | GGAGCCCCCGATTTAGAGCTTGACGGGGAAAGCCGGCGAACGTGGCGAGAA |
| | AGGAAGGGAAGAAAGCGAAAGGAGCGGGCGCTAGGGCGCTGGCAAGTGTA |
| | GCGGTCACGCTGCGCGTAACCACCACACCCGCCGCGCTTAATGCGCCGCTAC |
| | AGGGCGCGTCGCGTCCATTCGCCATTCAGGCTGCGCAACTGTTGGGAAGGGC |
| | TGCAG |

TABLE 7
Nucleotide Sequence Information for TECPR2 Construct 6

| SEQ ID NO: | Nucleotide Sequence (5′→3′) |
|---|---|
| 18 | TTGGCCACTCCCTCTCTGCGCGCTCGCTCGCTCACTGAGGCCGGGCGACCAAA |
| | GGTCGCCCGACGCCCGGGCTTTGCCCGGGCGGCCTCAGTGAGCGAGCGAGCG |
| | CGCAGAGAGGGAGTGGCCAACTCCATCACTAGGGGTTCCTTCTAGAGGCGCG |
| | CCAAGCTTGGTACCCCATGGAGGCGGTACTATGTAGATGAGAATTCAGGTGC |
| | AAACTGGGAAAAGCAACTGCTTCCAAATATTTGTGATTTTTACAGTGTAGTTT |
| | TGGAAAAACTCTTAGCCTACCAATTCTTCTAAGTGTTTTAAAATGTGGGAGCC |
| | AGTACACATGAAGTTATAGAGTGTTTTAATGAGGCTTAAATATTTACCGTAAC |
| | TATGAAATGCTACGCATATCATGCTGTTCAGGCTCCGTGGCCACGCAACTCAT |
| | ACTCCGCGGCCGCACTAGTGCCGCCATGGCGAGCATCTCAGAGCCGGTCACG |
| | TTTAGAGAGTTCTGTCCACTTTATTACCTTCTTAATGCAATTCCAACTAAAAT |
| | CCAGAAAGGATTTCGATCTATAGTAGTTTATTTGACCGCATTGGATACGAAC |
| | GGCGACTACATCGCGGTTGGCTCAAGCATCGGAATGCTCTATTTGTACTGTCG |
| | GCACCTCAATCAGATGAGGAAATATAACTTCGAAGGCAAGACCGAATCCATT |
| | ACAGTTGTGAAGCTCCTCTCCTGTTTCGACGACCTGGTGGCCGCCGGCACCGC |
| | CTCCGGCCGAGTTGCAGTGTTCCAACTTGTATCAAGCCTCCCCGGCAGGAAC |
| | AAGCAACTGAGAAGATTTGACGTTACGGGTATTCATAAGAATTCCATAACGG |
| | CTCTGGCATGGAGCCCGAACGGGATGAAACTGTTCAGCGGTGACGATAAAGG |
| | GAAGATCGTGTACTCTTCTCTGGATCTGGACCAAGGGCTTTGCAACTCCCAAC |
| | TTGTTCTCGAGGAACCATCTTCTATCGTCCAGCTTGACTACAGTCAGAAGGTC |
| | TTGCTTGTGTCAACCCTTCAGCGAAGCCTTCTTTTTTATACGGAAGAGAAGTC |
| | CGTTCGGCAGATTGGGACGCAACCCCGCAAAAGCACCGGTAAGTTTGGCGCA |
| | TGTTTTATCCCAGGACTGTGTAAACAATCTGACTTGACACTTTATGCCTCACG |
| | CCCAGGTCTGAGACTCTGGAAAGCCGACGTACACGGCACTGTACAAGCTACA |
| | TTTATCTTGAAGGACGCTTTCGCCGGCGGCGTTAAGCCCTTCGAACTGCATCC |
| | CAGATTGGAGTCCCCCAATAGCGGTTCCTGTTCCCTTCCAGAGAGGCATCTGG |
| | GTCTTGTTTCCTGTTTCTTTCAGGAAGGCTGGGTGCTCAGCTGGAACGAATAC |
| | TCAATATACCTGCTTGATACTGTGAACCAAGCTACTGTTGCGGGGCTGGAGG |
| | GAAGCGGTGATATTGTCAGCGTGTCATGCACTGAAAATGAGATTTTTTTTCTC |
| | AAAGGGGATCGAAATATAATTCGGATCTCATCCCGGCCCGAAGGACTTACTA |
| | GCACTGTAAGAGACGGACTGGAGATGAGCGGATGTTCCGAACGCGTGCACGT |
| | ACAACAGGCTGAGAAGCTGCCCGGAGCTACTGTGAGTGAAACACGGTTGCG |
| | GGGCTCATCCATGGCTTCTTCAGTTGCAAGCGAACCGCGATCACGGTCATCAT |
| | CATTGAACTCAACTGATAGCGGCAGTGGACTGCTTCCCCCGGGATTGCAGGC |
| | TACCCCCGAACTCGGTAAAGGTTCTCAACCTCTCTCTCAGCGCTTCAACGCGA |
| | TCTCCTCAGAAGATTTCGACCAGGAGTTGGTTGTTAAGCCCATCAAAGTCAA |
| | GCGCAAAAAGAAAAAAAAAAAGACCGAAGGGGGCTCCCGGAGTACGTGCCA |
| | CTCCTCTCTTGAGAGTACTCCGTGTAGTGAGTTTCCAGGCGACAGTCCGCAGT |
| | CTCTGAACACAGACCTTTTGAGTATGACAAGCTCAGTGCTTGGGAGCAGTGT |
| | AGACCAACTTTCAGCTGAGTCACCGGATCAGGAGTCTTCTTTCAATGGCGAG |
| | GTTAATGGTGTACCACAGGAGAATACAGATCCCGAAACATTCAACGTCCTCG |
| | AAGTCTCTGGTAGTATGCCAGATTCCCTCGCTGAAGAAGATGACATACGGAC |
| | GGAAATGCCCCATTGCCACCACGCGCATGGTCGAGAGCTTCTTAACGGAGCA |
| | AGGGAAGACGTAGGGGGCTCCGACGTAACGGGGTTGGGTGATGAACCGTGC |
| | CCGGCTGACGACGGTCCCAATAGTACACAGTTGCCGTTCCAAGAACAGGACA |
| | GTTCCCCCGGCGCGCATGATGGAGAGGACATCCAGCCGATAGGTCCACAAAG |
| | TACTTTCTGCGAGGTTCCACTTCTCAACTCACTCACTGTCCCGAGCTCTCTTTC |
| | ATGGGCCCCGTCAGCAGAACAATGGTTGCCAGGAACGCGCGCTGATGAAGG |
| | CTCCCCGGTTGAACCATCCCAAGAGCAGGACATACTGACATCCATGGAAGCT |
| | AGTGGGCACCTTTCTACTAATCTGTGGCACGCGGTCACGGATGACGATACGG |
| | GTCAGAAGGAGATACCAATCTCCGAGCGCGTACTCGGCTCCGTAGGCGGACA |
| | ACTTACTCCCGTATCTGCGCTCGCAGCCAGTACCCACAAGCCATGGCTCGAG |
| | CAGCCACCTCGAGATCAAACTCTGACGTCATCAGATGAAGAGGACATTTACG |
| | CCCACGGGCTGCCCTCTTCTTCCTCAGAGACATCCGTTACAGAGTTGGGTCCG |
| | TCTTGCTCACAACAAGATTTGTCAAGACTTGGTGCCGAAGATGCTGGTCTCCT |
| | GAAACCTGATCAGTTTGCAGAGTCCTGGATGGGCTACTCTGGCCCGGGCTAT |
| | GGGATACTCTCCCTGGTCGTCTCCGAAAAGTATATTTGGTGTCTTGATTATAA |
| | GGGGGGTCTCTTCTGTTCTGCATTGCCGGGAGCAGGCTTGAGATGGCAAAAA |
| | TTCGAGGATGCAGTACAACAAGTAGCCGTATCACCTAGTGGAGCATTGCTGT |
| | GGAAGATTGAGCAAAAATCAAATCGGGCGTTTGCTTGTGGAAAGGTAACGAT |
| | TAAAGGTAAGCGACACTGGTACGAAGCACTTCCCCAGGCCGTATTTGTTGCG |
| | CTGTCCGATGACACCGCCTGGATCATTCGAACATCTGGGGATTTGTATCTTCA |
| | AACGGGGCTCAGCGTAGATAGACCTTGTGCTCGGGCGGTAAAAGTGGACTGC |
| | CCATACCCCTTGTCACAGATTACTGCACGAAACAACGTCGTTTGGGCTCTGAC |
| | GGAGCAACGAGCTCTGCTCTATAGGGAAGGTGTAAGCTCTTTCTGCCCAGAA |
| | GGGGAACAGTGGAAGTGTGATATCGTATCAGAGCGCCAAGCTCTCGAACCCG |
| | TATGTATCACACTGGGTGATCAGCAAACGCTTTGGGCTCTTGACATACATGG |
| | AAATCTTTGGTTTCGCACAGGCATCATTTCAAAAAAACCACAGGGAGACGAC |
| | GACCACTGGTGGCAAGTTTCTATCACGGATTATGTCGTATTCGATCAGTGTTC |
| | ACTTTTCCAGACCATAATCCATGCCACGCATTCCGTGGCAACCGCGGCTCAA |
| | GCTCCCGTAGAAAAGGTGGCAGATAAACTCCGCATGGCATTTTGGTCACAAC |
| | AGCTTCAGTGCCAACCTTCTTTGCTTGGTGTGAATAACTCTGGAGTTTGGATT |
| | TCCAGTGGTAAAAACGAATTCCACGTTGCTAAGGGCAGCCTTATTGGTACTT |
| | ATTGGAATCATGTTGTCCCTAGAGGAACAGCCTCTGCGACCAAGTGGGCCTT |
| | CGTACTTGCATCAGCGGCGCCAACTAAGGAGGGTTCATTCCTCTGGCTTTGCC |
| | AGTCTAGTAAAGACCTCTGTAGTGTGTCCGCACAAAGCGCCCAAAGCCGACC |
| | AAGCACCGTGCAGCTGCCGCCGGAGGCCGAGATGAGGGCGTACGCCGCATG |
| | TCAAGACGCTTTGTGGGCGCTTGATTCTCTGGGTCAGGTATTTATTAGGACTC |
| | TTTCAAAGAGCTGCCCCACGGGCATGCACTGGACGCGGCTGGATCTCTCTCA |
| | GCTTGGGGCCGTAAAGCTCACTTCTCTTGCGTGTGGCAACCAGCATATCTGGG |
| | CATGCGATTCCAGAGGGGGAGTATACTTTCGAGTGGGCACACAACCTCTTAA |
| | TCCGTCACTGATGCTTCCAGCGTGGATAATGATCGAGCCGCCGGTCCAACCC |
| | GCGGGTGTTTCACTTGTTAGTGTTCATTCATCTCCGAATGACCAAATGCTCTG |
| | GGTCCTCGACTCTAGATGGAATGTTCACGTACGGACGGGTATTACAGAGGAG |
| | ATGCCTGTAGGTACGGCTTGGGAGCATGTCCCTGGCCTGCAGGCATGTCAAC |
| | TGGCTCTGTCCACTAGGACGGTCTGGGCCAGGTGTCCAAATGGGGACCTTGC |
| | CAGACGGTACGGAGTGACAGACAAGAATCCTGCAGGTGACTATTGGAAGAA |
| | AATACCCGGTAGTGTAAGCTGTTTCACCGTCACGGCCTCAGATGAACTGTGG |
| | GCTGTGGGACCACCTGGTTACCTCCTCCAGCGCTTGACTAAGACATTCTCCCA |
| | TTCCCACGGCACCCAGAAAAGTTCACAAGCTGCTATGCCACATCCAGAAGAC |
| | CTGGAAGATGAGTGGGAAGTTATTTGATAATAGAGATCTGCTAGCTAATAAA |
| | AGATCCTTATTTTCATTGGATCTGTGTGTTGGTTTTTTGTGTGGGATCCCGGGC |
| | CCGTCGACGCGCCGGCGTCTAGAAGGAACCCCTAGTGATGGAGTTGGCCACT |
| | CCCTCTCTGCGCGCTCGCTCGCTCACTGAGGCCGGGCGACCAAAGGTCGCCC |
| | GACGCCCGGGCTTTGCCCGGGCGGCCTCAGTGAGCGAGCGAGCGCGCAGAG |
| | AGGGAGTGGCCAACAGCTGCATTAATGAATCCTGCAGGCGGCCAACGCGCG |
| | GGGAGAGGCGGTTTGCGTATTGGGCGCTCTTCCGCTTCCTCGCTCACTGACTC |
| | GCTGCGCTCGGTCGTTCGGCTGCGGCGAGCGGTATCAGCTCACTCAAAGGCG |
| | GTAATACGGTTATCCACAGAATCAGGGGATAACGCAGGAAAGAACATGTGA |
| | GCAAAAGGCCAGCAAAAGGCCAGGAACCGTAAAAAGGCCGCGTTGCTGGCG |
| | TTTTTCCATAGGCTCCGCCCCCCTGACGAGCATCACAAAAATCGACGCTCAA |
| | GTCAGAGGTGGCGAAACCCGACAGGACTATAAAGATACCAGGCGTTTCCCCC |
| | TGGAAGCTCCCTCGTGCGCTCTCCTGTTCCGACCCTGCCGCTTACCGGATACC |
| | TGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGA |
| | ACCCCCCGTTCAGCCCGACCGCTGCGCCTTATCCGGTAACTATCGTCTTGAGT |
| | CCAACCCGGTAAGACACGACTTATCGCCACTGGCAGCAGCCACTGGTAACAG |
| | GATTAGCAGAGCGAGGTATGTAGGCGGTGCTACAGAGTTCTTGAAGTGGTGG |
| | CCTAACTACGGCTACACTAGAAGAACAGTATTTGGTATCTGCGCTCTGCTGA |
| | AGCCAGTTACCTTCGGAAAAAGAGTTGGTAGCTCTTGATCCGGCAAACAAAC |
| | CACCGCTGGTAGCGGTGGTTTTTTTGTTTGCAAGCAGCAGATTACGCGCAGA |
| | AAAAAAGGATCTCAAGAAGATCCTTTGATCTTTTCTACGGGGTCTGACGCTC |
| | AGTGGAACGAAAACTCACGTTAAGGGATTTTGGTCATGATTACCAATGCTTA |
| | ATCAGTGAGGCACCTATCTCAGCGATCTGTCTATTTCGTTCATCCATAGTTGC |
| | CTGACTCCCCGTCGTGTAGATAACTACGATACGGGAGGGCTTACCATCTGGC |
| | CCCAGTGCTGCAATGATACCGCGAGACCCACGCTCACCGGCTCCAGATTTAT |
| | CAGCAATAAACCAGCCAGCCGGAAGGGCCGAGCGCAGAAGTGGTCCTGCAA |
| | CTTTATCCGCCTCCATCCAGTCTATTAATTGTTGCCGGGAAGCTAGAGTAAGT |
| | AGTTCGCCAGTTAATAGTTTGCGCAACGTTGTTGCCATTGCTACAGGCATCGT |
| | GGTGTCACGCTCGTCGTTTGGTATGGCTTCATTCAGCTCCGGTTCCCAACGAT |
| | CAAGGCGAGTTACATGATCCCCCATGTTGTGCAAAAAAGCGGTTAGCTCCTT |
| | CGGTCCTCCGATCGTTGTCAGAAGTAAGTTGGCCGCAGTGTTATCACTCATGG |
| | TTATGGCAGCACTGCATAATTCTCTTACTGTCATGCCATCCGTAAGATGCTTT |
| | TCTGTGACTGGTGAGTACTCAACCAAGTCATTCTGAGAATAGTGTATGCGGC |
| | GACCGAGTTGCTCTTGCCCGGCGTCAATACGGGATAATACCGCGCCACATAG |
| | CAGAACTTTAAAAGTGCTCATCATTGGAAAACGTTCTTCGGGGCGAAAACTC |
| | TCAAGGATCTTACCGCTGTTGAGATCCAGTTCGATGTAACCCACTCGTGCACC |
| | CAACTGATCTTCAGCATCTTTTACTTTCACCAGCGTTTCTGGGTGAGCAAAAA |
| | CAGGAAGGCAAAATGCCGCAAAAAAGGGAATAAGGGCGACACGGAAATGTT |
| | GAATACTCATACTCTTCCTTTTTCAATATTATTGAAGCATTTATCAGGGTTATT |
| | GTCTCTATGAGCGGATACATATTTGAATGTATTTAGAAAAATAAACAAATAG |
| | GGGTTCCGCGTCATGAGATTATCAAAAAGGATCTTCACCTAGATCCTTTTCAC |
| | GTAGAAAGCCAGTCCGCAGAAACGGTGCTGACCCCCTGTGGAATGTGTGTCA |
| | GTTAGGGTGTGGAAAGTCCCCAGGCTCCCCAGCAGGCAGAAGTATGCAAAGC |
| | ATGCATCTCAATTAGTCAGCAACCAGGTGTGGAAAGTCCCCAGGCTCCCCAG |
| | CAGGCAGAAGTATGCAAAGCATGCATCTCAATTAGTCAGCAACCATAGTCCC |
| | GCCCCTAACTCCGCCCATCCCGCCCCTAACTCCGCCCAGTTCCGCCCATTCTC |
| | CGCCCCATGGCTGACTAATTTTTTTTATTTATGCAGAGGCCGAGGCCGCCTCG |
| | GCCTCTGAGCTATTCCAGAAGTAGTGAGGAGGCTTGGATGAATGTCAGCTAC |
| | TGGGCTATCTGGACAAGGGAAAACGCAAGCGCAAAGAGAAAGCAGGTAGCT |
| | TGCAGTGGGCTTACATGGCGATAGCTAGACTGGGCGGTTTTATGGACAGCAA |
| | GCGAACCGGAATTGCCAGCTGGGGCGCCCTCTGGTAAGGTTGGGAAGCCCTG |
| | CAAAGTAAACTGGATGGCTTTCTTGCCGCCAAGGATCTGATGGCGCAGGGGA |
| | TCAAGCTCTGATCAAGAGACAGGATGAGGATCGTTTCGCATGATTGAACAAG |
| | ATGGATTGCACGCAGGTTCTCCGGCCGCTTGGGTGGAGAGGCTATTCGGCTA |
| | TGACTGGGCACAACAGACAATCGGCTGCTCTGATGCCGCCGTGTTCCGGCTG |
| | TCAGCGCAGGGGCGCCCGGTTCTTTTTGTCAAGACCGACCTGTCCGGTGCCCT |
| | GAATGAACTGCAAGACGAGGCAGCGCGGCTATCGTGGCTGGCCACGACGGG |
| | CGTTCCTTGCGCAGCTGTGCTCGACGTTGTCACTGAAGCGGGAAGGGACTGG |
| | CTGCTATTGGGCGAAGTGCCGGGGCAGGATCTCCTGTCATCTCACCTTGCTCC |
| | TGCCGAGAAAGTATCCATCATGGCTGATGCAATGCGGCGGCTGCATACGCTT |
| | GATCCGGCTACCTGCCCATTCGACCACCAAGCGAAACATCGCATCGAGCGAG |
| | CACGTACTCGGATGGAAGCCGGTCTTGTCGATCAGGATGATCTGGACGAAGA |
| | GCATCAGGGGCTCGCGCCAGCCGAACTGTTCGCCAGGCTCAAGGCGAGCATG |
| | CCCGACGGCGAGGATCTCGTCGTGACCCATGGCGATGCCTGCTTGCCGAATA |
| | TCATGGTGGAAAATGGCCGCTTTTCTGGATTCATCGACTGTGGCCGGCTGGGT |
| | GTGGCGGACCGCTATCAGGACATAGCGTTGGCTACCCGTGATATTGCTGAAG |
| | AGCTTGGCGGCGAATGGGCTGACCGCTTCCTCGTGCTTTACGGTATCGCCGCT |
| | CCCGATTCGCAGCGCATCGCCTTCTATCGCCTTCTTGACGAGTTCTTCTGAAT |
| | TTTCACACAAAAAACCAACACACAGATGTAATGAAAATAAAGATATTTTATT |
| | GTTAAAATTTTTGTTAAATCAGCTCATTTTTTAACCAATAGGCCGAAATCGGC |
| | AAAATCCCTTATAAATCAAAAGAATAGACCGAGATAGGGTTGAGTGTTGTTC |
| | CAGTTTGGAACAAGAGTCCACTATTAAAGAACGTGGACTCCAACGTCAAAGG |
| | GCGAAAAACCGTCTATCAGGGCGATGGCCCACTACGTGAACCATCACCCTAA |
| | TCAAGTTTTTTGGGGTCGAGGTGCCGTAAAGCACTAAATCGGAACCCTAAAG |
| | GGAGCCCCCGATTTAGAGCTTGACGGGGAAAGCCGGCGAACGTGGCGAGAA |
| | AGGAAGGGAAGAAAGCGAAAGGAGCGGGCGCTAGGGCGCTGGCAAGTGTA |
| | GCGGTCACGCTGCGCGTAACCACCACACCCGCCGCGCTTAATGCGCCGCTAC |
| | AGGGCGCGTCGCGTCCATTCGCCATTCAGGCTGCGCAACTGTTGGGAAGGGC |
| | TGCAGTTAATTAACTGCAGACGCCAGCTG |

TABLE 8
Nucleotide Sequence Information for Non-limiting Examples of TECPR2
Expression Cassettes

| | Relative 5′ to | | |
|---|---|---|---|
| | 3′ Order of | SEQ | |
| | Select | ID | |
| Construct* | Sequences** | NO: | Nucleotide Sequence (5′→3′) |
| 1 | Promoter: | 7 | CAATTGAGGGCGTCACCGCTAAGGCTCCGCCCCAGCCTGGGCTC |
| | MECP2 | | CACAACCAATGAAGGGTAATCTCGACAAAGAGCAAGGGGTGGG |
| | Chimeric | | GCGCGGGCGCGCAGGTGCAGCAGCACACAGGCTGGTCGGGAGG |
| | Intron | | GCGGGGCGCGACGTCTGCCGTGCGGGGTCCCGGCATCGGTTGCG |
| | Coding | | CGCGCGCTCCCTCCTCTCGGAGAGAGGGCTGTGGTAAAACCCGT |
| | sequence: | | CCGGAAACCACCCCTAGGACCCAAGTCGCAGCCGTTGGATCAG |
| | TECPR2 Iso1 | | GTAAGTATCAAGGTTACAAGACAGGTTTAAGGAGACCAATAGA |
| | (codon | | AACTGGGCTTGTCGAGACAGAGAAGACTCTTGCGTTTCTGATAG |
| | optimized) | | GCACCTATTGGTCTTACTGACATCCACTTTGCCTTTCTCTCCACA |
| | PolyA Signal: | | GGTGATGATATCGCGGCCGCCGCATGGCGAGCATCTCAGAGCC |
| | SV40 | | GGTCACGTTTAGAGAGTTCTGTCCACTTTATTACCTTCTTAATGC |
| | | | AATTCCAACTAAAATCCAGAAAGGATTTCGATCTATAGTAGTTT |
| | | | ATTTGACCGCATTGGATACGAACGGCGACTACATCGCGGTTGGC |
| | | | TCAAGCATCGGAATGCTCTATTTGTACTGTCGGCACCTCAATCA |
| | | | GATGAGGAAATATAACTTCGAAGGCAAGACCGAATCCATTACA |
| | | | GTTGTGAAGCTCCTCTCCTGTTTCGACGACCTGGTGGCCGCCGG |
| | | | CACCGCCTCCGGCCGAGTTGCAGTGTTCCAACTTGTATCAAGCC |
| | | | TCCCCGGCAGGAACAAGCAACTGAGAAGATTTGACGTTACGGG |
| | | | TATTCATAAGAATTCCATAACGGCTCTGGCATGGAGCCCGAACG |
| | | | GGATGAAACTGTTCAGCGGTGACGATAAAGGGAAGATCGTGTA |
| | | | CTCTTCTCTGGATCTGGACCAAGGGCTTTGCAACTCCCAACTTGT |
| | | | TCTCGAGGAACCATCTTCTATCGTCCAGCTTGACTACAGTCAGA |
| | | | AGGTCTTGCTTGTGTCAACCCTTCAGCGAAGCCTTCTTTTTTATA |
| | | | CGGAAGAGAAGTCCGTTCGGCAGATTGGGACGCAACCCCGCAA |
| | | | AAGCACCGGTAAGTTTGGCGCATGTTTTATCCCAGGACTGTGTA |
| | | | AACAATCTGACTTGACACTTTATGCCTCACGCCCAGGTCTGAGA |
| | | | CTCTGGAAAGCCGACGTACACGGCACTGTACAAGCTACATTTAT |
| | | | CTTGAAGGACGCTTTCGCCGGCGGCGTTAAGCCCTTCGAACTGC |
| | | | ATCCCAGATTGGAGTCCCCCAATAGCGGTTCCTGTTCCCTTCCA |
| | | | GAGAGGCATCTGGGTCTTGTTTCCTGTTTCTTTCAGGAAGGCTG |
| | | | GGTGCTCAGCTGGAACGAATACTCAATATACCTGCTTGATACTG |
| | | | TGAACCAAGCTACTGTTGCGGGGCTGGAGGGAAGCGGTGATAT |
| | | | TGTCAGCGTGTCATGCACTGAAAATGAGATTTTTTTTCTCAAAG |
| | | | GGGATCGAAATATAATTCGGATCTCATCCCGGCCCGAAGGACTT |
| | | | ACTAGCACTGTAAGAGACGGACTGGAGATGAGCGGATGTTCCG |
| | | | AACGCGTGCACGTACAACAGGCTGAGAAGCTGCCCGGAGCTAC |
| | | | TGTGAGTGAAACACGGTTGCGGGGCTCATCCATGGCTTCTTCAG |
| | | | TTGCAAGCGAACCGCGATCACGGTCATCATCATTGAACTCAACT |
| | | | GATAGCGGCAGTGGACTGCTTCCCCCGGGATTGCAGGCTACCCC |
| | | | CGAACTCGGTAAAGGTTCTCAACCTCTCTCTCAGCGCTTCAACG |
| | | | CGATCTCCTCAGAAGATTTCGACCAGGAGTTGGTTGTTAAGCCC |
| | | | ATCAAAGTCAAGCGCAAAAAGAAAAAAAAAAAGACCGAAGGG |
| | | | GGCTCCCGGAGTACGTGCCACTCCTCTCTTGAGAGTACTCCGTG |
| | | | TAGTGAGTTTCCAGGCGACAGTCCGCAGTCTCTGAACACAGACC |
| | | | TTTTGAGTATGACAAGCTCAGTGCTTGGGAGCAGTGTAGACCAA |
| | | | CTTTCAGCTGAGTCACCGGATCAGGAGTCTTCTTTCAATGGCGA |
| | | | GGTTAATGGTGTACCACAGGAGAATACAGATCCCGAAACATTC |
| | | | AACGTCCTCGAAGTCTCTGGTAGTATGCCAGATTCCCTCGCTGA |
| | | | AGAAGATGACATACGGACGGAAATGCCCCATTGCCACCACGCG |
| | | | CATGGTCGAGAGCTTCTTAACGGAGCAAGGGAAGACGTAGGGG |
| | | | GCTCCGACGTAACGGGGTTGGGTGATGAACCGTGCCCGGCTGAC |
| | | | GACGGTCCCAATAGTACACAGTTGCCGTTCCAAGAACAGGACA |
| | | | GTTCCCCCGGCGCGCATGATGGAGAGGACATCCAGCCGATAGG |
| | | | TCCACAAAGTACTTTCTGCGAGGTTCCACTTCTCAACTCACTCAC |
| | | | TGTCCCGAGCTCTCTTTCATGGGCCCCGTCAGCAGAACAATGGT |
| | | | TGCCAGGAACGCGCGCTGATGAAGGCTCCCCGGTTGAACCATCC |
| | | | CAAGAGCAGGACATACTGACATCCATGGAAGCTAGTGGGCACC |
| | | | TTTCTACTAATCTGTGGCACGCGGTCACGGATGACGATACGGGT |
| | | | CAGAAGGAGATACCAATCTCCGAGCGCGTACTCGGCTCCGTAG |
| | | | GCGGACAACTTACTCCCGTATCTGCGCTCGCAGCCAGTACCCAC |
| | | | AAGCCATGGCTCGAGCAGCCACCTCGAGATCAAACTCTGACGTC |
| | | | ATCAGATGAAGAGGACATTTACGCCCACGGGCTGCCCTCTTCTT |
| | | | CCTCAGAGACATCCGTTACAGAGTTGGGTCCGTCTTGCTCACAA |
| | | | CAAGATTTGTCAAGACTTGGTGCCGAAGATGCTGGTCTCCTGAA |
| | | | ACCTGATCAGTTTGCAGAGTCCTGGATGGGCTACTCTGGCCCGG |
| | | | GCTATGGGATACTCTCCCTGGTCGTCTCCGAAAAGTATATTTGG |
| | | | TGTCTTGATTATAAGGGGGGTCTCTTCTGTTCTGCATTGCCGGGA |
| | | | GCAGGCTTGAGATGGCAAAAATTCGAGGATGCAGTACAACAAG |
| | | | TAGCCGTATCACCTAGTGGAGCATTGCTGTGGAAGATTGAGCAA |
| | | | AAATCAAATCGGGCGTTTGCTTGTGGAAAGGTAACGATTAAAG |
| | | | GTAAGCGACACTGGTACGAAGCACTTCCCCAGGCCGTATTTGTT |
| | | | GCGCTGTCCGATGACACCGCCTGGATCATTCGAACATCTGGGGA |
| | | | TTTGTATCTTCAAACGGGGCTCAGCGTAGATAGACCTTGTGCTC |
| | | | GGGCGGTAAAAGTGGACTGCCCATACCCCTTGTCACAGATTACT |
| | | | GCACGAAACAACGTCGTTTGGGCTCTGACGGAGCAACGAGCTCT |
| | | | GCTCTATAGGGAAGGTGTAAGCTCTTTCTGCCCAGAAGGGGAAC |
| | | | AGTGGAAGTGTGATATCGTATCAGAGCGCCAAGCTCTCGAACCC |
| | | | GTATGTATCACACTGGGTGATCAGCAAACGCTTTGGGCTCTTGA |
| | | | CATACATGGAAATCTTTGGTTTCGCACAGGCATCATTTCAAAAA |
| | | | AACCACAGGGAGACGACGACCACTGGTGGCAAGTTTCTATCAC |
| | | | GGATTATGTCGTATTCGATCAGTGTTCACTTTTCCAGACCATAAT |
| | | | CCATGCCACGCATTCCGTGGCAACCGCGGCTCAAGCTCCCGTAG |
| | | | AAAAGGTGGCAGATAAACTCCGCATGGCATTTTGGTCACAACA |
| | | | GCTTCAGTGCCAACCTTCTTTGCTTGGTGTGAATAACTCTGGAGT |
| | | | TTGGATTTCCAGTGGTAAAAACGAATTCCACGTTGCTAAGGGCA |
| | | | GCCTTATTGGTACTTATTGGAATCATGTTGTCCCTAGAGGAACA |
| | | | GCCTCTGCGACCAAGTGGGCCTTCGTACTTGCATCAGCGGCGCC |
| | | | AACTAAGGAGGGTTCATTCCTCTGGCTTTGCCAGTCTAGTAAAG |
| | | | ACCTCTGTAGTGTGTCCGCACAAAGCGCCCAAAGCCGACCAAGC |
| | | | ACCGTGCAGCTGCCGCCGGAGGCCGAGATGAGGGCGTACGCCG |
| | | | CATGTCAAGACGCTTTGTGGGCGCTTGATTCTCTGGGTCAGGTA |
| | | | TTTATTAGGACTCTTTCAAAGAGCTGCCCCACGGGCATGCACTG |
| | | | GACGCGGCTGGATCTCTCTCAGCTTGGGGCCGTAAAGCTCACTT |
| | | | CTCTTGCGTGTGGCAACCAGCATATCTGGGCATGCGATTCCAGA |
| | | | GGGGGAGTATACTTTCGAGTGGGCACACAACCTCTTAATCCGTC |
| | | | ACTGATGCTTCCAGCGTGGATAATGATCGAGCCGCCGGTCCAAC |
| | | | CCGCGGGTGTTTCACTTGTTAGTGTTCATTCATCTCCGAATGACC |
| | | | AAATGCTCTGGGTCCTCGACTCTAGATGGAATGTTCACGTACGG |
| | | | ACGGGTATTACAGAGGAGATGCCTGTAGGTACGGCTTGGGAGC |
| | | | ATGTCCCTGGCCTGCAGGCATGTCAACTGGCTCTGTCCACTAGG |
| | | | ACGGTCTGGGCCAGGTGTCCAAATGGGGACCTTGCCAGACGGT |
| | | | ACGGAGTGACAGACAAGAATCCTGCAGGTGACTATTGGAAGAA |
| | | | AATACCCGGTAGTGTAAGCTGTTTCACCGTCACGGCCTCAGATG |
| | | | AACTGTGGGCTGTGGGACCACCTGGTTACCTCCTCCAGCGCTTG |
| | | | ACTAAGACATTCTCCCATTCCCACGGCACCCAGAAAAGTTCACA |
| | | | AGCTGCTATGCCACATCCAGAAGACCTGGAAGATGAGTGGGAA |
| | | | GTTATTTGACCTTAACTTGTTTATTGCAGCTTATAATGGTTACAA |
| | | | ATAAAGCAATAGCATCACAAATTTCACAAATAAAGCATTTTTTT |
| | | | CACTGCATTCTAGTTGTGGTTTGTCCAAACTCATCAATGTATCTT |
| | | | A |
| 2 | Promoter: | 9 | GGGCGGAGTTAGGGCGGAGCCAATCAGCGTGCGCCGTTCCGAA |
| | JeT | | AGTTGCCTTTTATGGCTGGGCGGAGAATGGGCGGTGAACGCCGA |
| | Chimeric | | TGATTATATAAGGACGCGCCGGGTGTGGCACAGCTAGTTCCGTC |
| | Intron | | GCAGCCGGGATTTGGGTCGCGGTTCTTGTTTGTGCGGCCGCCGC |
| | Coding | | ATGGCGAGCATCTCAGAGCCGGTCACGTTTAGAGAGTTCTGTCC |
| | sequence: | | ACTTTATTACCTTCTTAATGCAATTCCAACTAAAATCCAGAAAG |
| | TECPR2 Iso1 | | GATTTCGATCTATAGTAGTTTATTTGACCGCATTGGATACGAAC |
| | (codon | | GGCGACTACATCGCGGTTGGCTCAAGCATCGGAATGCTCTATTT |
| | optimized) | | GTACTGTCGGCACCTCAATCAGATGAGGAAATATAACTTCGAAG |
| | PolyA Signal: | | GCAAGACCGAATCCATTACAGTTGTGAAGCTCCTCTCCTGTTTC |
| | SV40 | | GACGACCTGGTGGCCGCCGGCACCGCCTCCGGCCGAGTTGCAGT |
| | | | GTTCCAACTTGTATCAAGCCTCCCCGGCAGGAACAAGCAACTGA |
| | | | GAAGATTTGACGTTACGGGTATTCATAAGAATTCCATAACGGCT |
| | | | CTGGCATGGAGCCCGAACGGGATGAAACTGTTCAGCGGTGACG |
| | | | ATAAAGGGAAGATCGTGTACTCTTCTCTGGATCTGGACCAAGGG |
| | | | CTTTGCAACTCCCAACTTGTTCTCGAGGAACCATCTTCTATCGTC |
| | | | CAGCTTGACTACAGTCAGAAGGTCTTGCTTGTGTCAACCCTTCA |
| | | | GCGAAGCCTTCTTTTTTATACGGAAGAGAAGTCCGTTCGGCAGA |
| | | | TTGGGACGCAACCCCGCAAAAGCACCGGTAAGTTTGGCGCATGT |
| | | | TTTATCCCAGGACTGTGTAAACAATCTGACTTGACACTTTATGCC |
| | | | TCACGCCCAGGTCTGAGACTCTGGAAAGCCGACGTACACGGCA |
| | | | CTGTACAAGCTACATTTATCTTGAAGGACGCTTTCGCCGGCGGC |
| | | | GTTAAGCCCTTCGAACTGCATCCCAGATTGGAGTCCCCCAATAG |
| | | | CGGTTCCTGTTCCCTTCCAGAGAGGCATCTGGGTCTTGTTTCCTG |
| | | | TTTCTTTCAGGAAGGCTGGGTGCTCAGCTGGAACGAATACTCAA |
| | | | TATACCTGCTTGATACTGTGAACCAAGCTACTGTTGCGGGGCTG |
| | | | GAGGGAAGCGGTGATATTGTCAGCGTGTCATGCACTGAAAATG |
| | | | AGATTTTTTTTCTCAAAGGGGATCGAAATATAATTCGGATCTCA |
| | | | TCCCGGCCCGAAGGACTTACTAGCACTGTAAGAGACGGACTGG |
| | | | AGATGAGCGGATGTTCCGAACGCGTGCACGTACAACAGGCTGA |
| | | | GAAGCTGCCCGGAGCTACTGTGAGTGAAACACGGTTGCGGGGC |
| | | | TCATCCATGGCTTCTTCAGTTGCAAGCGAACCGCGATCACGGTC |
| | | | ATCATCATTGAACTCAACTGATAGCGGCAGTGGACTGCTTCCCC |
| | | | CGGGATTGCAGGCTACCCCCGAACTCGGTAAAGGTTCTCAACCT |
| | | | CTCTCTCAGCGCTTCAACGCGATCTCCTCAGAAGATTTCGACCA |
| | | | GGAGTTGGTTGTTAAGCCCATCAAAGTCAAGCGCAAAAAGAAA |
| | | | AAAAAAAAGACCGAAGGGGGCTCCCGGAGTACGTGCCACTCCT |
| | | | CTCTTGAGAGTACTCCGTGTAGTGAGTTTCCAGGCGACAGTCCG |
| | | | CAGTCTCTGAACACAGACCTTTTGAGTATGACAAGCTCAGTGCT |
| | | | TGGGAGCAGTGTAGACCAACTTTCAGCTGAGTCACCGGATCAGG |
| | | | AGTCTTCTTTCAATGGCGAGGTTAATGGTGTACCACAGGAGAAT |
| | | | ACAGATCCCGAAACATTCAACGTCCTCGAAGTCTCTGGTAGTAT |
| | | | GCCAGATTCCCTCGCTGAAGAAGATGACATACGGACGGAAATG |
| | | | CCCCATTGCCACCACGCGCATGGTCGAGAGCTTCTTAACGGAGC |
| | | | AAGGGAAGACGTAGGGGGCTCCGACGTAACGGGGTTGGGTGAT |
| | | | GAACCGTGCCCGGCTGACGACGGTCCCAATAGTACACAGTTGCC |
| | | | GTTCCAAGAACAGGACAGTTCCCCCGGCGCGCATGATGGAGAG |
| | | | GACATCCAGCCGATAGGTCCACAAAGTACTTTCTGCGAGGTTCC |
| | | | ACTTCTCAACTCACTCACTGTCCCGAGCTCTCTTTCATGGGCCCC |
| | | | GTCAGCAGAACAATGGTTGCCAGGAACGCGCGCTGATGAAGGC |
| | | | TCCCCGGTTGAACCATCCCAAGAGCAGGACATACTGACATCCAT |
| | | | GGAAGCTAGTGGGCACCTTTCTACTAATCTGTGGCACGCGGTCA |
| | | | CGGATGACGATACGGGTCAGAAGGAGATACCAATCTCCGAGCG |
| | | | CGTACTCGGCTCCGTAGGCGGACAACTTACTCCCGTATCTGCGC |
| | | | TCGCAGCCAGTACCCACAAGCCATGGCTCGAGCAGCCACCTCGA |
| | | | GATCAAACTCTGACGTCATCAGATGAAGAGGACATTTACGCCCA |
| | | | CGGGCTGCCCTCTTCTTCCTCAGAGACATCCGTTACAGAGTTGG |
| | | | GTCCGTCTTGCTCACAACAAGATTTGTCAAGACTTGGTGCCGAA |
| | | | GATGCTGGTCTCCTGAAACCTGATCAGTTTGCAGAGTCCTGGAT |
| | | | GGGCTACTCTGGCCCGGGCTATGGGATACTCTCCCTGGTCGTCT |
| | | | CCGAAAAGTATATTTGGTGTCTTGATTATAAGGGGGGTCTCTTC |
| | | | TGTTCTGCATTGCCGGGAGCAGGCTTGAGATGGCAAAAATTCGA |
| | | | GGATGCAGTACAACAAGTAGCCGTATCACCTAGTGGAGCATTGC |
| | | | TGTGGAAGATTGAGCAAAAATCAAATCGGGCGTTTGCTTGTGGA |
| | | | AAGGTAACGATTAAAGGTAAGCGACACTGGTACGAAGCACTTC |
| | | | CCCAGGCCGTATTTGTTGCGCTGTCCGATGACACCGCCTGGATC |
| | | | ATTCGAACATCTGGGGATTTGTATCTTCAAACGGGGCTCAGCGT |
| | | | AGATAGACCTTGTGCTCGGGCGGTAAAAGTGGACTGCCCATACC |
| | | | CCTTGTCACAGATTACTGCACGAAACAACGTCGTTTGGGCTCTG |
| | | | ACGGAGCAACGAGCTCTGCTCTATAGGGAAGGTGTAAGCTCTTT |
| | | | CTGCCCAGAAGGGGAACAGTGGAAGTGTGATATCGTATCAGAG |
| | | | CGCCAAGCTCTCGAACCCGTATGTATCACACTGGGTGATCAGCA |
| | | | AACGCTTTGGGCTCTTGACATACATGGAAATCTTTGGTTTCGCA |
| | | | CAGGCATCATTTCAAAAAAACCACAGGGAGACGACGACCACTG |
| | | | GTGGCAAGTTTCTATCACGGATTATGTCGTATTCGATCAGTGTTC |
| | | | ACTTTTCCAGACCATAATCCATGCCACGCATTCCGTGGCAACCG |
| | | | CGGCTCAAGCTCCCGTAGAAAAGGTGGCAGATAAACTCCGCAT |
| | | | GGCATTTTGGTCACAACAGCTTCAGTGCCAACCTTCTTTGCTTGG |
| | | | TGTGAATAACTCTGGAGTTTGGATTTCCAGTGGTAAAAACGAAT |
| | | | TCCACGTTGCTAAGGGCAGCCTTATTGGTACTTATTGGAATCAT |
| | | | GTTGTCCCTAGAGGAACAGCCTCTGCGACCAAGTGGGCCTTCGT |
| | | | ACTTGCATCAGCGGCGCCAACTAAGGAGGGTTCATTCCTCTGGC |
| | | | TTTGCCAGTCTAGTAAAGACCTCTGTAGTGTGTCCGCACAAAGC |
| | | | GCCCAAAGCCGACCAAGCACCGTGCAGCTGCCGCCGGAGGCCG |
| | | | AGATGAGGGCGTACGCCGCATGTCAAGACGCTTTGTGGGCGCTT |
| | | | GATTCTCTGGGTCAGGTATTTATTAGGACTCTTTCAAAGAGCTG |
| | | | CCCCACGGGCATGCACTGGACGCGGCTGGATCTCTCTCAGCTTG |
| | | | GGGCCGTAAAGCTCACTTCTCTTGCGTGTGGCAACCAGCATATC |
| | | | TGGGCATGCGATTCCAGAGGGGGAGTATACTTTCGAGTGGGCAC |
| | | | ACAACCTCTTAATCCGTCACTGATGCTTCCAGCGTGGATAATGA |
| | | | TCGAGCCGCCGGTCCAACCCGCGGGTGTTTCACTTGTTAGTGTT |
| | | | CATTCATCTCCGAATGACCAAATGCTCTGGGTCCTCGACTCTAG |
| | | | ATGGAATGTTCACGTACGGACGGGTATTACAGAGGAGATGCCT |
| | | | GTAGGTACGGCTTGGGAGCATGTCCCTGGCCTGCAGGCATGTCA |
| | | | ACTGGCTCTGTCCACTAGGACGGTCTGGGCCAGGTGTCCAAATG |
| | | | GGGACCTTGCCAGACGGTACGGAGTGACAGACAAGAATCCTGC |
| | | | AGGTGACTATTGGAAGAAAATACCCGGTAGTGTAAGCTGTTTCA |
| | | | CCGTCACGGCCTCAGATGAACTGTGGGCTGTGGGACCACCTGGT |
| | | | TACCTCCTCCAGCGCTTGACTAAGACATTCTCCCATTCCCACGGC |
| | | | ACCCAGAAAAGTTCACAAGCTGCTATGCCACATCCAGAAGACCT |
| | | | GGAAGATGAGTGGGAAGTTATTTGACCTTAACTTGTTTATTGCA |
| | | | GCTTATAATGGTTACAAATAAAGCAATAGCATCACAAATTTCAC |
| | | | AAATAAAGCATTTTTTTCACTGCATTCTAGTTGTGGTTTGTCCAA |
| | | | ACTCATCAATGTATCTTA |
| 3 | Promoter: | 11 | GCTGCAGAGGGCCCTGCGTATGAGTGCAAGTGGGTTTTAGGACC |
| | Human | | AGGATGAGGCGGGGTGGGGGTGCCTACCTGACGACCGACCCCG |
| | Synapsin | | ACCCACTGGACAAGCACCCAACCCCCATTCCCCAAATTGCGCAT |
| | (hSyn) | | CCCCTATCAGAGAGGGGGAGGGGAAACAGGATGCGGCGAGGCG |
| | MVM Intron | | CGTGCGCACTGCCAGCTTCAGCACCGCGGACAGTGCCTTCGCCC |
| | Coding | | CCGCCTGGCGGCGCGCGCCACCGCCGCCTCAGCACTGAAGGCG |
| | sequence: | | CGCTGACGTCACTCGCCGGTCCCCCGCAAACTCCCCTTCCCGGC |
| | TECPR2 Iso1 | | CACCTTGGTCGCGTCCGCGCCGCCGCCGGCCCAGCCGGACCGCA |
| | (codon | | CCACGCGAGGCGCGAGATAGGGGGGCACGGGCGCGACCATCTG |
| | optimized) | | CGCTGCGGCGCCGGCGTCGACAAGAGGTAAGGGTTTAAGGGAT |
| | PolyA Signal: | | GGTTGGTTGGTGGGGTATTAATGTTTAATTACCTGTTTTACAGGC |
| | SV40 | | CTGAAATCACTTGGTTTTAGGTTGGGAAGCTTGATATCCCTGCA |
| | | | GGGCGGCCGCCGCATGGCGAGCATCTCAGAGCCGGTCACGTTTA |
| | | | GAGAGTTCTGTCCACTTTATTACCTTCTTAATGCAATTCCAACTA |
| | | | AAATCCAGAAAGGATTTCGATCTATAGTAGTTTATTTGACCGCA |
| | | | TTGGATACGAACGGCGACTACATCGCGGTTGGCTCAAGCATCGG |
| | | | AATGCTCTATTTGTACTGTCGGCACCTCAATCAGATGAGGAAAT |
| | | | ATAACTTCGAAGGCAAGACCGAATCCATTACAGTIGTGAAGCTC |
| | | | CTCTCCTGTTTCGACGACCTGGTGGCCGCCGGCACCGCCTCCGG |
| | | | CCGAGTTGCAGTGTTCCAACTTGTATCAAGCCTCCCCGGCAGGA |
| | | | ACAAGCAACTGAGAAGATTTGACGTTACGGGTATTCATAAGAAT |
| | | | TCCATAACGGCTCTGGCATGGAGCCCGAACGGGATGAAACTGTT |
| | | | CAGCGGTGACGATAAAGGGAAGATCGTGTACTCTTCTCTGGATC |
| | | | TGGACCAAGGGCTTTGCAACTCCCAACTTGTTCTCGAGGAACCA |
| | | | TCTTCTATCGTCCAGCTTGACTACAGTCAGAAGGTCTTGCTTGTG |
| | | | TCAACCCTTCAGCGAAGCCTTCTTTTTTATACGGAAGAGAAGTC |
| | | | CGTTCGGCAGATTGGGACGCAACCCCGCAAAAGCACCGGTAAG |
| | | | TTTGGCGCATGTTTTATCCCAGGACTGTGTAAACAATCTGACTTG |
| | | | ACACTTTATGCCTCACGCCCAGGTCTGAGACTCTGGAAAGCCGA |
| | | | CGTACACGGCACTGTACAAGCTACATTTATCTTGAAGGACGCTT |
| | | | TCGCCGGCGGCGTTAAGCCCTTCGAACTGCATCCCAGATTGGAG |
| | | | TCCCCCAATAGCGGTTCCTGTTCCCTTCCAGAGAGGCATCTGGG |
| | | | TCTTGTTTCCTGTTTCTTTCAGGAAGGCTGGGTGCTCAGCTGGAA |
| | | | CGAATACTCAATATACCTGCTTGATACTGTGAACCAAGCTACTG |
| | | | TTGCGGGGCTGGAGGGAAGCGGTGATATTGTCAGCGTGTCATGC |
| | | | ACTGAAAATGAGATTTTTTTTCTCAAAGGGGATCGAAATATAAT |
| | | | TCGGATCTCATCCCGGCCCGAAGGACTTACTAGCACTGTAAGAG |
| | | | ACGGACTGGAGATGAGCGGATGTTCCGAACGCGTGCACGTACA |
| | | | ACAGGCTGAGAAGCTGCCCGGAGCTACTGTGAGTGAAACACGG |
| | | | TTGCGGGGCTCATCCATGGCTTCTTCAGTTGCAAGCGAACCGCG |
| | | | ATCACGGTCATCATCATTGAACTCAACTGATAGCGGCAGTGGAC |
| | | | TGCTTCCCCCGGGATTGCAGGCTACCCCCGAACTCGGTAAAGGT |
| | | | TCTCAACCTCTCTCTCAGCGCTTCAACGCGATCTCCTCAGAAGAT |
| | | | TTCGACCAGGAGTTGGTTGTTAAGCCCATCAAAGTCAAGCGCAA |
| | | | AAAGAAAAAAAAAAAGACCGAAGGGGGCTCCCGGAGTACGTG |
| | | | CCACTCCTCTCTTGAGAGTACTCCGTGTAGTGAGTTTCCAGGCG |
| | | | ACAGTCCGCAGTCTCTGAACACAGACCTTTTGAGTATGACAAGC |
| | | | TCAGTGCTTGGGAGCAGTGTAGACCAACTTTCAGCTGAGTCACC |
| | | | GGATCAGGAGTCTTCTTTCAATGGCGAGGTTAATGGTGTACCAC |
| | | | AGGAGAATACAGATCCCGAAACATTCAACGTCCTCGAAGTCTCT |
| | | | GGTAGTATGCCAGATTCCCTCGCTGAAGAAGATGACATACGGAC |
| | | | GGAAATGCCCCATTGCCACCACGCGCATGGTCGAGAGCTTCTTA |
| | | | ACGGAGCAAGGGAAGACGTAGGGGGCTCCGACGTAACGGGGTT |
| | | | GGGTGATGAACCGTGCCCGGCTGACGACGGTCCCAATAGTACA |
| | | | CAGTTGCCGTTCCAAGAACAGGACAGTTCCCCCGGCGCGCATGA |
| | | | TGGAGAGGACATCCAGCCGATAGGTCCACAAAGTACTTTCTGCG |
| | | | AGGTTCCACTTCTCAACTCACTCACTGTCCCGAGCTCTCTTTCAT |
| | | | GGGCCCCGTCAGCAGAACAATGGTTGCCAGGAACGCGCGCTGA |
| | | | TGAAGGCTCCCCGGTTGAACCATCCCAAGAGCAGGACATACTG |
| | | | ACATCCATGGAAGCTAGTGGGCACCTTTCTACTAATCTGTGGCA |
| | | | CGCGGTCACGGATGACGATACGGGTCAGAAGGAGATACCAATC |
| | | | TCCGAGCGCGTACTCGGCTCCGTAGGCGGACAACTTACTCCCGT |
| | | | ATCTGCGCTCGCAGCCAGTACCCACAAGCCATGGCTCGAGCAGC |
| | | | CACCTCGAGATCAAACTCTGACGTCATCAGATGAAGAGGACATT |
| | | | TACGCCCACGGGCTGCCCTCTTCTTCCTCAGAGACATCCGTTAC |
| | | | AGAGTTGGGTCCGTCTTGCTCACAACAAGATTTGTCAAGACTTG |
| | | | GTGCCGAAGATGCTGGTCTCCTGAAACCTGATCAGTTTGCAGAG |
| | | | TCCTGGATGGGCTACTCTGGCCCGGGCTATGGGATACTCTCCCT |
| | | | GGTCGTCTCCGAAAAGTATATTTGGTGTCTTGATTATAAGGGGG |
| | | | GTCTCTTCTGTTCTGCATTGCCGGGAGCAGGCTTGAGATGGCAA |
| | | | AAATTCGAGGATGCAGTACAACAAGTAGCCGTATCACCTAGTG |
| | | | GAGCATTGCTGTGGAAGATTGAGCAAAAATCAAATCGGGCGTTT |
| | | | GCTTGTGGAAAGGTAACGATTAAAGGTAAGCGACACTGGTACG |
| | | | AAGCACTTCCCCAGGCCGTATTTGTTGCGCTGTCCGATGACACC |
| | | | GCCTGGATCATTCGAACATCTGGGGATTTGTATCTTCAAACGGG |
| | | | GCTCAGCGTAGATAGACCTTGTGCTCGGGCGGTAAAAGTGGACT |
| | | | GCCCATACCCCTTGTCACAGATTACTGCACGAAACAACGTCGTT |
| | | | TGGGCTCTGACGGAGCAACGAGCTCTGCTCTATAGGGAAGGTGT |
| | | | AAGCTCTTTCTGCCCAGAAGGGGAACAGTGGAAGTGTGATATCG |
| | | | TATCAGAGCGCCAAGCTCTCGAACCCGTATGTATCACACTGGGT |
| | | | GATCAGCAAACGCTTTGGGCTCTTGACATACATGGAAATCTTTG |
| | | | GTTTCGCACAGGCATCATTTCAAAAAAACCACAGGGAGACGAC |
| | | | GACCACTGGTGGCAAGTTTCTATCACGGATTATGTCGTATTCGA |
| | | | TCAGTGTTCACTTTTCCAGACCATAATCCATGCCACGCATTCCGT |
| | | | GGCAACCGCGGCTCAAGCTCCCGTAGAAAAGGTGGCAGATAAA |
| | | | CTCCGCATGGCATTTTGGTCACAACAGCTTCAGTGCCAACCTTCT |
| | | | TTGCTTGGTGTGAATAACTCTGGAGTTTGGATTTCCAGTGGTAA |
| | | | AAACGAATTCCACGTTGCTAAGGGCAGCCTTATTGGTACTTATT |
| | | | GGAATCATGTTGTCCCTAGAGGAACAGCCTCTGCGACCAAGTGG |
| | | | GCCTTCGTACTTGCATCAGCGGCGCCAACTAAGGAGGGTTCATT |
| | | | CCTCTGGCTTTGCCAGTCTAGTAAAGACCTCTGTAGTGTGTCCGC |
| | | | ACAAAGCGCCCAAAGCCGACCAAGCACCGTGCAGCTGCCGCCG |
| | | | GAGGCCGAGATGAGGGCGTACGCCGCATGTCAAGACGCTTTGT |
| | | | GGGCGCTTGATTCTCTGGGTCAGGTATTTATTAGGACTCTTTCAA |
| | | | AGAGCTGCCCCACGGGCATGCACTGGACGCGGCTGGATCTCTCT |
| | | | CAGCTTGGGGCCGTAAAGCTCACTTCTCTTGCGTGTGGCAACCA |
| | | | GCATATCTGGGCATGCGATTCCAGAGGGGGAGTATACTTTCGAG |
| | | | TGGGCACACAACCTCTTAATCCGTCACTGATGCTTCCAGCGTGG |
| | | | ATAATGATCGAGCCGCCGGTCCAACCCGCGGGTGTTTCACTTGT |
| | | | TAGTGTTCATTCATCTCCGAATGACCAAATGCTCTGGGTCCTCG |
| | | | ACTCTAGATGGAATGTTCACGTACGGACGGGTATTACAGAGGA |
| | | | GATGCCTGTAGGTACGGCTTGGGAGCATGTCCCTGGCCTGCAGG |
| | | | CATGTCAACTGGCTCTGTCCACTAGGACGGTCTGGGCCAGGTGT |
| | | | CCAAATGGGGACCTTGCCAGACGGTACGGAGTGACAGACAAGA |
| | | | ATCCTGCAGGTGACTATTGGAAGAAAATACCCGGTAGTGTAAGC |
| | | | TGTTTCACCGTCACGGCCTCAGATGAACTGTGGGCTGTGGGACC |
| | | | ACCTGGTTACCTCCTCCAGCGCTTGACTAAGACATTCTCCCATTC |
| | | | CCACGGCACCCAGAAAAGTTCACAAGCTGCTATGCCACATCCAG |
| | | | AAGACCTGGAAGATGAGTGGGAAGTTATTTGACCTTAACTTGTT |
| | | | TATTGCAGCTTATAATGGTTACAAATAAAGCAATAGCATCACAA |
| | | | ATTTCACAAATAAAGCATTTTTTTCACTGCATTCTAGTTGTGGTT |
| | | | TGTCCAAACTCATCAATGTATCTTA |
| 4 | Promoter: | 13 | CAAAATCAACGGGACTTTCCAAAATGTCGTAATAACCCCGCCCC |
| | Minimum | | GTTGACGCAAATGGGCGGTAGGCGTGTACGGTGGGAGGTCTAT |
| | CMV | | ATAAGCAGAGCTCGTTTAGTGAACCGTCAGAATTCTCGAGTGAT |
| | (minCMV) | | CGAAAGAGCCTGCTAAAGCAAAAAAGAAGTCACCGTCGACAAG |
| | MVM Intron | | AGGTAAGGGTTTAAGGGATGGTTGGTTGGTGGGGTATTAATGTT |
| | (MVMi) | | TAATTACCTGTTTTACAGGCCTGAAATCACTTGGTTTTAGGTTGG |
| | Coding | | GAAGCTTGATATCCCTGCAGGGCGGCCGCCGCATGGCGAGCATC |
| | sequence: | | TCAGAGCCGGTCACGTTTAGAGAGTTCTGTCCACTTTATTACCTT |
| | TECPR2 Iso1 | | CTTAATGCAATTCCAACTAAAATCCAGAAAGGATTTCGATCTAT |
| | (codon | | AGTAGTTTATTTGACCGCATTGGATACGAACGGCGACTACATCG |
| | optimized) | | CGGTTGGCTCAAGCATCGGAATGCTCTATTTGTACTGTCGGCAC |
| | PolyA Signal: | | CTCAATCAGATGAGGAAATATAACTTCGAAGGCAAGACCGAAT |
| | SV40 | | CCATTACAGTIGTGAAGCTCCTCTCCTGTTTCGACGACCTGGTGG |
| | | | CCGCCGGCACCGCCTCCGGCCGAGTTGCAGTGTTCCAACTTGTA |
| | | | TCAAGCCTCCCCGGCAGGAACAAGCAACTGAGAAGATTTGACG |
| | | | TTACGGGTATTCATAAGAATTCCATAACGGCTCTGGCATGGAGC |
| | | | CCGAACGGGATGAAACTGTTCAGCGGTGACGATAAAGGGAAGA |
| | | | TCGTGTACTCTTCTCTGGATCTGGACCAAGGGCTTTGCAACTCCC |
| | | | AACTTGTTCTCGAGGAACCATCTTCTATCGTCCAGCTTGACTACA |
| | | | GTCAGAAGGTCTTGCTTGTGTCAACCCTTCAGCGAAGCCTTCTTT |
| | | | TTTATACGGAAGAGAAGTCCGTTCGGCAGATTGGGACGCAACCC |
| | | | CGCAAAAGCACCGGTAAGTTTGGCGCATGTTTTATCCCAGGACT |
| | | | GTGTAAACAATCTGACTTGACACTTTATGCCTCACGCCCAGGTC |
| | | | TGAGACTCTGGAAAGCCGACGTACACGGCACTGTACAAGCTAC |
| | | | ATTTATCTTGAAGGACGCTTTCGCCGGCGGCGTTAAGCCCTTCG |
| | | | AACTGCATCCCAGATTGGAGTCCCCCAATAGCGGTTCCTGTTCC |
| | | | CTTCCAGAGAGGCATCTGGGTCTTGTTTCCTGTTTCTTTCAGGAA |
| | | | GGCTGGGTGCTCAGCTGGAACGAATACTCAATATACCTGCTTGA |
| | | | TACTGTGAACCAAGCTACTGTTGCGGGGCTGGAGGGAAGCGGT |
| | | | GATATTGTCAGCGTGTCATGCACTGAAAATGAGATTTTTTTTCTC |
| | | | AAAGGGGATCGAAATATAATTCGGATCTCATCCCGGCCCGAAG |
| | | | GACTTACTAGCACTGTAAGAGACGGACTGGAGATGAGCGGATG |
| | | | TTCCGAACGCGTGCACGTACAACAGGCTGAGAAGCTGCCCGGA |
| | | | GCTACTGTGAGTGAAACACGGTTGCGGGGCTCATCCATGGCTTC |
| | | | TTCAGTTGCAAGCGAACCGCGATCACGGTCATCATCATTGAACT |
| | | | CAACTGATAGCGGCAGTGGACTGCTTCCCCCGGGATTGCAGGCT |
| | | | ACCCCCGAACTCGGTAAAGGTTCTCAACCTCTCTCTCAGCGCTT |
| | | | CAACGCGATCTCCTCAGAAGATTTCGACCAGGAGTTGGTTGTTA |
| | | | AGCCCATCAAAGTCAAGCGCAAAAAGAAAAAAAAAAAGACCG |
| | | | AAGGGGGCTCCCGGAGTACGTGCCACTCCTCTCTTGAGAGTACT |
| | | | CCGTGTAGTGAGTTTCCAGGCGACAGTCCGCAGTCTCTGAACAC |
| | | | AGACCTTTTGAGTATGACAAGCTCAGTGCTTGGGAGCAGTGTAG |
| | | | ACCAACTTTCAGCTGAGTCACCGGATCAGGAGTCTTCTTTCAAT |
| | | | GGCGAGGTTAATGGTGTACCACAGGAGAATACAGATCCCGAAA |
| | | | CATTCAACGTCCTCGAAGTCTCTGGTAGTATGCCAGATTCCCTC |
| | | | GCTGAAGAAGATGACATACGGACGGAAATGCCCCATTGCCACC |
| | | | ACGCGCATGGTCGAGAGCTTCTTAACGGAGCAAGGGAAGACGT |
| | | | AGGGGGCTCCGACGTAACGGGGTTGGGTGATGAACCGTGCCCG |
| | | | GCTGACGACGGTCCCAATAGTACACAGTTGCCGTTCCAAGAACA |
| | | | GGACAGTTCCCCCGGCGCGCATGATGGAGAGGACATCCAGCCG |
| | | | ATAGGTCCACAAAGTACTTTCTGCGAGGTTCCACTTCTCAACTC |
| | | | ACTCACTGTCCCGAGCTCTCTTTCATGGGCCCCGTCAGCAGAAC |
| | | | AATGGTTGCCAGGAACGCGCGCTGATGAAGGCTCCCCGGTTGA |
| | | | ACCATCCCAAGAGCAGGACATACTGACATCCATGGAAGCTAGT |
| | | | GGGCACCTTTCTACTAATCTGTGGCACGCGGTCACGGATGACGA |
| | | | TACGGGTCAGAAGGAGATACCAATCTCCGAGCGCGTACTCGGCT |
| | | | CCGTAGGCGGACAACTTACTCCCGTATCTGCGCTCGCAGCCAGT |
| | | | ACCCACAAGCCATGGCTCGAGCAGCCACCTCGAGATCAAACTCT |
| | | | GACGTCATCAGATGAAGAGGACATTTACGCCCACGGGCTGCCCT |
| | | | CTTCTTCCTCAGAGACATCCGTTACAGAGTTGGGTCCGTCTTGCT |
| | | | CACAACAAGATTTGTCAAGACTTGGTGCCGAAGATGCTGGTCTC |
| | | | CTGAAACCTGATCAGTTTGCAGAGTCCTGGATGGGCTACTCTGG |
| | | | CCCGGGCTATGGGATACTCTCCCTGGTCGTCTCCGAAAAGTATA |
| | | | TTTGGTGTCTTGATTATAAGGGGGGTCTCTTCTGTTCTGCATTGC |
| | | | CGGGAGCAGGCTTGAGATGGCAAAAATTCGAGGATGCAGTACA |
| | | | ACAAGTAGCCGTATCACCTAGTGGAGCATTGCTGTGGAAGATTG |
| | | | AGCAAAAATCAAATCGGGCGTTTGCTTGTGGAAAGGTAACGATT |
| | | | AAAGGTAAGCGACACTGGTACGAAGCACTTCCCCAGGCCGTATT |
| | | | TGTTGCGCTGTCCGATGACACCGCCTGGATCATTCGAACATCTG |
| | | | GGGATTTGTATCTTCAAACGGGGCTCAGCGTAGATAGACCTTGT |
| | | | GCTCGGGCGGTAAAAGTGGACTGCCCATACCCCTTGTCACAGAT |
| | | | TACTGCACGAAACAACGTCGTTTGGGCTCTGACGGAGCAACGA |
| | | | GCTCTGCTCTATAGGGAAGGTGTAAGCTCTTTCTGCCCAGAAGG |
| | | | GGAACAGTGGAAGTGTGATATCGTATCAGAGCGCCAAGCTCTC |
| | | | GAACCCGTATGTATCACACTGGGTGATCAGCAAACGCTTTGGGC |
| | | | TCTTGACATACATGGAAATCTTTGGTTTCGCACAGGCATCATTTC |
| | | | AAAAAAACCACAGGGAGACGACGACCACTGGTGGCAAGTTTCT |
| | | | ATCACGGATTATGTCGTATTCGATCAGTGTTCACTTTTCCAGACC |
| | | | ATAATCCATGCCACGCATTCCGTGGCAACCGCGGCTCAAGCTCC |
| | | | CGTAGAAAAGGTGGCAGATAAACTCCGCATGGCATTTTGGTCAC |
| | | | AACAGCTTCAGTGCCAACCTTCTTTGCTTGGTGTGAATAACTCTG |
| | | | GAGTTTGGATTTCCAGTGGTAAAAACGAATTCCACGTTGCTAAG |
| | | | GGCAGCCTTATTGGTACTTATTGGAATCATGTTGTCCCTAGAGG |
| | | | AACAGCCTCTGCGACCAAGTGGGCCTTCGTACTTGCATCAGCGG |
| | | | CGCCAACTAAGGAGGGTTCATTCCTCTGGCTTTGCCAGTCTAGT |
| | | | AAAGACCTCTGTAGTGTGTCCGCACAAAGCGCCCAAAGCCGAC |
| | | | CAAGCACCGTGCAGCTGCCGCCGGAGGCCGAGATGAGGGCGTA |
| | | | CGCCGCATGTCAAGACGCTTTGTGGGCGCTTGATTCTCTGGGTC |
| | | | AGGTATTTATTAGGACTCTTTCAAAGAGCTGCCCCACGGGCATG |
| | | | CACTGGACGCGGCTGGATCTCTCTCAGCTTGGGGCCGTAAAGCT |
| | | | CACTTCTCTTGCGTGTGGCAACCAGCATATCTGGGCATGCGATT |
| | | | CCAGAGGGGGAGTATACTTTCGAGTGGGCACACAACCTCTTAAT |
| | | | CCGTCACTGATGCTTCCAGCGTGGATAATGATCGAGCCGCCGGT |
| | | | CCAACCCGCGGGTGTTTCACTTGTTAGTGTTCATTCATCTCCGAA |
| | | | TGACCAAATGCTCTGGGTCCTCGACTCTAGATGGAATGTTCACG |
| | | | TACGGACGGGTATTACAGAGGAGATGCCTGTAGGTACGGCTTG |
| | | | GGAGCATGTCCCTGGCCTGCAGGCATGTCAACTGGCTCTGTCCA |
| | | | CTAGGACGGTCTGGGCCAGGTGTCCAAATGGGGACCTTGCCAG |
| | | | ACGGTACGGAGTGACAGACAAGAATCCTGCAGGTGACTATTGG |
| | | | AAGAAAATACCCGGTAGTGTAAGCTGTTTCACCGTCACGGCCTC |
| | | | AGATGAACTGTGGGCTGTGGGACCACCTGGTTACCTCCTCCAGC |
| | | | GCTTGACTAAGACATTCTCCCATTCCCACGGCACCCAGAAAAGT |
| | | | TCACAAGCTGCTATGCCACATCCAGAAGACCTGGAAGATGAGT |
| | | | GGGAAGTTATTTGACCTTAACTTGTTTATTGCAGCTTATAATGGT |
| | | | TACAAATAAAGCAATAGCATCACAAATTTCACAAATAAAGCATT |
| | | | TTTTTCACTGCATTCTAGTTGTGGTTTGTCCAAACTCATCAATGT |
| | | | ATCTTA |
| 5 | Promoter: | 15 | TTCGCATATTAAGGTGACGCGTGTGGCCTCGAACACCGAGCGAC |
| | minTK | | CCTGCAGCGACCCGCTTAAAAGAGTCGACAAGAGGTAAGGGTT |
| | MVM Intron | | TAAGGGATGGTTGGTTGGTGGGGTATTAATGTTTAATTACCTGT |
| | Coding | | TTTACAGGCCTGAAATCACTTGGTTTTAGGTTGGGAAGCTTGAT |
| | sequence: | | ATCCCTGCAGGGCGGCCGCCGCATGGCGAGCATCTCAGAGCCG |
| | TECPR2 Iso1 | | GTCACGTTTAGAGAGTTCTGTCCACTTTATTACCTTCTTAATGCA |
| | (codon | | ATTCCAACTAAAATCCAGAAAGGATTTCGATCTATAGTAGTTTA |
| | optimized) | | TTTGACCGCATTGGATACGAACGGCGACTACATCGCGGTTGGCT |
| | PolyA Signal: | | CAAGCATCGGAATGCTCTATTTGTACTGTCGGCACCTCAATCAG |
| | SV40 | | ATGAGGAAATATAACTTCGAAGGCAAGACCGAATCCATTACAG |
| | | | TTGTGAAGCTCCTCTCCTGTTTCGACGACCTGGTGGCCGCCGGC |
| | | | ACCGCCTCCGGCCGAGTTGCAGTGTTCCAACTTGTATCAAGCCT |
| | | | CCCCGGCAGGAACAAGCAACTGAGAAGATTTGACGTTACGGGT |
| | | | ATTCATAAGAATTCCATAACGGCTCTGGCATGGAGCCCGAACGG |
| | | | GATGAAACTGTTCAGCGGTGACGATAAAGGGAAGATCGTGTAC |
| | | | TCTTCTCTGGATCTGGACCAAGGGCTTTGCAACTCCCAACTTGTT |
| | | | CTCGAGGAACCATCTTCTATCGTCCAGCTTGACTACAGTCAGAA |
| | | | GGTCTTGCTTGTGTCAACCCTTCAGCGAAGCCTTCTTTTTTATAC |
| | | | GGAAGAGAAGTCCGTTCGGCAGATTGGGACGCAACCCCGCAAA |
| | | | AGCACCGGTAAGTTTGGCGCATGTTTTATCCCAGGACTGTGTAA |
| | | | ACAATCTGACTTGACACTTTATGCCTCACGCCCAGGTCTGAGAC |
| | | | TCTGGAAAGCCGACGTACACGGCACTGTACAAGCTACATTTATC |
| | | | TTGAAGGACGCTTTCGCCGGCGGCGTTAAGCCCTTCGAACTGCA |
| | | | TCCCAGATTGGAGTCCCCCAATAGCGGTTCCTGTTCCCTTCCAG |
| | | | AGAGGCATCTGGGTCTTGTTTCCTGTTTCTTTCAGGAAGGCTGG |
| | | | GTGCTCAGCTGGAACGAATACTCAATATACCTGCTTGATACTGT |
| | | | GAACCAAGCTACTGTTGCGGGGCTGGAGGGAAGCGGTGATATT |
| | | | GTCAGCGTGTCATGCACTGAAAATGAGATTTTTTTTCTCAAAGG |
| | | | GGATCGAAATATAATTCGGATCTCATCCCGGCCCGAAGGACTTA |
| | | | CTAGCACTGTAAGAGACGGACTGGAGATGAGCGGATGTTCCGA |
| | | | ACGCGTGCACGTACAACAGGCTGAGAAGCTGCCCGGAGCTACT |
| | | | GTGAGTGAAACACGGTTGCGGGGCTCATCCATGGCTTCTTCAGT |
| | | | TGCAAGCGAACCGCGATCACGGTCATCATCATTGAACTCAACTG |
| | | | ATAGCGGCAGTGGACTGCTTCCCCCGGGATTGCAGGCTACCCCC |
| | | | GAACTCGGTAAAGGTTCTCAACCTCTCTCTCAGCGCTTCAACGC |
| | | | GATCTCCTCAGAAGATTTCGACCAGGAGTTGGTTGTTAAGCCCA |
| | | | TCAAAGTCAAGCGCAAAAAGAAAAAAAAAAAGACCGAAGGGG |
| | | | GCTCCCGGAGTACGTGCCACTCCTCTCTTGAGAGTACTCCGTGT |
| | | | AGTGAGTTTCCAGGCGACAGTCCGCAGTCTCTGAACACAGACCT |
| | | | TTTGAGTATGACAAGCTCAGTGCTTGGGAGCAGTGTAGACCAAC |
| | | | TTTCAGCTGAGTCACCGGATCAGGAGTCTTCTTTCAATGGCGAG |
| | | | GTTAATGGTGTACCACAGGAGAATACAGATCCCGAAACATTCA |
| | | | ACGTCCTCGAAGTCTCTGGTAGTATGCCAGATTCCCTCGCTGAA |
| | | | GAAGATGACATACGGACGGAAATGCCCCATTGCCACCACGCGC |
| | | | ATGGTCGAGAGCTTCTTAACGGAGCAAGGGAAGACGTAGGGGG |
| | | | CTCCGACGTAACGGGGTTGGGTGATGAACCGTGCCCGGCTGACG |
| | | | ACGGTCCCAATAGTACACAGTTGCCGTTCCAAGAACAGGACAGT |
| | | | TCCCCCGGCGCGCATGATGGAGAGGACATCCAGCCGATAGGTC |
| | | | CACAAAGTACTTTCTGCGAGGTTCCACTTCTCAACTCACTCACTG |
| | | | TCCCGAGCTCTCTTTCATGGGCCCCGTCAGCAGAACAATGGTTG |
| | | | CCAGGAACGCGCGCTGATGAAGGCTCCCCGGTTGAACCATCCCA |
| | | | AGAGCAGGACATACTGACATCCATGGAAGCTAGTGGGCACCTTT |
| | | | CTACTAATCTGTGGCACGCGGTCACGGATGACGATACGGGTCAG |
| | | | AAGGAGATACCAATCTCCGAGCGCGTACTCGGCTCCGTAGGCG |
| | | | GACAACTTACTCCCGTATCTGCGCTCGCAGCCAGTACCCACAAG |
| | | | CCATGGCTCGAGCAGCCACCTCGAGATCAAACTCTGACGTCATC |
| | | | AGATGAAGAGGACATTTACGCCCACGGGCTGCCCTCTTCTTCCT |
| | | | CAGAGACATCCGTTACAGAGTTGGGTCCGTCTTGCTCACAACAA |
| | | | GATTTGTCAAGACTTGGTGCCGAAGATGCTGGTCTCCTGAAACC |
| | | | TGATCAGTTTGCAGAGTCCTGGATGGGCTACTCTGGCCCGGGCT |
| | | | ATGGGATACTCTCCCTGGTCGTCTCCGAAAAGTATATTTGGTGT |
| | | | CTTGATTATAAGGGGGGTCTCTTCTGTTCTGCATTGCCGGGAGC |
| | | | AGGCTTGAGATGGCAAAAATTCGAGGATGCAGTACAACAAGTA |
| | | | GCCGTATCACCTAGTGGAGCATTGCTGTGGAAGATTGAGCAAAA |
| | | | ATCAAATCGGGCGTTTGCTTGTGGAAAGGTAACGATTAAAGGTA |
| | | | AGCGACACTGGTACGAAGCACTTCCCCAGGCCGTATTTGTTGCG |
| | | | CTGTCCGATGACACCGCCTGGATCATTCGAACATCTGGGGATTT |
| | | | GTATCTTCAAACGGGGCTCAGCGTAGATAGACCTTGTGCTCGGG |
| | | | CGGTAAAAGTGGACTGCCCATACCCCTTGTCACAGATTACTGCA |
| | | | CGAAACAACGTCGTTTGGGCTCTGACGGAGCAACGAGCTCTGCT |
| | | | CTATAGGGAAGGTGTAAGCTCTTTCTGCCCAGAAGGGGAACAGT |
| | | | GGAAGTGTGATATCGTATCAGAGCGCCAAGCTCTCGAACCCGTA |
| | | | TGTATCACACTGGGTGATCAGCAAACGCTTTGGGCTCTTGACAT |
| | | | ACATGGAAATCTTTGGTTTCGCACAGGCATCATTTCAAAAAAAC |
| | | | CACAGGGAGACGACGACCACTGGTGGCAAGTTTCTATCACGGA |
| | | | TTATGTCGTATTCGATCAGTGTTCACTTTTCCAGACCATAATCCA |
| | | | TGCCACGCATTCCGTGGCAACCGCGGCTCAAGCTCCCGTAGAAA |
| | | | AGGTGGCAGATAAACTCCGCATGGCATTTTGGTCACAACAGCTT |
| | | | CAGTGCCAACCTTCTTTGCTTGGTGTGAATAACTCTGGAGTTTGG |
| | | | ATTTCCAGTGGTAAAAACGAATTCCACGTTGCTAAGGGCAGCCT |
| | | | TATTGGTACTTATTGGAATCATGTTGTCCCTAGAGGAACAGCCT |
| | | | CTGCGACCAAGTGGGCCTTCGTACTTGCATCAGCGGCGCCAACT |
| | | | AAGGAGGGTTCATTCCTCTGGCTTTGCCAGTCTAGTAAAGACCT |
| | | | CTGTAGTGTGTCCGCACAAAGCGCCCAAAGCCGACCAAGCACC |
| | | | GTGCAGCTGCCGCCGGAGGCCGAGATGAGGGCGTACGCCGCAT |
| | | | GTCAAGACGCTTTGTGGGCGCTTGATTCTCTGGGTCAGGTATTT |
| | | | ATTAGGACTCTTTCAAAGAGCTGCCCCACGGGCATGCACTGGAC |
| | | | GCGGCTGGATCTCTCTCAGCTTGGGGCCGTAAAGCTCACTTCTC |
| | | | TTGCGTGTGGCAACCAGCATATCTGGGCATGCGATTCCAGAGGG |
| | | | GGAGTATACTTTCGAGTGGGCACACAACCTCTTAATCCGTCACT |
| | | | GATGCTTCCAGCGTGGATAATGATCGAGCCGCCGGTCCAACCCG |
| | | | CGGGTGTTTCACTTGTTAGTGTTCATTCATCTCCGAATGACCAAA |
| | | | TGCTCTGGGTCCTCGACTCTAGATGGAATGTTCACGTACGGACG |
| | | | GGTATTACAGAGGAGATGCCTGTAGGTACGGCTTGGGAGCATGT |
| | | | CCCTGGCCTGCAGGCATGTCAACTGGCTCTGTCCACTAGGACGG |
| | | | TCTGGGCCAGGTGTCCAAATGGGGACCTTGCCAGACGGTACGG |
| | | | AGTGACAGACAAGAATCCTGCAGGTGACTATTGGAAGAAAATA |
| | | | CCCGGTAGTGTAAGCTGTTTCACCGTCACGGCCTCAGATGAACT |
| | | | GTGGGCTGTGGGACCACCTGGTTACCTCCTCCAGCGCTTGACTA |
| | | | AGACATTCTCCCATTCCCACGGCACCCAGAAAAGTTCACAAGCT |
| | | | GCTATGCCACATCCAGAAGACCTGGAAGATGAGTGGGAAGTTA |
| | | | TTTGACCTTAACTTGTTTATTGCAGCTTATAATGGTTACAAATAA |
| | | | AGCAATAGCATCACAAATTTCACAAATAAAGCATTTTTTTCACT |
| | | | GCATTCTAGTTGTGGTTTGTCCAAACTCATCAATGTATCTTA |
| 6 | mU1a | 19 | ATGGAGGCGGTACTATGTAGATGAGAATTCAGGTGCAAACTGG |
| | Promoter | | GAAAAGCAACTGCTTCCAAATATTTGTGATTTTTACAGTGTAGT |
| | TECPR2 | | TTTGGAAAAACTCTTAGCCTACCAATTCTTCTAAGTGTTTTAAAA |
| | “STOP” | | TGTGGGAGCCAGTACACATGAAGTTATAGAGTGTTTTAATGAGG |
| | Proudfoot | | CTTAAATATTTACCGTAACTATGAAATGCTACGCATATCATGCT |
| | poly(A) | | GTTCAGGCTCCGTGGCCACGCAACTCATACTCCGCGGCCGCACT |
| | | | AGTGCCGCCATGGCGAGCATCTCAGAGCCGGTCACGTTTAGAGA |
| | | | GTTCTGTCCACTTTATTACCTTCTTAATGCAATTCCAACTAAAAT |
| | | | CCAGAAAGGATTTCGATCTATAGTAGTTTATTTGACCGCATTGG |
| | | | ATACGAACGGCGACTACATCGCGGTTGGCTCAAGCATCGGAAT |
| | | | GCTCTATTTGTACTGTCGGCACCTCAATCAGATGAGGAAATATA |
| | | | ACTTCGAAGGCAAGACCGAATCCATTACAGTIGTGAAGCTCCTC |
| | | | TCCTGTTTCGACGACCTGGTGGCCGCCGGCACCGCCTCCGGCCG |
| | | | AGTTGCAGTGTTCCAACTTGTATCAAGCCTCCCCGGCAGGAACA |
| | | | AGCAACTGAGAAGATTTGACGTTACGGGTATTCATAAGAATTCC |
| | | | ATAACGGCTCTGGCATGGAGCCCGAACGGGATGAAACTGTTCA |
| | | | GCGGTGACGATAAAGGGAAGATCGTGTACTCTTCTCTGGATCTG |
| | | | GACCAAGGGCTTTGCAACTCCCAACTTGTTCTCGAGGAACCATC |
| | | | TTCTATCGTCCAGCTTGACTACAGTCAGAAGGTCTTGCTTGTGTC |
| | | | AACCCTTCAGCGAAGCCTTCTTTTTTATACGGAAGAGAAGTCCG |
| | | | TTCGGCAGATTGGGACGCAACCCCGCAAAAGCACCGGTAAGTTT |
| | | | GGCGCATGTTTTATCCCAGGACTGTGTAAACAATCTGACTTGAC |
| | | | ACTTTATGCCTCACGCCCAGGTCTGAGACTCTGGAAAGCCGACG |
| | | | TACACGGCACTGTACAAGCTACATTTATCTTGAAGGACGCTTTC |
| | | | GCCGGCGGCGTTAAGCCCTTCGAACTGCATCCCAGATTGGAGTC |
| | | | CCCCAATAGCGGTTCCTGTTCCCTTCCAGAGAGGCATCTGGGTC |
| | | | TTGTTTCCTGTTTCTTTCAGGAAGGCTGGGTGCTCAGCTGGAACG |
| | | | AATACTCAATATACCTGCTTGATACTGTGAACCAAGCTACTGTT |
| | | | GCGGGGCTGGAGGGAAGCGGTGATATTGTCAGCGTGTCATGCA |
| | | | CTGAAAATGAGATTTTTTTTCTCAAAGGGGATCGAAATATAATT |
| | | | CGGATCTCATCCCGGCCCGAAGGACTTACTAGCACTGTAAGAGA |
| | | | CGGACTGGAGATGAGCGGATGTTCCGAACGCGTGCACGTACAA |
| | | | CAGGCTGAGAAGCTGCCCGGAGCTACTGTGAGTGAAACACGGT |
| | | | TGCGGGGCTCATCCATGGCTTCTTCAGTTGCAAGCGAACCGCGA |
| | | | TCACGGTCATCATCATTGAACTCAACTGATAGCGGCAGTGGACT |
| | | | GCTTCCCCCGGGATTGCAGGCTACCCCCGAACTCGGTAAAGGTT |
| | | | CTCAACCTCTCTCTCAGCGCTTCAACGCGATCTCCTCAGAAGATT |
| | | | TCGACCAGGAGTTGGTTGTTAAGCCCATCAAAGTCAAGCGCAAA |
| | | | AAGAAAAAAAAAAAGACCGAAGGGGGCTCCCGGAGTACGTGCC |
| | | | ACTCCTCTCTTGAGAGTACTCCGTGTAGTGAGTTTCCAGGCGAC |
| | | | AGTCCGCAGTCTCTGAACACAGACCTTTTGAGTATGACAAGCTC |
| | | | AGTGCTTGGGAGCAGTGTAGACCAACTTTCAGCTGAGTCACCGG |
| | | | ATCAGGAGTCTTCTTTCAATGGCGAGGTTAATGGTGTACCACAG |
| | | | GAGAATACAGATCCCGAAACATTCAACGTCCTCGAAGTCTCTGG |
| | | | TAGTATGCCAGATTCCCTCGCTGAAGAAGATGACATACGGACGG |
| | | | AAATGCCCCATTGCCACCACGCGCATGGTCGAGAGCTTCTTAAC |
| | | | GGAGCAAGGGAAGACGTAGGGGGCTCCGACGTAACGGGGTTGG |
| | | | GTGATGAACCGTGCCCGGCTGACGACGGTCCCAATAGTACACAg |
| | | | TTGCCGTTCCAAGAACAGGACAGTTCCCCCGGCGCGCATGATGG |
| | | | AGAGGACATCCAGCCGATAGGTCCACAAAGTACTTTCTGCGAG |
| | | | GTTCCACTTCTCAACTCACTCACTGTCCCGAGCTCTCTTTCATGG |
| | | | GCCCCGTCAGCAGAACAATGGTTGCCAGGAACGCGCGCTGATG |
| | | | AAGGCTCCCCGGTTGAACCATCCCAAGAGCAGGACATACTGAC |
| | | | ATCCATGGAAGCTAGTGGGCACCTTTCTACTAATCTGTGGCACG |
| | | | CGGTCACGGATGACGATACGGGTCAGAAGGAGATACCAATCTC |
| | | | CGAGCGCGTACTCGGCTCCGTAGGCGGACAACTTACTCCCGTAT |
| | | | CTGCGCTCGCAGCCAGTACCCACAAGCCATGGCTCGAGCAGCCA |
| | | | CCTCGAGATCAAACTCTGACGTCATCAGATGAAGAGGACATTTA |
| | | | CGCCCACGGGCTGCCCTCTTCTTCCTCAGAGACATCCGTTACAG |
| | | | AGTTGGGTCCGTCTTGCTCACAACAAGATTTGTCAAGACTTGGT |
| | | | GCCGAAGATGCTGGTCTCCTGAAACCTGATCAGTTTGCAGAGTC |
| | | | CTGGATGGGCTACTCTGGCCCGGGCTATGGGATACTCTCCCTGG |
| | | | TCGTCTCCGAAAAGTATATTTGGTGTCTTGATTATAAGGGGGGT |
| | | | CTCTTCTGTTCTGCATTGCCGGGAGCAGGCTTGAGATGGCAAAA |
| | | | ATTCGAGGATGCAGTACAACAAGTAGCCGTATCACCTAGTGGA |
| | | | GCATTGCTGTGGAAGATTGAGCAAAAATCAAATCGGGCGTTTGC |
| | | | TTGTGGAAAGGTAACGATTAAAGGTAAGCGACACTGGTACGAA |
| | | | GCACTTCCCCAGGCCGTATTTGTTGCGCTGTCCGATGACACCGC |
| | | | CTGGATCATTCGAACATCTGGGGATTTGTATCTTCAAACGGGGC |
| | | | TCAGCGTAGATAGACCTTGTGCTCGGGCGGTAAAAGTGGACTGC |
| | | | CCATACCCCTTGTCACAGATTACTGCACGAAACAACGTCGTTTG |
| | | | GGCTCTGACGGAGCAACGAGCTCTGCTCTATAGGGAAGGTGTA |
| | | | AGCTCTTTCTGCCCAGAAGGGGAACAGTGGAAGTGTGATATCGT |
| | | | ATCAGAGCGCCAAGCTCTCGAACCCGTATGTATCACACTGGGTG |
| | | | ATCAGCAAACGCTTTGGGCTCTTGACATACATGGAAATCTTTGG |
| | | | TTTCGCACAGGCATCATTTCAAAAAAACCACAGGGAGACGACG |
| | | | ACCACTGGTGGCAAGTTTCTATCACGGATTATGTCGTATTCGAT |
| | | | CAGTGTTCACTTTTCCAGACCATAATCCATGCCACGCATTCCGTG |
| | | | GCAACCGCGGCTCAAGCTCCCGTAGAAAAGGTGGCAGATAAAC |
| | | | TCCGCATGGCATTTTGGTCACAACAGCTTCAGTGCCAACCTTCTT |
| | | | TGCTTGGTGTGAATAACTCTGGAGTTTGGATTTCCAGTGGTAAA |
| | | | AACGAATTCCACGTTGCTAAGGGCAGCCTTATTGGTACTTATTG |
| | | | GAATCATGTTGTCCCTAGAGGAACAGCCTCTGCGACCAAGTGGG |
| | | | CCTTCGTACTTGCATCAGCGGCGCCAACTAAGGAGGGTTCATTC |
| | | | CTCTGGCTTTGCCAGTCTAGTAAAGACCTCTGTAGTGTGTCCGC |
| | | | ACAAAGCGCCCAAAGCCGACCAAGCACCGTGCAGCTGCCGCCG |
| | | | GAGGCCGAGATGAGGGCGTACGCCGCATGTCAAGACGCTTTGT |
| | | | GGGCGCTTGATTCTCTGGGTCAGGTATTTATTAGGACTCTTTCAA |
| | | | AGAGCTGCCCCACGGGCATGCACTGGACGCGGCTGGATCTCTCT |
| | | | CAGCTTGGGGCCGTAAAGCTCACTTCTCTTGCGTGTGGCAACCA |
| | | | GCATATCTGGGCATGCGATTCCAGAGGGGGAGTATACTTTCGAG |
| | | | TGGGCACACAACCTCTTAATCCGTCACTGATGCTTCCAGCGTGG |
| | | | ATAATGATCGAGCCGCCGGTCCAACCCGCGGGTGTTTCACTTGT |
| | | | TAGTGTTCATTCATCTCCGAATGACCAAATGCTCTGGGTCCTCG |
| | | | ACTCTAGATGGAATGTTCACGTACGGACGGGTATTACAGAGGA |
| | | | GATGCCTGTAGGTACGGCTTGGGAGCATGTCCCTGGCCTGCAGG |
| | | | CATGTCAACTGGCTCTGTCCACTAGGACGGTCTGGGCCAGGTGT |
| | | | CCAAATGGGGACCTTGCCAGACGGTACGGAGTGACAGACAAGA |
| | | | ATCCTGCAGGTGACTATTGGAAGAAAATACCCGGTAGTGTAAGC |
| | | | TGTTTCACCGTCACGGCCTCAGATGAACTGTGGGCTGTGGGACC |
| | | | ACCTGGTTACCTCCTCCAGCGCTTGACTAAGACATTCTCCCATTC |
| | | | CCACGGCACCCAGAAAAGTTCACAAGCTGCTATGCCACATCCAG |
| | | | AAGACCTGGAAGATGAGTGGGAAGTTATTTGATAATAGAGATC |
| | | | TGCTAGCTAATAAAAGATCCTTATTTTCATTGGATCTGTGTGTTG |
| | | | GTTTTTTGTGTG |

*The indicated expression cassette is encoded by the corresponding nucleic acid according to the sequence maps and sequence annotations in FIGs. 1-5 and 7.
**Additional sequence information may be found in corresponding FIGs. 1-5 and 7 and Tables 1-7.

TABLE 9
Nucleotide Sequence Information for Non-
limiting Examples of TECPR2 Construct
Promoters

| | Expression | SEQ | |
|---|---|---|---|
| | Construct | ID | Nucleotide Sequence |
| Construct* | Promoter | NO: | (5′→3′) |
| 1 | MECP2 | 8 | CAATTGAGGGCGTCACCGCTAAGGC |
| | | | TCCGCCCCAGCCTGGGCTCCACAAC |
| | | | CAATGAAGGGTAATCTCGACAAAGA |
| | | | GCAAGGGGTGGGGCGCGGGCGCGCA |
| | | | GGTGCAGCAGCACACAGGCTGGTCG |
| | | | GGAGGGCGGGGCGCGACGTCTGCCG |
| | | | TGCGGGGTCCCGGCATCGGTTGCGC |
| | | | GCGCGCTCCCTCCTCTCGGAGAGAG |
| | | | GGCTGTGGTAAAACCCGTCCGGAAA |
| 2 | JeT | 10 | GGGCGGAGTTAGGGCGGAGCCAATC |
| | | | AGCGTGCGCCGTTCCGAAAGTTGCC |
| | | | TTTTATGGCTGGGCGGAGAATGGGC |
| | | | GGTGAACGCCGATGATTATATAAGG |
| | | | ACGCGCCGGGTGTGGCACAGCTAGT |
| | | | TCCGTCGCAGCCGGGATTTGGGTCG |
| 3 | Human | 12 | CGGTTCTTGTTTGTGCTGCAGAGGG |
| | Synapsin | | CCCTGCGTATGAGTGCAAGTGGGTT |
| | (hSyn) | | TTAGGACCAGGATGAGGCGGGGTGG |
| | | | GGGTGCCTACCTGACGACCGACCCC |
| | | | GACCCACTGGACAAGCACCCAACCC |
| | | | CCATTCCCCAAATTGCGCATCCCCT |
| | | | ATCAGAGAGGGGGAGGGGAAACAGG |
| | | | ATGCGGCGAGGCGCGTGCGCACTGC |
| | | | CAGCTTCAGCACCGCGGACAGTGCC |
| | | | TTCGCCCCCGCCTGGCGGCGCGCGC |
| | | | CACCGCCGCCTCAGCACTGAAGGCG |
| | | | CGCTGACGTCACTCGCCGGTCCCCC |
| | | | GCAAACTCCCCTTCCCGGCCACCTT |
| | | | GGTCGCGTCCGCGCCGCCGCCGGCC |
| | | | CAGCCGGACCGCACCACGCGAGGCG |
| | | | CGAGATAGGGGGGCACGGGCGCGAC |
| | | | CATCTGCGCTGCGGCGCCGGC |
| 4 | Minimum | 14 | CAAAATCAACGGGACTTTCCAAAAT |
| | CMV | | GTCGTAATAACCCCGCCCCGTTGAC |
| | (minCMV) | | GCAAATGGGCGGTAGGCGTGTACGG |
| | | | TGGGAGGTCTATATAAGCAGAGCTC |
| | | | GTTTAGTGAACCGTCAGAATTCTCG |
| | | | AGTGATCGAAAGAGCCTGCTAAAGC |
| | | | AAAAAAGAA |
| 5 | minTK | 16 | TTCGCATATTAAGGTGACGCGTGTG |
| | | | GCCTCGAACACCGAGCGACCCTGCA |
| | | | GCGACCCGCTTAAAAGA |
| 6 | mU1a | 20 | ATGGAGGCGGTACTATGTAGATGAG |
| | | | AATTCAGGTGCAAACTGGGAAAAGC |
| | | | AACTGCTTCCAAATATTTGTGATTT |
| | | | TTACAGTGTAGTTTTGGAAAAACTC |
| | | | TTAGCCTACCAATTCTTCTAAGTGT |
| | | | TTTAAAATGTGGGAGCCAGTACACA |
| | | | TGAAGTTATAGAGTGTTTTAATGAG |
| | | | GCTTAAATATTTACCGTAACTATGA |
| | | | AATGCTACGCATATCATGCTGTTCA |
| | | | GGCTCCGTGGCCACGCAACTCATAC |
| | | | T |

*The indicated promoter is encoded by the corresponding nucleic acid according to the sequence maps and sequence annotations in FIGs. 1-5 and 7.

TABLE 10
Nucleotide Sequence Information for Non-limiting
Examples of TECPR2 Construct Components

| | | SEQ ID | |
|---|---|---|---|
| Construct | Component | NO: | Sequence |
| 1 | MECP2 | 8 | CAATTGAGGGCGTCACCGCTAAGGCTCCGCCCCAG |
| | promoter | | CCTGGGCTCCACAACCAATGAAGGGTAATCTCGAC |
| | | | AAAGAGCAAGGGGGGGGCGCGGGCGCGCAGGTG |
| | | | CAGCAGCACACAGGCTGGTCGGGAGGGCGGGGCG |
| | | | CGACGTCTGCCGTGCGGGGTCCCGGCATCGGTTGC |
| | | | GCGCGCGCTCCCTCCTCTCGGAGAGAGGGCTGTGG |
| | | | TAAAACCCGTCCGGAAA |
| 1 | Chimeric | 21 | GTAAGTATCAAGGTTACAAGACAGGTTTAAGGAGA |
| | intron | | CCAATAGAAACTGGGCTTGTCGAGACAGAGAAGAC |
| | | | TCTTGCGTTTCTGATAGGCACCTATTGGTCTTACTG |
| | | | ACATCCACTTTGCCTTTCTCTCCACAG |
| 1 | SV40 | 22 | AACTTGTTTATTGCAGCTTATAATGGTTACAAATAA |
| | poly(A) | | AGCAATAGCATCACAAATTTCACAAATAAAGCATT |
| | | | TTTTTCACTGCATTCTAGTTGTGGTTTGTCCAAACT |
| | | | CATCAATGTATCTTA |
| 2 | JeT | 10 | GGGCGGAGTTAGGGCGGAGCCAATCAGCGTGCGCC |
| | Promoter | | GTTCCGAAAGTTGCCTTTTATGGCTGGGCGGAGAA |
| | | | TGGGCGGTGAACGCCGATGATTATATAAGGACGCG |
| | | | CCGGGTGTGGCACAGCTAGTTCCGTCGCAGCCGGG |
| | | | ATTTGGGTCGCGGTTCTTGTTTGT |
| 2 | SV40 | 22 | AACTTGTTTATTGCAGCTTATAATGGTTACAAATAA |
| | Poly(A) | | AGCAATAGCATCACAAATTTCACAAATAAAGCATT |
| | | | TTTTTCACTGCATTCTAGTTGTGGTTTGTCCAAACT |
| | | | CATCAATGTATCTTA |
| 3 | hSyn | 12 | GCTGCAGAGGGCCCTGCGTATGAGTGCAAGTGGGT |
| | | | TTTAGGACCAGGATGAGGCGGGGTGGGGGTGCCTA |
| | | | CCTGACGACCGACCCCGACCCACTGGACAAGCACC |
| | | | CAACCCCCATTCCCCAAATTGCGCATCCCCTATCAG |
| | | | AGAGGGGGAGGGGAAACAGGATGCGGCGAGGCGC |
| | | | GTGCGCACTGCCAGCTTCAGCACCGCGGACAGTGC |
| | | | CTTCGCCCCCGCCTGGCGGCGCGCGCCACCGCCGC |
| | | | CTCAGCACTGAAGGCGCGCTGACGTCACTCGCCGG |
| | | | TCCCCCGCAAACTCCCCTTCCCGGCCACCTTGGTCG |
| | | | CGTCCGCGCCGCCGCCGGCCCAGCCGGACCGCACC |
| | | | ACGCGAGGCGCGAGATAGGGGGGCACGGGCGCGA |
| | | | CCATCTGCGCTGCGGCGCCGGC |
| 3 | MVM intron | 23 | AGAGGTAAGGGTTTAAGGGATGGTTGGTTGGTGGG |
| | | | GTATTAATGTTTAATTACCTGTTTTACAGGCCTGAA |
| | | | ATCACTTGGTTTTAGGTTGGG |
| 3 | MCS′ | 24 | AGCTTGATATCCCTGCAGGGC |
| 3 | SV40 | 22 | AACTTGTTTATTGCAGCTTATAATGGTTACAAATAA |
| | Poly(A) | | AGCAATAGCATCACAAATTTCACAAATAAAGCATT |
| | | | TTTTTCACTGCATTCTAGTTGTGGTTTGTCCAAACT |
| | | | CATCAATGTATCTTA |
| 4 | minCMV | 14 | CAAAATCAACGGGACTTTCCAAAATGTCGTAATAA |
| | | | CCCCGCCCCGTTGACGCAAATGGGCGGTAGGCGTG |
| | | | TACGGTGGGAGGTCTATATAAGCAGAGCTCGTTTA |
| | | | GTGAACCGTCAGAATTCTCGAGTGATCGAAAGAGC |
| | | | CTGCTAAAGCAAAAAAGAA |
| 4 | MVMi | 25 | AAGAGGTAAGGGTTTAAGGGATGGTTGGTTGGTGG |
| | | | GGTATTAATGTTTAATTACCTGTTTTACAGGCCTGA |
| | | | AATCACTTGGTTTTAGGTTGGG |
| 4 | SV40 | 22 | AACTTGTTTATTGCAGCTTATAATGGTTACAAATAA |
| | Poly(A) | | AGCAATAGCATCACAAATTTCACAAATAAAGCATT |
| | | | TTTTTCACTGCATTCTAGTTGTGGTTTGTCCAAACT |
| | | | CATCAATGTATCTTA |
| 5 | minTK | 16 | TTCGCATATTAAGGTGACGCGTGTGGCCTCGAACA |
| | | | CCGAGCGACCCTGCAGCGACCCGCTTAAAAGA |
| 5 | MVM intron | 23 | AGAGGTAAGGGTTTAAGGGATGGTTGGTTGGTGGG |
| | | | GTATTAATGTTTAATTACCTGTTTTACAGGCCTGAA |
| | | | ATCACTTGGTTTTAGGTTGGG |
| 5 | MCS′ | 24 | AGCTTGATATCCCTGCAGGGC |
| 5 | SV40 | 22 | AACTTGTTTATTGCAGCTTATAATGGTTACAAATAA |
| | poly(A) | | AGCAATAGCATCACAAATTTCACAAATAAAGCATT |
| | | | TTTTTCACTGCATTCTAGTTGTGGTTTGTCCAAACT |
| | | | CATCAATGTATCTTA |
| 6 | Mu1a | 20 | ATGGAGGCGGTACTATGTAGATGAGAATTCAGGTG |
| | promoter | | CAAACTGGGAAAAGCAACTGCTTCCAAATATTTGT |
| | | | GATTTTTACAGTGTAGTTTTGGAAAAACTCTTAGCC |
| | | | TACCAATTCTTCTAAGTGTTTTAAAATGTGGGAGCC |
| | | | AGTACACATGAAGTTATAGAGTGTTTTAATGAGGC |
| | | | TTAAATATTTACCGTAACTATGAAATGCTACGCATA |
| | | | TCATGCTGTTCAGGCTCCGTGGCCACGCAACTCATA |
| | | | CT |
| 6 | TECPR2 | 27 | ATGGCGAGCATCTCAGAGCCGGTCACGTTTAGAGA |
| | | | GTTCTGTCCACTTTATTACCTTCTTAATGCAATTCC |
| | | | AACTAAAATCCAGAAAGGATTTCGATCTATAGTAG |
| | | | TTTATTTGACCGCATTGGATACGAACGGCGACTAC |
| | | | ATCGCGGTTGGCTCAAGCATCGGAATGCTCTATTTG |
| | | | TACTGTCGGCACCTCAATCAGATGAGGAAATATAA |
| | | | CTTCGAAGGCAAGACCGAATCCATTACAGTIGTGA |
| | | | AGCTCCTCTCCTGTTTCGACGACCTGGTGGCCGCCG |
| | | | GCACCGCCTCCGGCCGAGTTGCAGTGTTCCAACTT |
| | | | GTATCAAGCCTCCCCGGCAGGAACAAGCAACTGAG |
| | | | AAGATTTGACGTTACGGGTATTCATAAGAATTCCA |
| | | | TAACGGCTCTGGCATGGAGCCCGAACGGGATGAAA |
| | | | CTGTTCAGCGGTGACGATAAAGGGAAGATCGTGTA |
| | | | CTCTTCTCTGGATCTGGACCAAGGGCTTTGCAACTC |
| | | | CCAACTTGTTCTCGAGGAACCATCTTCTATCGTCCA |
| | | | GCTTGACTACAGTCAGAAGGTCTTGCTTGTGTCAAC |
| | | | CCTTCAGCGAAGCCTTCTTTTTTATACGGAAGAGAA |
| | | | GTCCGTTCGGCAGATTGGGACGCAACCCCGCAAAA |
| | | | GCACCGGTAAGTTTGGCGCATGTTTTATCCCAGGA |
| | | | CTGTGTAAACAATCTGACTTGACACTTTATGCCTCA |
| | | | CGCCCAGGTCTGAGACTCTGGAAAGCCGACGTACA |
| | | | CGGCACTGTACAAGCTACATTTATCTTGAAGGACG |
| | | | CTTTCGCCGGCGGCGTTAAGCCCTTCGAACTGCATC |
| | | | CCAGATTGGAGTCCCCCAATAGCGGTTCCTGTTCCC |
| | | | TTCCAGAGAGGCATCTGGGTCTTGTTTCCTGTTTCT |
| | | | TTCAGGAAGGCTGGGTGCTCAGCTGGAACGAATAC |
| | | | TCAATATACCTGCTTGATACTGTGAACCAAGCTACT |
| | | | GTTGCGGGGCTGGAGGGAAGCGGTGATATTGTCAG |
| | | | CGTGTCATGCACTGAAAATGAGATTTTTTTTCTCAA |
| | | | AGGGGATCGAAATATAATTCGGATCTCATCCCGGC |
| | | | CCGAAGGACTTACTAGCACTGTAAGAGACGGACTG |
| | | | GAGATGAGCGGATGTTCCGAACGCGTGCACGTACA |
| | | | ACAGGCTGAGAAGCTGCCCGGAGCTACTGTGAGTG |
| | | | AAACACGGTTGCGGGGCTCATCCATGGCTTCTTCA |
| | | | GTTGCAAGCGAACCGCGATCACGGTCATCATCATT |
| | | | GAACTCAACTGATAGCGGCAGTGGACTGCTTCCCC |
| | | | CGGGATTGCAGGCTACCCCCGAACTCGGTAAAGGT |
| | | | TCTCAACCTCTCTCTCAGCGCTTCAACGCGATCTCC |
| | | | TCAGAAGATTTCGACCAGGAGTTGGTTGTTAAGCC |
| | | | CATCAAAGTCAAGCGCAAAAAGAAAAAAAAAAAG |
| | | | ACCGAAGGGGGCTCCCGGAGTACGTGCCACTCCTC |
| | | | TCTTGAGAGTACTCCGTGTAGTGAGTTTCCAGGCG |
| | | | ACAGTCCGCAGTCTCTGAACACAGACCTTTTGAGT |
| | | | ATGACAAGCTCAGTGCTTGGGAGCAGTGTAGACCA |
| | | | ACTTTCAGCTGAGTCACCGGATCAGGAGTCTTCTTT |
| | | | CAATGGCGAGGTTAATGGTGTACCACAGGAGAATA |
| | | | CAGATCCCGAAACATTCAACGTCCTCGAAGTCTCT |
| | | | GGTAGTATGCCAGATTCCCTCGCTGAAGAAGATGA |
| | | | CATACGGACGGAAATGCCCCATTGCCACCACGCGC |
| | | | ATGGTCGAGAGCTTCTTAACGGAGCAAGGGAAGAC |
| | | | GTAGGGGGCTCCGACGTAACGGGGTTGGGTGATGA |
| | | | ACCGTGCCCGGCTGACGACGGTCCCAATAGTACAC |
| | | | AgTTGCCGTTCCAAGAACAGGACAGTTCCCCCGGC |
| | | | GCGCATGATGGAGAGGACATCCAGCCGATAGGTCC |
| | | | ACAAAGTACTTTCTGCGAGGTTCCACTTCTCAACTC |
| | | | ACTCACTGTCCCGAGCTCTCTTTCATGGGCCCCGTC |
| | | | AGCAGAACAATGGTTGCCAGGAACGCGCGCTGATG |
| | | | AAGGCTCCCCGGTTGAACCATCCCAAGAGCAGGAC |
| | | | ATACTGACATCCATGGAAGCTAGTGGGCACCTTTC |
| | | | TACTAATCTGTGGCACGCGGTCACGGATGACGATA |
| | | | CGGGTCAGAAGGAGATACCAATCTCCGAGCGCGTA |
| | | | CTCGGCTCCGTAGGCGGACAACTTACTCCCGTATCT |
| | | | GCGCTCGCAGCCAGTACCCACAAGCCATGGCTCGA |
| | | | GCAGCCACCTCGAGATCAAACTCTGACGTCATCAG |
| | | | ATGAAGAGGACATTTACGCCCACGGGCTGCCCTCT |
| | | | TCTTCCTCAGAGACATCCGTTACAGAGTTGGGTCCG |
| | | | TCTTGCTCACAACAAGATTTGTCAAGACTTGGTGCC |
| | | | GAAGATGCTGGTCTCCTGAAACCTGATCAGTTTGC |
| | | | AGAGTCCTGGATGGGCTACTCTGGCCCGGGCTATG |
| | | | GGATACTCTCCCTGGTCGTCTCCGAAAAGTATATTT |
| | | | GGTGTCTTGATTATAAGGGGGGTCTCTTCTGTTCTG |
| | | | CATTGCCGGGAGCAGGCTTGAGATGGCAAAAATTC |
| | | | GAGGATGCAGTACAACAAGTAGCCGTATCACCTAG |
| | | | TGGAGCATTGCTGTGGAAGATTGAGCAAAAATCAA |
| | | | ATCGGGCGTTTGCTTGTGGAAAGGTAACGATTAAA |
| | | | GGTAAGCGACACTGGTACGAAGCACTTCCCCAGGC |
| | | | CGTATTTGTTGCGCTGTCCGATGACACCGCCTGGAT |
| | | | CATTCGAACATCTGGGGATTTGTATCTTCAAACGG |
| | | | GGCTCAGCGTAGATAGACCTTGTGCTCGGGCGGTA |
| | | | AAAGTGGACTGCCCATACCCCTTGTCACAGATTAC |
| | | | TGCACGAAACAACGTCGTTTGGGCTCTGACGGAGC |
| | | | AACGAGCTCTGCTCTATAGGGAAGGTGTAAGCTCT |
| | | | TTCTGCCCAGAAGGGGAACAGTGGAAGTGTGATAT |
| | | | CGTATCAGAGCGCCAAGCTCTCGAACCCGTATGTA |
| | | | TCACACTGGGTGATCAGCAAACGCTTTGGGCTCTT |
| | | | GACATACATGGAAATCTTTGGTTTCGCACAGGCAT |
| | | | CATTTCAAAAAAACCACAGGGAGACGACGACCACT |
| | | | GGTGGCAAGTTTCTATCACGGATTATGTCGTATTCG |
| | | | ATCAGTGTTCACTTTTCCAGACCATAATCCATGCCA |
| | | | CGCATTCCGTGGCAACCGCGGCTCAAGCTCCCGTA |
| | | | GAAAAGGTGGCAGATAAACTCCGCATGGCATTTTG |
| | | | GTCACAACAGCTTCAGTGCCAACCTTCTTTGCTTGG |
| | | | TGTGAATAACTCTGGAGTTTGGATTTCCAGTGGTAA |
| | | | AAACGAATTCCACGTTGCTAAGGGCAGCCTTATTG |
| | | | GTACTTATTGGAATCATGTTGTCCCTAGAGGAACA |
| | | | GCCTCTGCGACCAAGTGGGCCTTCGTACTTGCATCA |
| | | | GCGGCGCCAACTAAGGAGGGTTCATTCCTCTGGCT |
| | | | TTGCCAGTCTAGTAAAGACCTCTGTAGTGTGTCCGC |
| | | | ACAAAGCGCCCAAAGCCGACCAAGCACCGTGCAG |
| | | | CTGCCGCCGGAGGCCGAGATGAGGGCGTACGCCGC |
| | | | ATGTCAAGACGCTTTGTGGGCGCTTGATTCTCTGGG |
| | | | TCAGGTATTTATTAGGACTCTTTCAAAGAGCTGCCC |
| | | | CACGGGCATGCACTGGACGCGGCTGGATCTCTCTC |
| | | | AGCTTGGGGCCGTAAAGCTCACTTCTCTTGCGTGTG |
| | | | GCAACCAGCATATCTGGGCATGCGATTCCAGAGGG |
| | | | GGAGTATACTTTCGAGTGGGCACACAACCTCTTAA |
| | | | TCCGTCACTGATGCTTCCAGCGTGGATAATGATCG |
| | | | AGCCGCCGGTCCAACCCGCGGGTGTTTCACTTGTTA |
| | | | GTGTTCATTCATCTCCGAATGACCAAATGCTCTGGG |
| | | | TCCTCGACTCTAGATGGAATGTTCACGTACGGACG |
| | | | GGTATTACAGAGGAGATGCCTGTAGGTACGGCTTG |
| | | | GGAGCATGTCCCTGGCCTGCAGGCATGTCAACTGG |
| | | | CTCTGTCCACTAGGACGGTCTGGGCCAGGTGTCCA |
| | | | AATGGGGACCTTGCCAGACGGTACGGAGTGACAGA |
| | | | CAAGAATCCTGCAGGTGACTATTGGAAGAAAATAC |
| | | | CCGGTAGTGTAAGCTGTTTCACCGTCACGGCCTCA |
| | | | GATGAACTGTGGGCTGTGGGACCACCTGGTTACCT |
| | | | CCTCCAGCGCTTGACTAAGACATTCTCCCATTCCCA |
| | | | CGGCACCCAGAAAAGTTCACAAGCTGCTATGCCAC |
| | | | ATCCAGAAGACCTGGAAGATGAGTGGGAAGTTATT |
| 6 | Proudfoot | 26 | TAATAAAAGATCCTTATTTTCATTGGATCTGTGTGT |
| | poly(A) | | TGGTTTTTTGTGTG |

### Example 2. In Vivo Expression Studies

The five rAAVs comprising the TECPR2 constructs are made as described above and administered via the facial vein to newborn C57BL/6 mice (n=6-10/group) at 5×10¹³ vector genomes per kg subject (vg/kg). Two to four weeks after rAAV dosing, nervous system tissues from mice subjects are harvested and whole cell lysates are analyzed for TECPR2 expression using ELISA and/or immunoblot and/or qPCR.

Separately, the five rAAVs comprising the TECPR2 constructs are made as described above and administered via injection into the jugular vein, injection into the peritoneum, or intracerebroventricular injection of 5-7 weeks old C57BL/6 mice (n=6-10/group) at three different doses: 1×10¹³ vg/kg, 5×10¹³ vg/kg, and 1×10¹⁴ vg/kg. One month after rAAV dosing, nervous system tissues are harvested and whole cell lysates are analyzed for TECPR2 expression using ELISA and/or immunoblot and/or qPCR.

### Example 3. Restoration of TECPR2 Expression in Mammals In Vivo

In this experiment, genetic mouse models of TECPR2-associated diseases are utilized to study the efficacy of the TECPR2 constructs provided herein. The models include heterozygous null animals (TECPR2 (+/−)) or MMRC strain #042112-JAX transgenic mice.

rAAV particles comprising the TECPR2 constructs are made as described above and delivered via single IV injection to presymptomatic and/or symptomatic TECPR2 mouse models using different doses. Non-limiting examples of doses include 1×10¹³ vg/kg, 5×10¹³ vg/kg, and 1×10¹⁴ vg/kg. Nervous system tissue samples are collected and whole tissue lysates are analyzed for AAV biodistribution by qPCR and for human TECPR2 expression by immunoblot and/or ELISA. In addition, tissues are monitored for histopathology. Therapeutic effects of the rAAV described herein are assessed.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A nucleic acid comprising a TECPR2 sequence flanked on each side by an inverted terminal repeat sequence.

2. The nucleic acid of claim 1, wherein the TECPR2 sequence is a human TECPR2 coding sequence.

3. The nucleic acid of claim 1 or 2, wherein the TECPR2 sequence is operably linked to one or more regulatory elements.

4. A nucleic acid comprising a human TECPR2 sequence operably linked to one or more regulatory elements, wherein the nucleic acid is a recombinant nucleic acid.

5. The nucleic acid of claim 3 or 4, wherein the one or more regulatory elements comprise a promoter.

6. The nucleic acid of any one of claims 3-5, wherein the one or more regulatory elements comprise an enhancer.

7. The nucleic acid of any one of claims 1-6, wherein the human TECPR2 sequence is codon-optimized for expression in human cells.

8. The nucleic acid of any one of claims 1-7, wherein the human TECPR2 sequence comprises a nucleic acid sequence having at least about 85% sequence identity to sequence of SEQ ID NO: 17 or 27.

9. The nucleic acid of any one of claims 1-8, wherein the human TECPR2 sequence comprises a nucleic acid sequence that comprises the sequence of SEQ ID NO: 17 or 27.

10. The nucleic acid of any one of claims 5-9, wherein the promoter comprises a neuron-specific promoter.

11. The nucleic acid of any one of claims 5-10, wherein the promoter is selected from the group consisting of: TECPR2, mouse MECP2, JeT, human synapsin, minCMV, minTK, mU1A, and combinations thereof.

12. The nucleic acid of any one of any one of claims 5-11, wherein the promoter comprises a nucleic acid sequence having at least about 85% sequence identity to the sequence of SEQ ID NO: 8, 10, 12, 14, 16, or 20.

13. The nucleic acid of any one of claims 5-12, wherein the promoter comprises a nucleic acid sequence comprising the sequence of SEQ ID NO: 8, 10, 12, 14, 16, 20.

14. The nucleic acid of any one of claims 1-13, wherein the nucleic acid has at least about 85% sequence identity to the sequence of SEQ ID NOs: 7, 9, 11, 13, 15, 17, 19, or 27.

15. The nucleic acid of any one of claims 1-13, wherein the nucleic acid comprises the sequence of SEQ ID NO: 7, 9, 11, 13, 15, 17, 19, or 27.

16. The nucleic acid of any one of claims 1-15, wherein the nucleic acid comprises an MVMi intron.

17. The nucleic acid of any one of claims 1-16, wherein the nucleic acid comprises a minimal polyA signal.

18. A nucleic acid comprising an expression cassette comprising a human TECPR2 sequence, wherein the TECPR2 sequence is operably linked to a promoter and optionally an enhancer element, and wherein the expression cassette is flanked on each side by an inverted terminal repeat sequence.

19. The nucleic acid of claim 18, wherein the human TECPR2 sequence is codon-optimized for expression in human cells.

20. The nucleic acid of claim 18 or 19, wherein the human TECPR2 sequence comprises a nucleic acid sequence having at least about 85% sequence identity to sequence of SEQ ID NO: 17 or 27.

21. The nucleic acid of any one of 18-20, wherein the human TECPR2 sequence comprises the nucleic acid sequence of SEQ ID NO: 17 or 27.

22. The nucleic acid of any one of claims 18-21, wherein the promoter comprises a neuron-specific promoter.

23. The nucleic acid of any one of claims 18-22, wherein the promoter is selected from the group consisting of: TECPR2, mouse MECP2, JeT, human synapsin, minCMV, minTK, mU1A, and combinations thereof.

24. The nucleic acid of any one of claims 18-23, wherein the promoter comprises a nucleic acid sequence having at least about 85% sequence identity to the sequence of SEQ ID NO: 8, 10, 12, 14, 16, or 20.

25. The nucleic acid of any one of claims 18-24, wherein the promoter comprises a nucleic acid sequence comprising the sequence of SEQ ID NO: 8, 10, 12, 14, 16, or 20.

26. The nucleic acid of any one of claims 18-25, wherein the expression cassette has at least about 85% sequence identity to the sequence of SEQ ID NOs: 7, 9, 11, 13, 15, 17, 19, or 27.

27. The nucleic acid of any one of claims 18-26, wherein the expression cassette comprises the sequence of SEQ ID NO: 7, 9, 11, 13, 15, 17, 19, or 27.

28. The nucleic acid of any one of claims 18-27, wherein the expression cassette comprises an MVMi intron.

29. The nucleic acid of any one of claims 18-28, wherein the expression cassette comprises a minimal polyA signal.

30. The nucleic acid of any one of claims 1-29, wherein the nucleic acid is provided in a recombinant adeno-associated virus (rAAV) vector.

31. The nucleic acid of claim 30, wherein the nucleic acid is a single-stranded or self-complementary rAAV nucleic acid vector.

32. The nucleic acid of claim 30 or 31, wherein the rAAV vector comprises a nucleic acid sequence that is at least about 85% identical to one or more coding sequences and/or sequence elements of any one of SEQ ID NOs: 1-5, 7, 9, 11, 13, 15, 17, 18, 19, or 27.

33. The nucleic acid of claim 31 or 32, wherein the rAAV vector comprises a nucleic acid sequence as set forth in one or more coding sequences and/or sequence elements of any one of SEQ ID NOs: 1-5, 7, 9, 11, 13, 15, 17, 18, 19, or 27.

34. A recombinant adeno-associated virus (rAAV) particle comprising a recombinant genome comprising the nucleic acid of any one of claims 1-33.

35. The rAAV particle of claim 34, wherein the rAAV particle is an AAV9 particle.

36. A composition comprising at least one or a plurality of the rAAV particle of claim 34 or 35.

37. The composition of claim 36, further comprising a pharmaceutically acceptable carrier.

38. A method of treating a TECPR2-associated disease, the method comprising: administering a therapeutically effective amount of rAAV vector comprising:
(a) a nucleic acid comprising a human TECPR2 sequence, wherein the TECPR2 sequence is operably linked to a promoter and optionally an enhancer element; and
(b) inverted terminal repeat sequences which flank the nucleic acid of (a),
wherein said administration results in expression of a therapeutically effective amount of human TECPR2, thereby treating the TECPR2-associated disease or disorder.

39. The method of claim 38, wherein the rAAV is administered via an intravenous injection.

40. The method of claim 38, wherein the rAAV is administered via an injection into cerebrospinal fluid.

41. The method of any one of claims 38-40, wherein between about 1×10¹³ and about 1×10¹⁴ rAAV vector genomes are administered.

42. The method of any one of claims 38-41, wherein the rAAV vector or the nucleic acid comprises a sequence that is at least about 85% identical to one or more coding sequences and/or sequence elements of any one of SEQ ID NOs: 1-5 or 7-27, optionally wherein the rAAV vector or the nucleic acid comprises 85% identity to a sequence in any one of SEQ ID NOs: 1-5, 7, 9, 11, 13, 15, 17, 18, 19, or 27.

43. The method of any one of claims 38-41, wherein the nucleic acid comprises one or more coding sequences and/or sequence elements of any one of SEQ ID NOs: 1-5 or 7-27, optionally wherein the rAAV vector or the nucleic acid comprises one or more coding sequences and/or sequence elements of any one of SEQ ID NOs: 1-5, 7, 9, 11, 13, 15, 17, 18, 19, or 27.

44. A method of increasing expression of human TECPR2 in a target cell, the method comprising: contacting a target cell with a plurality of rAAV particles comprising a nucleic acid comprising a cassette comprising a functional human TECPR2 sequence operably linked to a promoter and optionally an enhancer element, wherein the cassette is flanked on each side by an inverted terminal repeat sequence.

45. The method of claim 44, wherein the step of contacting results in increased expression of functional human TECPR2 in the target cell as compared to prior to the contacting, thereby increasing the expression of functional human TECPR2.

46. The method of claim 44 or 45, wherein the contacting is performed in vivo.

47. The method of any one of claims 44-46, wherein the method is used for the treatment of a TECPR2-associated disease or disorder.

48. Use of the nucleic acid of any one of claims 1-33, the rAAV particle of claim 34 or 35, or the composition of claim 36 or 37, in the manufacture of a medicament for the treatment of a TECPR2-associated disease or disorder.

49. Use of the nucleic acid of any one of claims 1-33, the rAAV particle of claim 34 or 35, or the composition of claim 36 or 37, for the treatment of a TECPR2-associated disease or disorder.

50. A cell comprising the nucleic acid of any one of claims 1-33.

51. The cell of claim 50, wherein the cell is a mammalian cell.

52. The cell of claim 51, wherein the cell is a human cell.

53. The cell of any one of claims 50-52, wherein the cell is a cell of the nervous system.

54. The cell of any one of claims 50-53, wherein the cell comprises a TECPR2 mutation.

55. The cell of claim 50, wherein the cell is a HEK293 cell or a C2C12 myoblast cell.

56. A cell population comprising one or a plurality of the cell of any one of claims 50-56.
